(19) United States
(12) Patent Application Publication
(10) Pub. No.: US 2026/0166521 A1
(43) Pub. Date: Jun. 18, 2026

(54) Affinity Matrix Compositions and Methods of Use Thereof

(71) Applicant: GEORGE MASON UNIVERSITY, Fairfax, VA (US)

(72) Inventors: Rocio Solange Prisby, Vienna, VA (US); Alessandra Luchini Kunkel, Burke, VA (US); Lance Allen Liotta, Bethesda, MD (US); Cade Jameson Skislak, Centreville, VA (US)

(21) Appl. No.: 19/355,519

(22) Filed: Oct. 10, 2025

Related U.S. Application Data
(60) Provisional application No. 63/706,477, filed on Oct. 11, 2024.

(51) Int. Cl. B01J 20/26; B01D 15/38; B01J 20/32; C12Q 1/689; G01N 33/569; G01N 33/68

(52) U.S. Cl. CPC B01J 20/267; B01D 15/3804; B01J 20/3208; B01J 20/3219; B01J 20/3282; C12Q 1/689; G01N 33/5695; G01N 33/6848; G01N 2333/20; G01N 2333/35

(57) ABSTRACT

The present disclosure relates to affinity matrix compositions, kits comprising the same, and methods of using same for separating analytes, including biomarkers, from liquid samples. In certain embodiments, the methods described herein are useful for diagnosing and/or treating, preventing, or ameliorating a disease or disorder in a subject, including but not limited to infections with pathogens (e.g., borreliosis and tuberculosis).

### CROSS-REFERENCE TO RELATED APPLICATION

This application claims priority under 35 U.S.C. § 119(e) to U.S. Provisional Patent Application No. 63/706,477, filed Oct. 11, 2024, which is incorporated herein by reference in its entirety.

### REFERENCE TO AN ELECTRONIC SEQUENCE LISTING

The XML file named “387351-7004US1 —Sequence Listing.xml” created on Oct. 8, 2025, comprising 8,105 bytes, is incorporated herein by reference in its entirety.

### BACKGROUND

The detection and analysis of biomarkers in biofluids are crucial for diagnosing and understanding a wide array of infectious diseases, including Lyme disease, tuberculosis, and other pathogen-induced conditions. Traditional diagnostic methods, often reliant on serological approaches, can fall short in sensitivity and specificity, particularly when dealing with diseases that exhibit complex pathogen-host interactions and subtle biochemical signatures. Moreover, biofluids like urine provide a non-invasive means to explore these interactions, yet the low concentration and susceptibility to degradation of peptides and proteins within these fluids present significant obstacles.

Urine, with its potential to reflect the physiological state of a host, offers a promising pathway for monitoring infections and understanding the underlying mechanisms of diseases. However, existing technologies struggle to efficiently capture and preserve the biomarkers necessary for comprehensive analysis. This limitation hampers the ability to diagnose diseases early, track their progression, and develop targeted treatment strategies, especially for conditions characterized by neuroinflammation or other systemic effects.

Thus, there is a need in the art for an advanced composition and process capable of efficiently capturing and analyzing small molecules and peptides from biofluids, such as urine, to enhance the detection and understanding of various infectious diseases. The disclosure addresses this unmet need.

### SUMMARY OF THE INVENTION

In one aspect, the disclosure provides an affinity matrix comprising a biocompatible fibrous material, a hydrogel, and an affinity label, wherein the hydrogel is disposed on the biocompatible fibrous material, and wherein the affinity label is associated with at least one of the biocompatible fibrous material and the hydrogel.

In another aspect, the disclosure provides a method for preparing the affinity matrix of the disclosure, the method comprising: contacting a fibrous material sheet with an affinity label to provide an affinity labeled fibrous material; placing the affinity labeled fibrous material in a sealable vessel; and contacting the affinity labeled fibrous material with a monomer solution comprising a vinyl monomer, a divinyl crosslinker, and a radical initiator under an anaerobic condition.

In another aspect, the disclosure provides a method for separating an analyte from a sample, the method comprising: contacting a liquid sample with the affinity matrix of the disclosure to provide an analyte-adsorbed affinity matrix and a supernatant; separating the analyte-adsorbed affinity matrix and the supernatant; and contacting the analyte-adsorbed affinity matrix with an eluting solution to provide an analyte solution.

In another aspect, the disclosure provides a method for diagnosing a disease or disorder in a subject, the method comprising: contacting a liquid biological sample obtained from the subject with the affinity matrix of the disclosure to provide an analyte-adsorbed affinity matrix and a supernatant; separating the analyte-adsorbed affinity matrix and the supernatant; contacting the analyte-adsorbed affinity matrix with an eluting solution to provide an analyte solution; and detecting a biomarker associated with the disease or disorder in the analyte solution.

In another aspect, the disclosure provides a method for treating a disease or disorder in a subject, the method comprising: contacting a liquid biological sample obtained from the subject with the affinity matrix of the disclosure to provide an analyte-adsorbed affinity matrix and a supernatant; separating the analyte-adsorbed affinity matrix and the supernatant; contacting the analyte-adsorbed affinity matrix with an eluting solution to provide an analyte solution; detecting a biomarker associated with the disease or disorder in the analyte solution; and administering to the subject a therapeutic agent effective to treat the disease or disorder.

In another aspect, the disclosure provides a kit comprising the affinity matrix of the disclosure, a liquid collection device, and instructions for preparing and storing an analyte-adsorbed affinity matrix in the collection device.

### BRIEF DESCRIPTION OF THE DRAWINGS

The drawings illustrate generally, by way of example, but not by way of limitation, various embodiments of the present application.

FIG. 1: Transmission of Lyme disease. B. burgdorferi is transmitted through a tick bite; erythema migrants, a red rash, and a typical bull's eye can be observed on the attachment site.

FIGS. 2A-2F: Innovative solid-based affinity enrichment technology. FIG. 2A: Outline of the experimental procedure to capture Borrelia-derived biomarkers and EVs from patient urine.

FIGS. 2B-2D: Western Blot showing the presence of OspA (FIG. 2B), Fagellin (FIG. 2C), and OspC (FIG. 2D), respectively, in patient urine samples. FIGS. 2E-2F: SDS PAGE analysis shows a successful protein capture and elution using PDM baited with Sudan IV (FIG. 2E) and Sudan Black B (FIG. 2F) with urine samples.

FIGS. 3A-3D: Solid-based affinity binding enrichment technologies. FIG. 3A: Atomic force microscopy images of core-shell nanoparticles NIPA/AAc and NIPAm/C.B. particles. FIG. 3B: Hydrogel NIPA-based nanoparticles used to concentrate Toxoplasmosis urine-derived antigens to increase detectability in mass spectrometry analysis. FIG. 3C: Diagram of glass wool fibers used to capture Tuberculosis urine-derived DNA for posterior analysis. FIG. 3D: Urigami cup was designed as a collection device containing glass wool affinity fibers. Patients urinate into the collection container, incubate, and remove the urine. Target analytes are sent for proteomics analysis.

FIGS. 4A-4C: Nylon-based affinity binding enrichment technologies. FIG. 4A: Nylon-based affinity net dyed with Sudan Black B. FIG. 4B: Diagram showing molecular interactions between the affinity net and the hydrophobic portion of the Sudan Black B and the anionic terminal of nylon 6 with the ionic portion of Sudan Black B. FIG. 4C: Outline of the experimental procedure to capture Borrelia-derived biomarkers and EVs from patient urine and posterior analysis via mass spectrometry.

FIGS. 5A-5C: Innovative solid-based affinity enrichment technology. FIG. 5A: FT-IR spectrum of NIPA-BIS-AA hydrogel polymerized onto nylon 6. Spectrum was collected using Cary 630 FTIR—Agilent Technology with the following setup, range: 4000-650, sample scans: 32, sampling module: transmission. Notice that C═O is associated since the stretching is at 1636 cm⁻¹, while free stretching can be found at 1690 cm⁻¹. FIG. 5B: 22% NIPA-BIS-AA hydrogel polymerized onto nylon 6 using BIS as a crosslinker, FIG. 5C: dyed with parosaniline base dye.

FIGS. 6A-6E: Isolation of bacterial MVs and inflammation response by human neuronal cells upon exposure to bacterial-related antigens. FIG. 6A: Electron microscope images of isolated MVs from Lactobacillus rhamnosus. FIG. 6B: Transmission electron micrographs of Borrelia-derived MVs showing spherical shape and single bilayer membrane. FIG. 6C: Borrelia burgdorferi electron microscope images with embedded MVs. Bars: B: 100 nm, C: 500 nm. FIG. 6D: Differentially expressed genes after 48 hrs exposure of Borrelia burgdorferi to human choroid plexus epithelial cells measured by RT-qPCR. FIG. 6E: Concentration of selected cytokines in supernatant from human choroid plexus epithelial infected cells after 48 hrs. exposure to Borrelia burgdorferi.

FIG. 7: Development of DGP and PDM. On the left, grafting of nylon 6 sheet via free-radical polymerization with acrylamide-based hydrogel. On the right, the incorporation of dye molecules through adsorption or chemical reaction.

FIG. 8: provides a scheme showing certain aims of the disclosure.

FIG. 9: Image showing certain innovative aspects of the disclosure.

FIG. 10: Workflow showing DGP (left) and PDM (right) synthesis and preparation. Table on the right shows all the dyes successfully incorporated onto nylon through systematic experiments using a water-based bath.

FIG. 11: Diagram showing incorporation of different dyes with different molecular structures onto nylon 6 fibers.

FIGS. 12A-12B: FIG. 12A: The general reaction between n-isopropylacrylamide (NIPA) and N, N′-Methylenebisacrylamide (BIS) using the APS and TEMED initiator system. FIG. 12B: Reaction between APS and TEMED initiator system.

FIG. 13: Developmental Progression from Initial Experiments to Final Prototype. The iterative process of designing the ultimate prototype begins with foundational experiments and leads to two prototype versions before reaching the final design.

FIGS. 14A-14C: Depiction of the DyeGel Polyamide mold prototype. FIG. 14A: Presents a top-down view, capturing the spatial arrangement between the top and bottom plates. FIG. 14B: Gives a side profile, elucidating the structural hierarchy. Scale bar shows a thickness of 1 mm. FIG. 14C: Offers an angled side perspective, underscoring the distinction between the top glass, silicone layer, parafilm layer, and the bottom glass. The layering is optimized to ensure effective polymerization, minimal substrate dispersion, and protection from external contaminants.

FIGS. 15A-15C: DyeGel Polyamide (DGP) development. FIG. 15A: Showcases a polyamide sample with Bismarck Brown dye within a glass mold, below, nylon—6 dyed with Acid Black 48 and Cibacron Red. FIG. 15B: Optical microscope image showing the intertwined blue filaments (Cibacron Blue) with acrylamide-based hydrogel (200X). Adjacently, the molecular configurations of PNIPAM-co-AA and Nylon 6. FIG. 15C: DGP dye free and dyed with pararosaniline base dye.

FIGS. 16A-16D: DyeGel Polyamide (DGP) development processes. FIG. 16A: NIPA-BIS molecular structure and its respective sample. FIG. 16B: ACM-BIS molecular structure and samples dyed with Cibacron blue and Bismarck Brown. FIG. 16C: Portrays the NIPA-BIS sample with % T 15% and 20% and its molecular structure. FIG. 16D: Presents the NIPA-BIS-VSA sample and chemical structure, highlighting the incorporation of VSA.

FIGS. 17A-17D: Characterization of DGP and PDG. FIG. 17A: FT-IR of air-dried DGP (NIPA-BIS-AA). FIG. 17B: (top) Spectral scanning of nylon, Bismarck Brown, and nylon-Bismarck Brown, notice the bathochromic shift; (bottom) spectral scanning of nylon, Remazol Brilliant Blue, nylon—RBBr showing absence of bathochromic shifting. FIG. 17C: Calibration curve for acid black 48 in 22% CaCl₂/MeOH. FIG. 17D: Nylon dyed with acid black 48 with a table showing homogenous incorporation of dye.

FIGS. 18A-18B: Potential pitfalls. FIG. 18A: FT-IR of different thicknesses of AH-70 polymer films; intensity increases as thickness decreases. FIG. 18B: FT-IR of nylon sample before and after methylene blue is incorporated. Change in intensity is highlighted.

FIGS. 19A-19B: Potential pitfalls. FIG. 19A: Reactive Red 120 dye in 22% CaCl₂/MeOH—partially soluble FIG. 19B: Solubility blot of Acid Black 48 (top) and Safranin O (bottom).

FIG. 20: Schematic representation of the investigative process for the sequestration capabilities of PDM and DGP. The workflow commences with patient urine samples, undergoes sequential stages of incubation, elusion, and analysis, and culminates in biomarker identification through techniques like LC-MS analysis and Western blot.

FIG. 21: Diagram showing how proteins in the urine bind to PDG and DGP.

FIG. 22: A three-step overview detailing the assessment of the dynamic range of biomarkers captured by PDM/DGP. The process begins with urine-derived proteins undergoing incubation with PDM or DGP (Step 1), followed by quantification techniques such as Western blot and mass spectrometry (Step 2), and concludes with an evaluation of the analytical performance showcasing the dynamic range of detected protein concentrations (Step 3).

FIG. 23: This illustration provides an insight into a research plan focused on selected patient samples, specifically evaluating Lyme disease serology. The left side of the figure lists various patients by their IDs, highlighting their Lyme serology results, gender, and treatment status. All patients are observed to have positive serology but have not undergone any treatment. On the right, bar graphs depict the distribution of symptoms between male and female patients, showing common symptoms like joint pain, fatigue, fever, neurological disorders, and other unspecified issues. Two histograms at the bottom present the age distribution for both male and female patients in various age groups.

FIGS. 24A-24G: FIG. 24A: Nylon 6 bound to fluorescently labeled chicken lysozyme. FIG. 24B: Nylon grafted with 22% ACM-Bis bound to fluorescently labeled lysozyme (center of the gel). FIG. 24 C: Nylon grafted with 22% ACM-Bis bound to fluorescently labeled lysozyme (edge of the gel). FIG. 24D: Nylon 6 bound to fluorescently labeled chicken lysozyme. FIG. 24E: Nylon grafted with 20% NIPA-Bis functionalized with allylamine bound to fluorescently labeled lysozyme (center of the gel). FIG. 24F: Nylon grafted with 20% NIPA-Bis functionalized with allylamine bound to fluorescently labeled lysozyme (edge of the gel). FIG. 24G: Chicken lysozyme was labeled using Alexa 660, degree of labeling of chicken lysozyme was measured to ensure optimal labeling with a value of 1.

FIGS. 25A-25C: The image presents three panels. FIGS. 25A-25B: showcasing protein/biomarker panel 1 (FIG. 25A) and panel 2 (FIG. 25B) enrichment via gel electrophoresis bands when urine is incubated with PDM. The highlighted red boxes across all panels accentuate regions with significant protein enhancement when compared to control samples. FIG. 25C: Western blot on the right indicates the presence of flagellin antibodies in patients suspected of having Lyme disease versus control samples.

FIGS. 26A-26B: The image showcases a proteomics analysis of urine-derived biomarkers. FIG. 26A: gel electrophoresis panel display protein bands from various patent samples (1). FIG. 26B: gel electrophoresis panel display protein bands from various patent samples (2).

FIG. 27: The illustration depicts the process of studying the effects of Borrelia-derived microvesicles (MV) on neuronal cells. Starting with incubation of Borrelia culture, microvesicles are harvested and subjected to analysis. These MVs are introduced to neuronal cell cultures. The cellular response is then evaluated through methods such as ELISA to assess the release of inflammatory mediators and RT-PCR for gene expression changes.

FIG. 28: The diagram outlines the methods for isolating and characterizing Microvesicles (MVs). Key steps involve proteomic and genomic analyses, size determination, and lipid profiling using Thin Layer Chromatography (TLC).

FIG. 29: The image illustrates the inflammatory response of HMC3 microglial cells to Borrelia-derived microvesicles (MVs). Techniques like RT-PCR, Western Blot, and ELISA are employed to study cytokine expression and activation pathways.

FIGS. 30A-30F: FIG. 30A: observed exosomes derived from Lactobacillus sp. that were extracted in-house. FIG. 30B: displays Borrelia cultured in-house and stained with FITC and DAPI. FIGS. 30C-30D: present Borrelia cultures obtained from an external group with 100 nm scale bar (FIG. 30C) and 500 nm scale bar (FIG. 30D), highlighting their associated Microvesicles. FIGS. 30E-30F: bar graph detailing relative mRNA expression levels of various inflammation-related genes (FIG. 30E), while FIG. 30F shows cytokine levels between control and infected samples of human choroid plexus epithelial cells exposed to Borrelia sp.

FIGS. 31A-31B: FIG. 31A: Nervous system involvement may include Lyme meningitis, radicular pain, and involvement of the cranial nerves, resulting in a temporary and partial paralysis of one side of the face (Bell's palsy). Less common but more serious manifestations include encephalitis or cerebral vasculitis. Long-term sequelae may occur, and many patients experience fatigue, mood changes, difficulty sleeping, and deficits in short-term memory or ‘brain fog’. European patients with the late manifestation of acrodermatitis chronica atrophicans may experience chronic polyneuritis or neuropathy. FIG. 31B: The most common symptoms in children with neuroborreliosis.

FIGS. 32A-32B: Demonstrates how the duration of illness substantially affects the result of antibody-based tests FIG. 32A: rates of seropositivity C6 ELISA (VlsE and its C6 peptide as markers of antibody response in Lyme disease), WCS (whole cell sonicate) ELISA and 2-tier algorithm in relationship to disease presentation and time of sample (acute and convalescent). FIG. 32B: compares rates of seropositivity in relationship with duration of disease in patients with a single erythema migrans. EM: erythema migrans, LNB: Lyme neuroborreliosis, WCS: whole cell sonicate.

FIGS. 33A-33B: CD63 (FIG. 33A) and CD81 (FIG. 33B) extracted with urine extraction kit. This was performed to test: LD-related biomarkers and EVs may degrade over time, especially if incubation time or conditions are not optimal. These factors can reduce the detection of low-abundance proteins or peptides in mass spectrometry analysis.

FIGS. 34A-34B: A collapsible cup was developed to enable urine collection at the point-of-need, and capture of molecular analytes from urine. FIG. 34A: The Urigami collection devices were fabricated using a Cricut cutting machine, followed by manual gluing and folding to assemble the final structure. FIG. 34B: The volume of liquid contained in the open and collapsed Urigami cup is 50 mL and 0.5 mL, respectively. This was determined using DI water, graduated cylinder and micropipettes.

FIGS. 35A-35E: User acceptability of the Urigami urine collection cup. FIG. 35A: Overall user satisfaction ratings (N=210 participants) show that more than 80% of users rated the Urigami cup favorably. FIG. 35B: Urine collection process ratings show that over 90% of participants rated the cup ≥3. FIG. 35C: Device characteristics were evaluated for design and usability. User satisfaction with specific features of the Urigami prototype exceeded 90%. FIGS. 35D-35E: Acceptability among Nepalese participants (N=190). FIG>35D: for the urine collection process, over 92% of users rated the cup with a score of ≥3, confirming high satisfaction with the urine collection process. FIG. 35E: Device characteristics ratings show similar satisfaction levels, with more than 92% of users rating it favorably (score ≥3). A 5-point Likert scale was used: 1=least satisfied to 5=highly satisfied.

FIGS. 36A-36E: Urinary extracellular vesicles contain proteins deriving from multiple organ systems including the lungs. FIG. 36A: Urinary EV size analysis (Zeta view) showing average diameter. FIGS. 36B-36C: uEV proteome tissue-specific signature contributions: 30.75% from the liver, 28.09% from the lung, 16.95% from the kidney, and 12.11% from the brain in healthy individuals (FIG. 36B) and 28.30%, 18.87%, 17.69%, and 11.79% from the liver, brain, lung, and skin, respectively, in TB patients (FIG. 36C). FIGS. 36E-36F: Functional categorization of the uEV proteome revealed enrichment in proteins associated with immune defense in healthy (FIG. 36D) and TB (FIG. 36E) patients.

FIG. 37A-37E: The affinity net is efficient in capturing TB DNA in the EVs and in solution. FIG. 37A: uEVs isolated via centrifugation and affinity net from TB patients detecting presence of LAM. FIG. 37B: Affinity net captures uEV with a fast association kinetics. FIG. 37C: Affinity Net captures EV biomarker CD63, from uEVs. FIG. 37D: uEVs isolated via centrifugation from TB patients are positive for MtB RpoB gene. FIG. 37E: Affinity net captures urinary DNA directly from patients (detected using RpoB primers), thus suggesting RpoB gene is in the EVs.

FIG. 38: Table showing clinical and demographic characteristics of hospitalized, microbiologically TB positive patients (N=24).

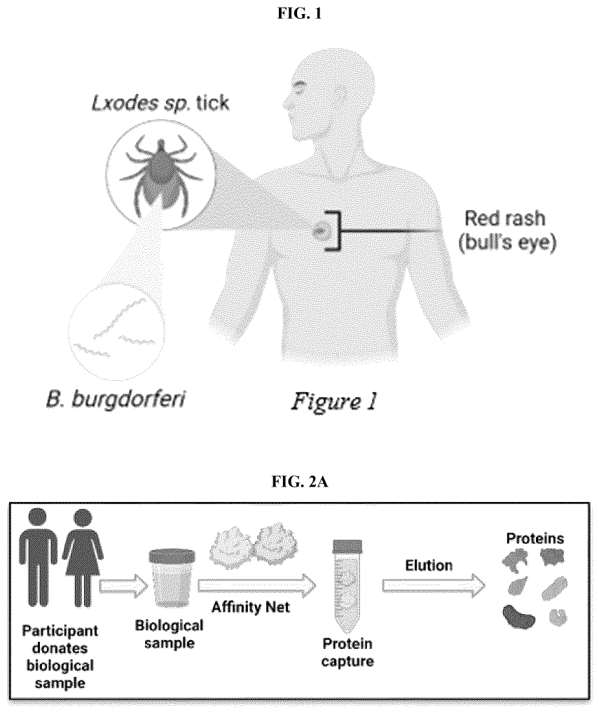

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### DESCRIPTION

Lyme Disease Diagnosis and/or Treatment and Neuroborreliosis Pathogenesis

Lyme disease (LD), caused by Borrelia burgdorferi, is a burgeoning public health concern with 42,743 confirmed and an estimated 300,000 underreported incidents annually in the USA. As a chronic, multisystemic vector-borne disease, LD has persisted in its global expansion, becoming a serious public health issue, particularly in low-to-middle-income countries (LMIC). Accurate, early-stage diagnostics for LD pose significant challenges due to variable clinical presentations, including nonspecific early symptoms like other conditions, the absence or failed detection of erythema migrants, insufficient sensitive tests, and ambiguous serological results. Additionally, Borrelia sp. continues to be harbored by both patients and animals post-antibiotic treatment. The complexity is further heightened by neuroborreliosis, a form of LD of unknown pathogenesis, leading to various neurological manifestations, often resulting in diagnostics delay or misdiagnosis.

Hence, there is a clinical need for an affordable and accessible diagnostic solution to democratize access to LD diagnostics and surveillance. In one aspect, the disclosure describes the development of a solid-based affinity enrichment technology to improve significantly (A) LD diagnostics and (B) understanding of neuroborreliosis. This technology captures LD-related biomarkers and Extracellular Vesicles (EVs) of eukaryotic origin in patient urine and Microvesicles (MVs) from Borrelia burgdorferi culture, holding the potential to enable a first-in-kind sensitive and accurate direct test for LD Notably, this approach can be particularly impactful in LMICs and underserved populations, where economic constraints often impede access to complex diagnostic procedures. This technology, “PolyDyeMesh” (PDM) and “DyGel Polyamide” (DGP), utilizes polyamide 6 functionalized with polymer/hydrogel and/or small molecule dyes for semi-specific affinity binding to solution-based bioanalytes to be further recovered into smaller volumes through affinity disruption. The resulting eluate can be used for downstream applications such as mass spectrometry and immunoassays, while the isolated MVs can be characterized and utilized to study the pathogenesis of neuroborreliosis.

In one aspect, the disclosure describes the development and characterization of PolyDyeMesh (PDM) and DyGel Polyamide (DGP)-containing dye molecules. The disclosure demonstrates that PDM and DGP can be successfully synthesized from/onto polyamide 6, with modified surface chemistry, yielding unique chemical properties optimal for capturing and recovering biomarkers from biofluids. Synthetic strategies will encompass the grafting of polyamide 6 with acrylamide-based hydrogel through free-radical polymerization and/or dye molecules via absorption or chemical reactions involving carboxyl or amine terminals. To characterize these novel materials, PDM and DGP will be analyzed using Fourier-transform infrared (FTIR) spectroscopy, mass spectrometry, and UV-Vis Spectrophotometry.

In another aspect, the disclosure describes the investigation of the biomarker sequestration capability of PolyDyeMesh and/or DyGel Polyamide. The disclosure demonstrates that PDM and/or DGP can effectively capture LD-related biomarkers and EVs from urine samples, enabling higher analytical sensitivity and specificity compared to the absence of enrichment. Patient urine samples will be collected and incubated with PDM and/or DGP, serving as a means to capture and concentrate LD-related biomarkers and/or EVs. The captured proteomic content will be analyzed using untargeted mass spectrometry and Western Blot analysis.

In another aspect, the disclosure describes the investigation of the role of Borrelia-derived Microvesicles in Neuroborreliosis Pathogenesis. The disclosure demonstrates that MVs from Borrelia sp. significantly influence the HMC3 human microglial cell line, suggesting a pivotal role in the inflammatory response associated with neuroborreliosis. PDM and/or DGP will be explored to isolate MVs from Borrelia sp. culture. MVs will be characterized by proteomic, genomic, and lipid content. The inflammatory responses to Borrelia-derived MVs will be explored by measuring NFκB activation and target transcription by Phospho-NFκB p62 by Western blot in nuclear extract and NFκB DNA-binding ELISA. Cytokines will be measured by RT-qPCR and human-specific high-sensitivity ELISA kits and Luminex in the cell lysates and culture media.

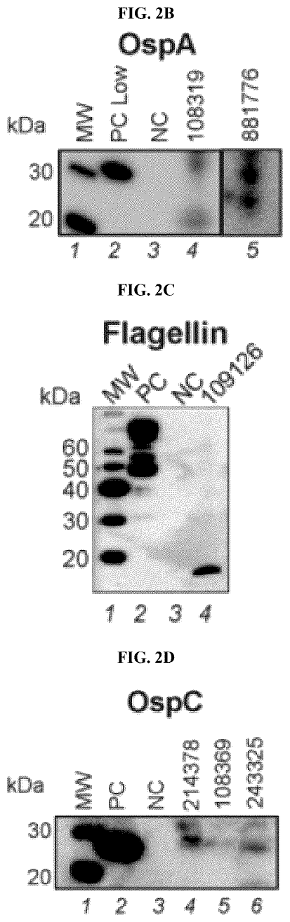

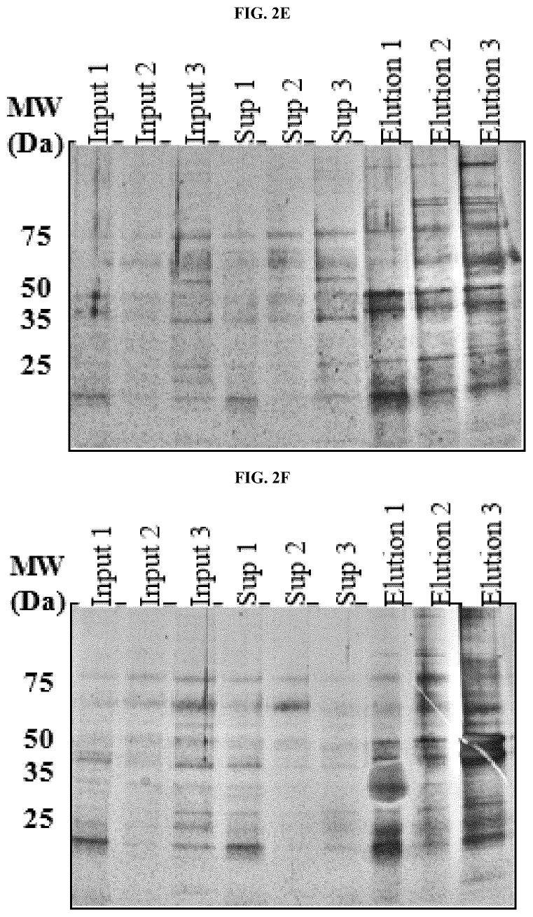

In one aspect, the disclosure describes an innovative methodology that employs solid-based, affinity-based materials PolyDyeMesh (PDM) and DyGel Polyamide (DGP). These advanced materials are conceptualized to capture and allow the study of critical biomolecular entities associated with Lyme disease directly from patient urine, a shift from traditional serological methods (FIG. 2A). This innovation is based on polyamide 6 fibers functionalized with polymer/hydrogel and/or small molecule dyes, which facilitates semi-specific affinity binding to solution-based bioanalytes, allowing for their subsequent recovery into more concentrated volumes through affinity disruption (FIG. 2A). Previous studies have shown that urine, a noninvasively collected biofluid, contains an abundance of Borrelia-derived peptides/proteins (FIGS. 2B-2D). The challenge, however, lies in the low concentrations of these biomarkers, making detection thresholds critical. Further, peptides/proteins present in urine are susceptible to degradation over time.

By incorporating PDM and DGO, these biomarkers can be concentrated and preserved. Urinary biomarkers can be captured semi-selectively through PDM and DGP baited with small molecular dyes. These biomarkers are detached using elution buffers (FIG. 2E), producing an eluate suitable for multiple downstream applications such as Western Blot and mass spectrometry. Further, the same applies to EV capture. Previous studies have shown the versatility of NIPA (N-isopropyl acrylamide) hydrogels in capturing and releasing molecular entities upon structural modifications caused by temperature changes. DGP (polyamide 6 grafted with NIPA-derived hydrogels) can capture EVs from patients' urine, which can be released by transferring the DGP into a smaller volume of a cold solution. Restructuration of the polymer can be sufficient to promote the elusion of intact EVs.

Tuberculosis (TB) Diagnosis and/or Treatment

Tuberculosis (TB) is a leading health issue worldwide. In 2023, an estimated 10.8 million people globally, including 1 million children and adolescents, fell ill with TB, and 1.5 million died from it. Individuals with active TB are treated with months-long multidrug antibiotic therapy, which is successful in susceptible tuberculosis cases that maintain continuity of treatment. TB therapy outcomes are severely affected by patient access to care, health knowledge, and socioeconomic status. Timely diagnosis of TB is essential for efficient patient care in terms of both symptom reduction in affected patients and containment of contagion spread.

Although highly accurate, the standard of care approaches to TB diagnosis, such as microbial culture and nucleic acid amplification-based tests, suffer from limitations in affordability and implementation, particularly in underserved settings. The need to utilize sputum is a limiting factor in large patient populations, including children, the elderly, and immunocompromised individuals.

Urine is an attractive biofluid for diagnostics, given its large volume and noninvasive nature. It has been shown that the use of nanotechnology helped detect lipoarabinomannan (LAM) in the urine of HIV-negative, TB-positive patients. The concentration of LAM ranged from 5 to 5000 picogram/mL. Although the reliable detection of Mtb-derived molecules such as LAM in the urine of pulmonary tuberculosis patients has been proven in research settings, the clinical implementation of a lateral flow immunoassay test for Mtb LAM is recommended only for people living with HIV due to sensitivity limitations. In patients with advanced immunosuppression, higher Mtb burdens are often observed, which in turn leads to increased concentrations of LAM in the urine.

Urine extracellular vesicles (uEVs) are emerging as a promising fraction to detect molecular patterns of diagnostic utility. Extracellular vesicles (EVs) are nano-sized lipid bound structures that are secreted by every cell type across prokaryotes and eukaryotes. They aid in cell-to-cell communication by delivering biomolecules that influence tissue dynamics, immune responses, cell behavior, and disease states. Mtb-infected cells secrete EVs that were shown to contain Mtb molecular payload, including lipoproteins, and nucleic acids. Mtb LAM was present in EVs secreted by infected eukaryotic cells and in EVs isolated from the blood and urine of TB patients.

The RpoB gene, which encodes the P-subunit of RNA polymerase, has been validated as a molecular target for pulmonary TB detection and to guide appropriate therapy. RpoB detection is usually accomplished in sputum, but other bodily fluids have also been investigated. In extrapulmonary TB, where the bacterial load is often low and sputum or tissue specimens are difficult to obtain, urine-based PCR assays targeting RpoB (e.g., using concentrated urine sediments or extracellular vesicle preparations) have been shown to detect Mtb DNA and improve diagnostic sensitivity.

While urine is gaining interest as a non-invasive sample for molecular diagnostics of systemic (non-urinary tract) diseases, it presents certain limitations. Many systemic illnesses release only trace amounts of molecular markers into urine, making detection challenging without highly sensitive analytical techniques. Collecting urine outside clinical environments can lead to contamination which in turn can lead to false positive or negative results. The composition of urine varies from one person to another as it depends on diet, kidney function, hydration and circadian rhythms. Urinary analytes are quickly degraded by endogenous and exogenous enzymes unless urine is promptly processed or preserved.

Sterile urine collection cups (e.g., the BD vacutainer complete urine collection system) are commonly used in clinical diagnostics to support urine culture, dipstick testing and microscopy analysis. However, current urine collection cups are not optimized for molecular testing, and present challenges for patients and laboratory personnel alike, including spillage, contamination, rupture, large volumes, and specific handling requirements such as refrigeration. Without proper stabilization, urinary nucleic acids are susceptible to degradation by endogenous and exogenous nucleases. The chemical additives of certain BD vacutainer tubes can disrupt urinalysis assays, and are not compatible with molecular biomarker systems unless augmented with targeted stabilizers and specialized transport conditions.

In one aspect, the disclosure describes the exploration of a novel collection and shipment technology that could overcome these hurdles and maximize the utility of urine as diagnostic fluid. Exemplary devices of the disclosure have two attributes: (1) they can be folded flat to be shipped with a regular mail envelope, and (2) they contain an affinity material (affinity net) that captures and sequesters bioanalytes that can be analyzed with standard molecular biology techniques. Described herein are the results of user acceptability surveys conducted in TB endemic areas, demonstrate the volumetric concentration capabilities of the foldable device, and apply the affinity net to enable the measurement of Mtb marker such as LAM and the RpoB gene in the uEVs of TB patients.

### General

In certain embodiments, the disclosure provides an efficient, accurate, and time-saving process of capturing and analyzing small molecules and peptides from biofluids and environmental samples. In certain embodiments, the disclosure provides the possibility of automation for the process, hence enhancing the speed of the process and analysis.

In certain embodiments, the affinity matrix of the disclosure provides the ability to preserve the molecular integrity and cell viability of the analytes. In certain embodiments, affinity matrix and methods of use thereof of the disclosure involves a biomaterial design based on affinity, an emphasis on urine as a non-invasive biofluid to characterize host-pathogen interactions in a human host, and focuses on Borrelia-derived microvesicles (MV) as a mediator of neuroinflammation characteristic of neuroborreliosis.

In certain embodiments, the biomaterial employs a solid-based, affinity-based materials PolyDyeMesh (PDM) and DyGel Polyamide (DGP). These advanced materials are conceptualized to capture and allow the study of critical biomolecular entities associated with Lyme disease directly from human urine, a shift from traditional serological methods. In certain embodiments, polyamide 6 fibers functionalized with polymer/hydrogel and/or small molecule dyes, which facilitates semi-specific affinity binding to solution-based bioanalytes, allowing for their subsequent recovery into more concentrated volumes through affinity disruption. The challenge, however, lies in the low concentrations of these biomarkers, making detection thresholds critical. Additionally, peptides/proteins present in urine are susceptible to degradation over time.

In one aspect, the disclosure addresses these challenges by incorporating PDM and DGP, permitting these biomarkers to be concentrated and preserved. Urinary biomarkers may be captured semi-selectively through PDM and DGP baited with small molecular dyes. These biomarkers may be detached using elution buffers producing an eluate suitable for multiple downstream applications such as Western Blot and mass spectrometry. Further, the same may apply to EV capture. Previous studies have shown the versatility of NIPA (N-isopropyl acrylamide) hydrogels in capturing and releasing molecular entities upon structural modifications caused by temperature changes. DGP (e.g., polyamide 6 grafted with NIPA-derived hydrogels) may capture EVs from patients' urine, which may be released by transferring the DGP into a smaller volume of a cold solution. Restructuration of the polymer may be sufficient to promote the elusion of intact EVs.

In certain embodiments, urine represents a window into host-pathogen interactions. An extensive collection of urine samples may help segregate patients and show the applicability of the previously described approach. In certain embodiments, Borrelia-derived MVs act as mediators of neuroinflammation. Similarly, MVs from Borrelia culture may be (and have been) isolated and studied, refining their proteomics and genomic profiles. These profiles are relatively uncharted in the context of Lyme disease. Studies have previously hypothesized that Lyme's neuro-inflammatory nature directly results from bacterial interactions. By introducing MVs to the HMC3 cell line, this disclosure expands the horizons to scrutinize the potential impact of MVs on human microglial cells, emphasizing an underlying mechanism associated with the neuro-inflammatory process linked to neuroborreliosis. These results help understand pathogen-host interaction at the microvesicle level. Furthermore, assessing inflammatory cascade through NFκB signaling and cytokine production provides a detailed map and comprehensive understanding of inflammatory mediators in response to MVs.

It has been shown that enrichment of Toxoplasmosis urine-derived antigens, enhances their detectability via mass spectrometry analysis. This is further exemplified using glass wool affinity fibers to capture urine-derived DNA, and the Urigami cup, a patient-friendly collection device integrated with glass wool to streamline the process of analyte collection for further proteomics analysis.

Nylon-based technologies further underscore the technical proficiency in affinity-binding technologies. Nylon dyed with Sudan Black B exemplifies the molecular interactions driving the dye-nylon affinity process. Gel electrophoresis provides evidence of the efficacy in capturing all the urine-derived biomarkers and releasing them into a smaller, more concentrated solution. Protein enhancement may be observed by comparing lanes with urine subjected to enrichment (Elution 1-3) against those without (Input 1-3). This validates the assay and emphasizes the technology's ability to enhance the sensitivity and specificity of Lyme-derived biomarker detection.

### Definitions

The term “about” as used herein can allow for a degree of variability in a value or range, for example, within 10%, within 5%, or within 1% of a stated value or of a stated limit of a range, and includes the exact stated value or range.

The term “about” as used herein can allow for a degree of variability in a value or range, for example, within 10%, within 5%, or within 1% of a stated value or of a stated limit of a range, and includes the exact stated value or range.

The term “adsorbed” as used herein refers to a condition in which a substance (e.g., an analyte) is bound to the surface of a material (e.g., a nylon fiber) through physical, chemical, or physicochemical interactions. Such interactions may include hydrogen bonding, ionic interactions, hydrophobic interactions, van der Waals forces, or coordination bonds. Adsorption may occur on the external surface of a material or within pores, interstices, or surface irregularities, and does not require that the analyte penetrate into or be absorbed within the bulk of the material.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkylene” or “alkylenyl” as used herein refers to a bivalent saturated aliphatic radical (e.g., —CH₂—, —CH₂CH₂—, and —CH₂CH₂CH₂—, inter alia). In certain embodiments, the term may be regarded as a moiety derived from an alkene by opening of the double bond or from an alkane by removal of two hydrogen atoms from the same (e.g., —CH₂—) different (e.g., —CH₂CH₂—) carbon atoms.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C—CH, —C≡C(CH₃), —C≡C(CH₂CH₃), —CH₂C≡CH, —CH₂C≡C(CH₃), and —CH₂C≡C(CH₂CH₃) among others.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3—, 4-, 5-, or 6-positions of the phenyl ring, or a naphthyl group substituted at any one or more of 2- to 8-positions thereof.

The term “biocompatible” as used herein refers to a material that is substantially non-toxic in the in vivo environment of its intended use, and that is neither substantially chemically degraded nor substantially rejected by the patient's physiological system (i.e., is non-antigenic).

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups.

The term “cycloalkylene” or “cycloalkylenyl” as used herein refers to a bivalent saturated cycloalkyl radical

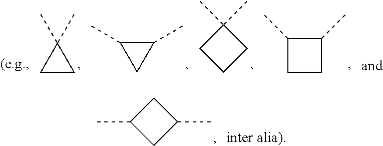

In certain embodiments, the term may be regarded as a product of removal of two hydrogen atoms from the corresponding cycloalkane (e.g., cyclobutyl) by removal of two hydrogen atoms from the same

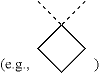

different

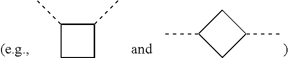

carbon atoms.

The term “disposed on” as used herein indicates that an object or element is coupled to, deposited on, or grown on a surface of another object or element.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

The term “fibrous” as used herein refers to a material characterized by the presence of fibers, i.e., elongated bodies, strands, or filaments having a length that is substantially greater than their thickness. The fibers may be synthetic (e.g., nylon, polyester, polypropylene, polyethylene, polyacrylonitrile), semi-synthetic (e.g., rayon, viscose), or natural (e.g., cellulose, collagen, silk, cotton, wool). The fibers may be continuous or discontinuous, woven or non-woven, aligned or randomly oriented, and may be present as individual strands, bundles, mats, meshes, or fabrics.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl) , indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylalkyl” as used herein refers to alkyl groups as defined herein in which a hydrogen or carbon bond of an alkyl group is replaced with a bond to a heteroaryl group as defined herein.

The term “heteroarylene” or “heteroarylenyl” as used herein refers to a bivalent heteroaryl radical (e.g., 2,4-pyridylene). In certain embodiments, the term may be regarded as a divalent radical formed by the removal of two hydrogen atoms from one or more rings of a heteroaryl moiety, wherein the hydrogen atoms may be removed from the same or different rings, preferably the same ring.

The term “heterocycloalkyl” as used herein refers to an aliphatic, partially unsaturated or fully saturated, 3- to 14-membered ring system, including single rings of 3 to 8 atoms and bi- and tricyclic ring systems where at least one of the carbon atoms of the ring is replaced with a heteroatom such as, but not limited to, nitrogen, oxygen, sulfur, or phosphorus. A heterocycloalkyl can include one to four heteroatoms independently selected from oxygen, nitrogen, and sulfur, wherein a nitrogen and sulfur heteroatom optionally can be oxidized and a nitrogen heteroatom can be optionally substituted. Representative heterocycloalkyl groups include, but are not limited, to the following exemplary groups: pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. The term heterocycloalkyl group can also be a C₂ heterocycloalkyl, C₂-C₃ heterocycloalkyl, C₂-C₄ heterocycloalkyl, C₂-C₅ heterocycloalkyl, C₂-C₆ heterocycloalkyl, C₂-C₇ heterocycloalkyl, C₂-C₈ heterocycloalkyl, C₂-C₉ heterocycloalkyl, C₂-C₁₀ heterocycloalkyl, C₂-C₁₁ heterocycloalkyl, and the like, up to and including a C₂-C₁₄ heterocycloalkyl. For example, a C₂ heterocycloalkyl comprises a group which has two carbon atoms and at least one heteroatom, including, but not limited to, aziridinyl, diazetidinyl, oxiranyl, thiiranyl, and the like. Alternatively, for example, a C₅ heterocycloalkyl comprises a group which has five carbon atoms and at least one heteroatom, including, but not limited to, piperidinyl, tetrahydropyranyl, tetrahydrothiopyranyl, diazepanyl, and the like. It is understood that a heterocycloalkyl group may be bound either through a heteroatom in the ring, where chemically possible, or one of carbons comprising the heterocycloalkyl ring. The heterocycloalkyl group can be substituted or unsubstituted.

The term “heterocycloalkylene” or “heterocycloalkylenyl” as used herein refers to a bivalent saturated cycloalkyl radical

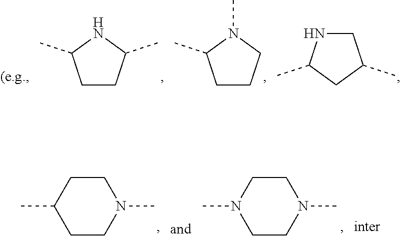

In certain embodiments, the term may be regarded as a product of removal of two hydrogen atoms from the corresponding heterocycloalkane (e.g., piperidine) by removal of two hydrogen atoms from the same

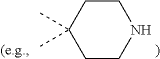

different

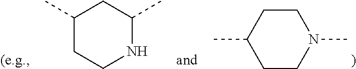

carbon atom(s) and/or heteroatom(s).

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound or compounds as described herein (alone or in combination with another pharmaceutical agent), to a patient, or application or administration of a therapeutic agent to a patient, or an isolated tissue or cell line from a patient (e.g., for diagnosis or ex vivo applications), who has a condition contemplated herein or a symptom of a condition contemplated herein, with the purpose to cure, heal, alleviate, relieve, alter, remedy, ameliorate, improve or affect a condition contemplated herein, or the symptoms of a condition contemplated herein.

### Affinity Matrix Compositions

In one aspect, the disclosure provides an affinity matrix comprising:
a biocompatible fibrous material;
a hydrogel; and
an affinity label,
wherein the hydrogel is disposed on the biocompatible fibrous material, and
wherein the affinity label is associated with at least one of the biocompatible fibrous material and the hydrogel.

In certain embodiments, the biocompatible fibrous material comprises nylon. In certain embodiments, the nylon is a nylon sheet.

In certain embodiments, the nylon is nylon 6. In certain embodiments, the nylon is nylon 6,6. In certain embodiments, the nylon is nylon 4,6. In certain embodiments, the nylon is nylon 6,9. In certain embodiments, the nylon is nylon 6,10. In certain embodiments, the nylon is nylon 6,12. In certain embodiments, the nylon is nylon 11. In certain embodiments, the nylon is nylon 12.

In certain embodiments, the affinity label comprises a dye.

In certain embodiments, the dye is an acid dye. In certain embodiments, the dye is a basic dye. In certain embodiments, the dye is a reactive dye. In certain embodiments, the dye is a disperse dye. In certain embodiments, the dye is a direct dye. In certain embodiments, the dye is a mordant dye. In certain embodiments, the dye is a vat dye. In certain embodiments, the dye is a sulfur dye. In certain embodiments, the dye is an azo dye. In certain embodiments, the dye is an anthraquinone dye. In certain embodiments, the dye is a triphenylmethane dye. In certain embodiments, the dye is a phthalocyanine dye. In certain embodiments, the dye is a polymethine dye. In certain embodiments, the dye is an indigoid dye. In certain embodiments, the dye is a nitro dye. In certain embodiments, the dye is a fluorescent dye. In certain embodiments, the dye is a natural dye.

In certain embodiments, the dye is Acid Black 48. In certain embodiments, the dye is Acid Red 87. In certain embodiments, the dye is Acid Red 92. In certain embodiments, the dye is Acid Orange 50. In certain embodiments, the dye is Acid Fuchsin. In certain embodiments, the dye is Bismarck Brown. In certain embodiments, the dye is Cibacron Red. In certain embodiments, the dye is Cibacron Yellow. In certain embodiments, the dye is Crystal Violet. In certain embodiments, the dye is Safranin O. In certain embodiments, the dye is Methylene Blue. In certain embodiments, the dye is Pinacyanol Chloride. In certain embodiments, the dye is Fast Blue B+Naphthionic acid. In certain embodiments, the dye is Fast Blue B+Laurent Acid. In certain embodiments, the dye is Fast Blue B+Cleve Acid. In certain embodiments, the dye is Fast Blue B+Peri Acid. In certain embodiments, the dye is Alcian Blue Pyridine variant. In certain embodiments, the dye is Ni Phthalocyanine. In certain embodiments, the dye is Fe Phthalocyanine. In certain embodiments, the dye is Pinacyanol Chloride. In certain embodiments, the dye is Reactive Red 120. In certain embodiments, the dye is Reactive Blue 21. In certain embodiments, the dye is Remazol B Blue. In certain embodiments, the dye is Sudan I. In certain embodiments, the dye is Sudan IV. In certain embodiments, the dye is Sudan Black B. In certain embodiments, the dye is Oil Red O. In certain embodiments, the dye is Acid Black 48. In certain embodiments, the dye is Bismarck Brown Y. In certain embodiments, the dye is Alizarin Cyanin. In certain embodiments, the dye is Eosin B.

In certain embodiments, the hydrogel comprises a crosslinked polymer. In certain embodiments, the crosslinked polymer is a product of a polymerization reaction between:
at least one vinyl monomer; and
at least one divinyl crosslinker.

In certain embodiments, each vinyl monomer is independently a compound of formula (I):

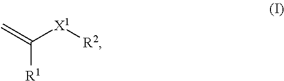

wherein:
   R¹ is selected from the group consisting of H, optionally substituted C₁-C₃ alkyl, and a halogen;
   R² is selected from the group consisting of N(R^{A})(R^{B}), OR^{A}, CN, NO₂, halogen, optionally substituted C₆-C₁₀ aryl, and optionally substituted C₂-C₁₀ heteroaryl;
   X¹ is selected from the group consisting of a bond, —C(R^{3a})(R^{3b})—, —C(═O)—, —P(═O)OR^{A}—, —S(═O)—, and —S(═O)₂—;
   R^{3a} and R^{3b} are each independently selected from the group consisting of H, optionally substituted C₁-C₃ alkyl, and a halogen; and
   each occurrence of R^{A} and R^{B} are each independently selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl, or R^{A} and R^{B} can combine with the nitrogen atom to which they are bound to form an optionally substituted C₂-C₈ heterocycloalkyl.

In certain embodiments, R¹ is H. In certain embodiments, R¹ is CH₃. In certain embodiments, R¹ is Cl.

In certain embodiments, R² is NH₂. In certain embodiments, R² is NHCH₃. In certain embodiments, R² is N(CH₃)₂. In certain embodiments, R² is OMe. In certain embodiments, R² is CN. In certain embodiments, R² is NO₂. In certain embodiments, R² is Cl. In certain embodiments, R² is phenyl.

In certain embodiments R^{3a} is H. In certain embodiments, R^{3b} is H. In certain embodiments R^{3a} is CH₃. In certain embodiments, R^{3b} is CH₃. In certain embodiments R^{3a} is F. In certain embodiments, R^{3b} is F.

In certain embodiments, X¹ is CH₂. In certain embodiments, X¹ is —C(═O)—. In certain embodiments, X¹ is —S(═O)—. In certain embodiments, X¹ is —S(═O)₂—. In certain embodiments, X¹ is —P(═O)(OH)—.

In certain embodiments, each vinyl monomer is independently selected from the group consisting of N-isopropylacrylamide, acrylamide, methacrylamide, vinyl sulfonic acid, allylamine, N-vinylpyrrolidone, acrylic acid, methacrylic acid, hydroxyethyl acrylate, hydroxyethyl methacrylate, vinyl acetate, styrene, 2-acrylamido-2-methyl-1-propanesulfonic acid, acrylonitrile, methacrylonitrile, vinyl imidazole, vinyl phosphonic acid, vinyl chloride, vinylidene chloride, glycidyl methacrylate, dimethylaminoethyl acrylate, dimethylaminoethyl methacrylate, and itaconic acid.

In certain embodiments, the vinyl monomer comprises N-isopropylacrylamide. In certain embodiments, the vinyl monomer comprises N-isopropylacrylamide and allylamine. In certain embodiments, the vinyl monomer comprises N-isopropylacrylamide and vinyl sulfonic acid. In certain embodiments, the vinyl monomer comprises acrylamide.

In certain embodiments, each divinyl monomer is independently a compound of formula (II):

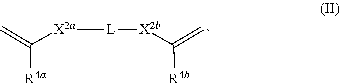

wherein:
   R^{4a} and R^{4b} are each independently selected from the group consisting of H, optionally substituted C₁-C₃ alkyl, and a halogen;
   X^{2a} and X^{2b} are each independently selected from the group consisting of a bond, —C(R^{5a})(R^{5b})—, —C(═O)—, —P(═O)OR^{C}—, —S(═O)—, and —S(═O)₂—;
   L is at least one selected from the group consisting of —N(R^{C})—, —O—, —C(═O)—, -(optionally substituted C₁-C₆ alkylenyl)-, -(optionally substituted C₁-C₆ heteroalkylenyl)-, -(optionally substituted C₃-C₈ cycloalkylenyl)-, -(optionally substituted C₂-C₈ heterocycloalkylenyl)-, -(optionally substituted C₆-C₁₀ arylenyl)-, and -(optionally substituted C₂-C₈ heteroarylenyl)-;
   R^{5a} and R^{5b} are each independently selected from the group consisting of H, optionally substituted C₁-C₃ alkyl, and a halogen; and
   each occurrence of R^{C} is independently selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl.

In certain embodiments, R^{4a} is H. In certain embodiments, R^{4a} is CH₃. In certain embodiments, R^{4a} is Cl. In certain embodiments, R^{4b} is H. In certain embodiments, R^{4b} is CH₃. In certain embodiments, R^{4b} is Cl.

In certain embodiments, L is —[(CH₂)₂O]—. In certain embodiments, L is —[(CH₂)₂O]₂—. In certain embodiments, L is —[(CH₂)₂O]₃—. In certain embodiments, L is —[(CH₂)₂O]₄—. In certain embodiments, L is —[(CH₂)₂O]₅—. In certain embodiments, L is —(CH₂)—. In certain embodiments, L is —(CH₂)₂—. In certain embodiments, L is —(CH₂)₃—. In certain embodiments, L is —(CH₂)₄—. In certain embodiments, L is —(CH₂)₅—. In certain embodiments, L is —O—. In certain embodiments, L is —C(═O)—. In certain embodiments, L is —S(═O)—. In certain embodiments, L is —S(═O)₂—.

In certain embodiments R^{5a} is H. In certain embodiments, R^{5b} is H. In certain embodiments R^{5a} is CH₃. In certain embodiments, R^{5b} is CH₃. In certain embodiments R^{5a} is F. In certain embodiments, R^{5b} is F.

In certain embodiments, X^{2a} is CH₂. In certain embodiments, X^{2a} is —C(═O)—. In certain embodiments, X^{2a} is —S(═O)—. In certain embodiments, X^{2a} is —S(═O)₂—. In certain embodiments, X^{2a} is —P(═O)(OH)—. In certain embodiments, X^{2b} is CH₂. In certain embodiments, X^{2b} is —C(═O)—. In certain embodiments, X^{2b} is —S(═O)—. In certain embodiments, X^{2b} is —S(═O)₂—. In certain embodiments, X^{2b} is —P(═O)(OH)—.

In certain embodiments, each divinyl crosslinker is independently selected from the group consisting of N,N′-methylenebis(acrylamide), N,N′-ethylenebis(acrylamide), ethylene glycol diacrylate, ethylene glycol dimethacrylate, diethylene glycol diacrylate, diethylene glycol dimethacrylate, polyethylene glycol diacrylate, polyethylene glycol dimethacrylate, glycerol dimethacrylate, neopentyl glycol diacrylate, neopentyl glycol dimethacrylate, divinylbenzene, diallyl phthalate, and diallyl carbonate.

In certain embodiments, the affinity matrix comprises about 0.005, 0.010, 0.015, 0.020, 0.025, 0.030, 0.035, 0.040, 0.045, or about 0.050 mmol dye per milligram fibrous material. In certain embodiments, the affinity matrix comprises about 0.0164 mmol dye per milligram fibrous material.

In some embodiment, the ratio of affinity label to fibrous material correlates to the size and thickness of the fibrous material, the identity of the affinity label, and/or the identity of the analyte for capture. In certain embodiments, the ratio of affinity label to fibrous material suitable for capture of a particular analyte decreases as a function of increasing surface area to volume ratio. In such embodiments, the relative amount of affinity label required for analyte capture decreases with increased surface area to volume ratio of the fibrous material. In certain embodiments, the surface area to volume ratio of the fibrous material increases as a function of decreasing thickness of the fibrous material. In certain embodiments, the surface area to volume ratio of the fibrous material increases as a function of increasing porosity. In certain embodiments, the porosity of the fibrous material is negligible.

In certain embodiments, the fibrous material comprises nylon. In certain embodiments, the nylon comprises a nylon sheet. In certain embodiments, the nylon sheet comprises nylon 6,6.

In certain embodiments, wherein the nylon sheet has a surface area to volume ratio of about 3.7 mm⁻¹ (e.g., nylon sheet having a length of about 5 mm, a width of about 5 mm, and a thickness of about 0.7 mm), the nylon comprises about 0.015 g of dye to about 1.5 g of dye (e.g., Sudan Black B). In certain embodiments, wherein the nylon sheet has a surface-area-to-volume ratio of about 3.7 mm⁻¹, the nylon comprises about 15, 20, 25, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or about 1000 mg of dye (e.g., Sudan Black B). In certain embodiments, wherein the nylon sheet has a surface-area-to-volume ratio of about 1.85 mm⁻¹ (e.g., nylon sheet having a length of about 5 mm, a width of about 5 mm, and a thickness of about 1.9 mm), the nylon comprises about 0.030 g of dye to about 3.0 g of dye (e.g., Sudan Black B). In certain embodiments, wherein the nylon sheet has a surface-area-to-volume ratio of about 1.85 mm⁻¹, the nylon comprises about 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, or about 2000 mg of dye (e.g., Sudan Black B).

In certain embodiments, the vinyl monomer and divinyl crosslinker have a molar ratio of about 100:1, 100:2, 100:3, 100:4, 100:5, 100:6, 100:7, 100:8, 100:9, or about 100:10. In certain embodiments, the vinyl monomer and divinyl crosslinker have a molar ratio of about 100:3.

In certain embodiments, the association of the dye with the fibrous material or hydrogel comprises a non-covalent bonding interaction (e.g., hydrogen bonding interaction, Van der Waals interaction, and/or ionic bonding interaction).

In certain embodiments, the association of the dye with the fibrous material or hydrogel comprises a covalent bond between the dye and the fibrous material or hydrogel.

### Methods

In another aspect, the disclosure provides a method for preparing the affinity matrix of the disclosure, the method comprising:
contacting a fibrous material sheet with an affinity label to provide an affinity labeled fibrous material;
placing the affinity labeled fibrous material in a sealable vessel; and
contacting the affinity labeled fibrous material with a monomer solution comprising a vinyl monomer, a divinyl crosslinker, and a radical initiator under an anaerobic condition.

In certain embodiments, the contacting occurs in the presence of a bidentate ligand. In certain embodiments, the bidentate ligand comprises tetramethylenediamine (TEMED).

In another aspect, the disclosure provides a method for separating an analyte from a sample, the method comprising:
contacting a liquid sample with the affinity matrix of the disclosure to provide an analyte-adsorbed affinity matrix and a supernatant;
separating the analyte-adsorbed affinity matrix and the supernatant; and
contacting the analyte-adsorbed affinity matrix with an eluting solution to provide an analyte solution.

In certain embodiments, the contacting occurs for a period of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 minutes. In certain embodiments, the contacting occurs for about 10 minutes. In certain embodiments, the contacting occurs for about 15 minutes. In certain embodiments, the contacting occurs for about 30 minutes.

In certain embodiments, the separating comprises decanting, pipetting off, or filtering the supernatant from the analyte-adsorbed affinity matrix.

In certain embodiments, the eluting solution comprises a buffer solution. In certain embodiments, the buffer solution comprises urea and sodium dodecyl sulfate (SDS) (e.g., 2M urea and 3.85% SDS).

In certain embodiments, the analyte solution is subjected to an analytical assay. In certain embodiments, the analytical assay comprises mass spectrometry. In certain embodiments, the analytical assay comprises spectrophotometry. In certain embodiments, the analytical assay comprises chromatography. In certain embodiments, the analytical assay comprises an immunoassay. In certain embodiments, the analytical assay comprises a nucleic acid amplification assay.

In certain embodiments, the liquid sample and affinity matrix have a ratio of about 500, 450, 400, 350, 300, 250, 200, 150, 100, or about 50 μL liquid sample/cm² affinity matrix.

In certain embodiments, the liquid sample comprises a liquid biological sample. In certain embodiments, the liquid sample comprises a liquid environmental sample.

In certain embodiments, the liquid biological sample comprises urine. In certain embodiments, the liquid biological sample comprises blood. In certain embodiments, the liquid biological sample comprises plasma. In certain embodiments, the liquid biological sample comprises serum. In certain embodiments, the liquid biological sample comprises saliva. In certain embodiments, the liquid biological sample comprises cerebrospinal fluid. In certain embodiments, the liquid biological sample comprises vaginal fluid. In certain embodiments, the liquid biological sample comprises semen. In certain embodiments, the liquid biological sample comprises a cell culture medium.

In certain embodiments, the liquid environmental sample comprises wastewater. In certain embodiments, the liquid environmental sample comprises surface water. In certain embodiments, the liquid environmental sample comprises groundwater. In certain embodiments, the liquid environmental sample comprises river water. In certain embodiments, the liquid environmental sample comprises lake water. In certain embodiments, the liquid environmental sample comprises ocean water. In certain embodiments, the liquid environmental sample comprises industrial effluent.

In certain embodiments, the analyte is a protein. In certain embodiments, the analyte is a peptide. In certain embodiments, the analyte is a nucleic acid. In certain embodiments, the analyte is a carbohydrate. In certain embodiments, the analyte is a lipid. In certain embodiments, the analyte is lipid particle. In certain embodiments, the analyte is a small molecule. In certain embodiments, the lipid particle comprises a urinary extracellular vesicle (uEV).

In certain embodiments, the analyte is a biomarker of a pathogen or a subject infected with the pathogen. In certain embodiments, the biomarker comprises lipoarabinomannan (LAM), Mycobacterial (Mtb) DNA/RNA fragments, Mtb proteins, Borrelia DNA/RNA fragments, Borrelia outer surface proteins (OspA, OspC, OspE, RimM, and FliM), PGM (gpmA), MutL, HDDP, OEPF, and L20.

In certain embodiments, the biomarker comprises a urinary cytokine or chemokine. In certain embodiments, the biomarker is selected from the group consisting of CD9, CD81, CD59, CD44, CD59, CD63, tetraspanin-1, MIF, LAMP2, IGLL5, MAN1A1, HLA-Cw1α, HLA-DRB1-4 R (isoform X2), and CD14.

In certain embodiments, the pathogen is Borrelia burgdorferi or Mycobacterium tuberculosis.

In another aspect, the disclosure provides a method for diagnosing a disease or disorder in a subject, the method comprising:
contacting a liquid biological sample obtained from the subject with the affinity matrix of the disclosure to provide an analyte-adsorbed affinity matrix and a supernatant; separating the analyte-adsorbed affinity matrix and the supernatant;
contacting the analyte-adsorbed affinity matrix with an eluting solution to provide an analyte solution; and
detecting a biomarker associated with the disease or disorder in the analyte solution.

In certain embodiments, the contacting occurs for a period of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 minutes. In certain embodiments, the contacting occurs for about 10 minutes. In certain embodiments, the contacting occurs for about 15 minutes. In certain embodiments, the contacting occurs for about 30 minutes.

In certain embodiments, the separating comprises decanting, pipetting off, or filtering the supernatant from the analyte-adsorbed affinity matrix.

In certain embodiments, the eluting solution comprises a buffer solution. In certain embodiments, the buffer solution comprises urea and sodium dodecyl sulfate (SDS) (e.g., 2M urea and 3.85% SDS).

In certain embodiments, the analyte solution is subjected to an analytical assay. In certain embodiments, the analytical assay comprises mass spectrometry. In certain embodiments, the analytical assay comprises spectrophotometry. In certain embodiments, the analytical assay comprises chromatography. In certain embodiments, the analytical assay comprises an immunoassay. In certain embodiments, the analytical assay comprises a nucleic acid amplification assay.

In certain embodiments, the liquid sample and affinity matrix have a ratio of about 500, 450, 400, 350, 300, 250, 200, 150, 100, or about 50 μL liquid sample/cm² affinity matrix.

In certain embodiments, the liquid biological sample comprises urine. In certain embodiments, the liquid biological sample comprises blood. In certain embodiments, the liquid biological sample comprises plasma. In certain embodiments, the liquid biological sample comprises serum. In certain embodiments, the liquid biological sample comprises saliva. In certain embodiments, the liquid biological sample comprises cerebrospinal fluid. In certain embodiments, the liquid biological sample comprises vaginal fluid. In certain embodiments, the liquid biological sample comprises semen. In certain embodiments, the liquid biological sample comprises a cell culture medium.

In certain embodiments, the analyte is a protein. In certain embodiments, the analyte is a peptide. In certain embodiments, the analyte is a nucleic acid. In certain embodiments, the analyte is a carbohydrate. In certain embodiments, the analyte is a lipid. In certain embodiments, the analyte is lipid particle. In certain embodiments, the analyte is a small molecule. In certain embodiments, the lipid particle comprises a urinary extracellular vesicle (uEV).

In certain embodiments, the analyte is a biomarker of a pathogen or a subject infected with the pathogen.

In certain embodiments, the pathogen is a bacterium, a virus, a fungus, or a protozoan.

In other embodiments, the pathogen is a prion.

In certain embodiments, the virus a coronavirus. In certain embodiments, the coronavirus is SARS-CoV-1, SARS-CoV-2, MERS-CoV, HCoV-OC43, HCoV-229E, HCoV-NL63, or HCoV-HKU1. In certain embodiments, the biomarker comprises a spike (S) protein, nucleocapsid (N) protein, membrane (M) protein, or envelope (E) protein of a coronavirus, or a host-derived cytokine or antibody responsive to a coronavirus antigen.

In certain embodiments, the pathogen is an influenza virus. In certain embodiments, the influenza virus is Influenza A (e.g., H1N1, H1N2, H3N2, H5N1, H7N9) or Influenza B. In certain embodiments, the Influenza A virus causes swine flu or avian flu. In certain embodiments, the biomarker comprises a hemagglutinin (HA) protein, neuraminidase (NA) protein, nucleoprotein (NP), or matrix protein (M1/M2) of an influenza virus, or a host-derived cytokine or antibody responsive to influenza antigen.

In certain embodiments, the pathogen is a retrovirus. In certain embodiments, the retrovirus is Human Immunodeficiency Virus (e.g., HIV-1 or HIV-2). In certain embodiments, the biomarker comprises gp120, gp41, p24, or a host-derived cytokine or antibody responsive to an HIV antigen.

In certain embodiments, the pathogen is an orthopoxvirus. In certain embodiments, the orthopoxvirus is Monkeypox virus (mpox) or Vaccinia virus. In certain embodiments, the biomarker comprises a viral surface protein (e.g., A27L or B5R), or envelope antigen of an orthopoxvirus, or a host-derived cytokine or antibody responsive to an orthopoxvirus antigen.

In certain embodiments, the pathogen is a paramyxovirus (e.g., measles virus, mumps virus, parainfluenza virus, or respiratory syncytial virus (RSV)). In certain embodiments, the biomarker comprises a fusion (F) protein, nucleoprotein (N), or hemagglutinin-neuraminidase (HN) protein of a paramyxovirus, or a host-derived cytokine or antibody responsive to a paramyxovirus antigen.

In certain embodiments, the pathogen is a flavivirus (e.g., dengue virus, Zika virus, West Nile virus, yellow fever virus, or Japanese encephalitis virus). In certain embodiments, the biomarker comprises an envelope (E) or nonstructural (NS1) protein of a flavivirus, or a host-derived cytokine or antibody responsive to a flavivirus antigen.

In certain embodiments, certain embodiments, the pathogen is a bacterium. In certain embodiments, the bacterium is selected from Mycobacterium tuberculosis, Borrelia burgdorferi (Lyme disease), Streptococcus pneumoniae, Staphylococcus aureus, Escherichia coli, Salmonella enterica, or Clostridium difficile. In certain embodiments, the biomarker comprises a cell wall antigen, surface polysaccharide, toxin, or enzymatic product of a bacterium, or a host-derived cytokine or antibody responsive to a bacterial antigen.

In certain embodiments, the biomarker comprises lipoarabinomannan (LAM), Mycobacterial (Mtb) DNA/RNA fragments, Mtb proteins, Borrelia DNA/RNA fragments, Borrelia outer surface proteins (OspA, OspC, OspE, RimM, and FliM), PGM (gpmA), MutL, HDDP, OEPF, and L20.

In certain embodiments, the pathogen is Borrelia burgdorferi or Mycobacterium tuberculosis.

In certain embodiments, the pathogen is a protozoan, such as Plasmodium spp. (malaria) or Toxoplasma gondii. In certain embodiments, the biomarker comprises a secreted antigen, metabolite, or host immune biomarker indicative of infection by the protozoan.

In certain embodiments, the pathogen is a fungus, such as Candida albicans or Aspergillus fumigatus. In certain embodiments, the biomarker comprises a secreted antigen, metabolite, or host immune biomarker indicative of infection by the fungus.

In certain embodiments, the biomarker comprises a urinary cytokine or chemokine. In certain embodiments, the biomarker is selected from the group consisting of CD9, CD81, CD59, CD44, CD59, CD63, tetraspanin-1, MIF, LAMP2, IGLL5, MAN1A1, HLA-Cw1α, HLA-DRB1-4 β(isoform X2), and CD14.

In another aspect, the disclosure provides a method for treating a disease or disorder in a subject, the method comprising:
contacting a liquid biological sample obtained from the subject with the affinity matrix of the disclosure to provide an analyte-adsorbed affinity matrix and a supernatant;
separating the analyte-adsorbed affinity matrix and the supernatant;
contacting the analyte-adsorbed affinity matrix with an eluting solution to provide an analyte solution;
detecting a biomarker associated with the disease or disorder in the analyte solution; and
administering to the subject a therapeutic agent effective to treat the disease or disorder.

In certain embodiments, the disease or disorder comprises an infection with a pathogen.

In certain embodiments, the analyte solution comprises a biomarker of the pathogen or the subject.

In certain embodiments, infection comprises a bacterial infection. In certain embodiments, the bacterial infection comprises borreliosis (e.g., Lyme disease and neuroborreliosis) or tuberculosis.

In certain embodiments, the contacting occurs for a period of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 minutes. In certain embodiments, the contacting occurs for about 10 minutes. In certain embodiments, the contacting occurs for about 15 minutes. In certain embodiments, the contacting occurs for about 30 minutes.

In certain embodiments, the separating comprises decanting, pipetting off, or filtering the supernatant from the analyte-adsorbed affinity matrix.

In certain embodiments, the eluting solution comprises a buffer solution. In certain embodiments, the buffer solution comprises urea and sodium dodecyl sulfate (SDS) (e.g., 2M urea and 3.85% SDS).

In certain embodiments, the analyte solution is subjected to an analytical assay. In certain embodiments, the analytical assay comprises mass spectrometry. In certain embodiments, the analytical assay comprises spectrophotometry. In certain embodiments, the analytical assay comprises chromatography. In certain embodiments, the analytical assay comprises an immunoassay. In certain embodiments, the analytical assay comprises a nucleic acid amplification assay.

In certain embodiments, the liquid sample and affinity matrix have a ratio of about 500, 450, 400, 350, 300, 250, 200, 150, 100, or about 50 μL liquid sample/cm² affinity matrix.

In certain embodiments, the liquid biological sample comprises urine. In certain embodiments, the liquid biological sample comprises blood. In certain embodiments, the liquid biological sample comprises plasma. In certain embodiments, the liquid biological sample comprises serum. In certain embodiments, the liquid biological sample comprises saliva. In certain embodiments, the liquid biological sample comprises cerebrospinal fluid. In certain embodiments, the liquid biological sample comprises vaginal fluid. In certain embodiments, the liquid biological sample comprises semen. In certain embodiments, the liquid biological sample comprises a cell culture medium.

In certain embodiments, the analyte is a protein. In certain embodiments, the analyte is a peptide. In certain embodiments, the analyte is a nucleic acid. In certain embodiments, the analyte is a carbohydrate. In certain embodiments, the analyte is a lipid. In certain embodiments, the analyte is lipid particle. In certain embodiments, the analyte is a small molecule. In certain embodiments, the lipid particle comprises a urinary extracellular vesicle (uEV).

In certain embodiments, the analyte is a biomarker of a pathogen or a subject infected with the pathogen. In certain embodiments, the biomarker comprises lipoarabinomannan (LAM), Mycobacterial (Mtb) DNA/RNA fragments, Mtb proteins, Borrelia DNA/RNA fragments, Borrelia outer surface proteins (OspA, OspC, OspE, RimM, and FliM), PGM (gpmA), MutL, HDDP, OEPF, and L20.

In certain embodiments, the biomarker comprises a urinary cytokine or chemokine. In certain embodiments, the biomarker is selected from the group consisting of CD9, CD81, CD59, CD44, CD59, CD63, tetraspanin-1, MIF, LAMP2, IGLL5, MAN1A1, HLA-Cw1α, HLA-DRB1-4 R (isoform X2), and CD14.

In certain embodiments, the pathogen is Borrelia burgdorferi or Mycobacterium tuberculosis.

### Kits

In another aspect, the disclosure provides a kit comprising:
the affinity matrix of the disclosure;
a liquid collection device; and
instructions for preparing and storing an analyte-adsorbed affinity matrix in the liquid collection device.

In certain embodiments, the liquid collection device is collapsible.

In certain embodiments, the liquid collection device comprises a cup. In certain embodiments, the liquid collection device comprise a polypropylene sheet.

In certain embodiments, the kit further comprises an envelope configured to store the liquid collection device comprising the analyte-adsorbed affinity matrix.

### EXAMPLES

Various embodiments of the present application can be better understood by reference to the following Examples which are offered by way of illustration. The scope of the present application is not limited to the Examples given herein.

### Materials and Methods

### Preparation of PDM for Dyeing

Nylon 6 fibers will be washed thoroughly with Alconox 0.1%. Two washes of 30 min in the rotators will be performed, followed by four washes with MiliQ water 1 hr. each. Fibers will be dried at 30° C. overnight to eliminate any excess of water.

Preparation of PDM with Reactive Dyes

0.24 gr of nylon fibers will be placed in the oven at 60° C. to promote fiber expansion. Meanwhile, the dye bath will be prepared as follows: 12 mL of MiliQ water will be placed in a dye bath container, and the dispersing agent, sodium 1-naphthalenesulfonate, will be added to achieve 1 g/l final concentration and mixed until complete dissolution. pH will be adjusted to 5 using acetic acid. The dye will be weighted and incorporated into the dye bath to achieve a 1:12 dye: bath ratio. Nylon fibers will be incorporated into the dye bath and the oven. The dyeing process will start at 40° C. and slowly rise to 95° C. at 5° C./min and is continued for 1 hour when exhaustion is completed. The oven will be turned off, and the tubes will cool down to room temperature before transferring the nylon thread into the tube containing the fixation solution. The fixing solution will be prepared using sodium carbonate (3 g/L) at pH 10. 30 mL of fixing solution will be placed into a container, and nylon fibers will be transferred. Fixation will occur in the rotator at room temperature for 45 minutes. Next, fibers will be washed with four washes of MiliQ water for 1 hr. each. Fibers will then be placed in a glass container to dry in the oven at 30° C.

Preparation of PDM with Direct and Disperse Dyes

The same protocol as Reactive dyes is applied with a slight modification: sodium chloride will be added to the dye bath to achieve a 50 g/l final concentration.

Preparation of PDM with Basic Dyes

The same protocol as Reactive dyes is applied with a slight modification: sodium chloride will be added to the dye bath to achieve a 2% final concentration and pH 5.5. The dye will be weighted and incorporated into the bath to achieve 3% W/V. After dye incorporation, fibers will be washed with 20% W/V NaCl for 30 min in the rotator, twice, and then with four washes of MiliQ water for 1 hr. each. Fibers will then be placed in a glass container to dry in the oven at 30° C.

Preparation of PDM with Acid Dyes

The same protocol as Basic dyes is applied with a slight modification: sodium sulfate will be added to achieve 10% final concentration and mixed until complete dissolution. pH will be adjusted to 4.5 using acetic acid. After dye incorporation, fibers will be washed with Alconox 0.1% twice for 30 min, then with four washes of MiliQ water for 1 hr. each. Fibers will then be placed in a glass container to dry in the oven at 30° C.

Quality control for PDM will be performed through dye-bound quantification. 6 mg of PDM will be dissolved in triplicate in 1000p1 of 22% W/V CaCl2)/MeOH solution. The incorporated dye will be quantified using a calibration curve constructed to measure absorbance values over the concentration of a solution of free dye dissolved in 22% W/V CaCl2)/MeOH.

Preparation of Acrylamide-Based Hydrogel onto Nylon 6—DGP

A solution containing 20% % T (total concentration of monomers) of NIPA (n-isopropyl acrylamide), BIS (n n′-methylene-bis-acrylamide, crosslinker), and/or VSA (vinylsulfonic acid), and/or A.A. (allylamine) will be prepared in a sealed tube. % C (percentage crosslinker) will be adjusted upon application requirements. Nitrogen degassing of the monomer mixture will be performed for 1 hour. In the meantime, a solution of APS (ammonium persulfate) will be prepared and degassed. Mold assembly will be performed with two glass plates sealed by silicone tape of 1 mm thickness; a nylon sheet will be placed between the glass plates before sealing. After degassing, 5 μL of APS will be added to the monomer mixture. 1 mL of this mixture will be transferred to a 2 mL tube, and 0.5 μL of TEMED (tetramethylethylenediamine) will be added. Immediately after, the mixture will be transferred with a syringe into the glass plate for free-radical polymerization and placed into an oxygen-free nitrogen chamber. Polymerization will continue for 1 hr. Next, MiliQ water will be pipetted into the hydrogel to rehydrate and de-mold it. DGP will be washed with several water changes to eliminate unreacted monomers and initiators. DGP will be preserved in a sealed bag in MiliQ water for further use at 4° C.

### Preparation of Dyed DGP

Dyes can be incorporated by two mechanisms: adsorption and chemical reaction. Adsorption dyes will be incorporated, incubating the DGP into a 3% W/V dye in MiliQ water overnight in the shaker. The excess dye will be removed through washes with MiliQ water. Chemically attached dyes, such as reactive dyes, will be incorporated into DGP, preparing a 3% W/V dye bath at pH 5 adjusted with acetic acid. DGP will be incorporated into the bath and left overnight in the shaker at room temperature. The excess dye will be removed with MiliQ water washes until the solution is clear.

Quality control for DGP will be performed through FT-IR, comparing the spectrum of free monomers against the spectrum of the final product to ensure that the C═C signal is not present, suggesting successful polymerization.

### User Acceptability Survey Human Participants

A standardized survey was conducted to investigate user satisfaction between 2018 and 2019. 210 participants were interviewed in this study. The survey included questions regarding user acceptability and ways of integrating the device into the country's healthcare system. The questions were qualitative and quantitative in nature. The survey was conducted, and the assessment was performed based on their experience in TB diagnosis, treatment and associated stigma. The participants were asked question based on quality of the cup and their use. The participants gave their feedback on the prototype of the device ergonomics, instruction clarity, and preference over using sputum as a diagnostic sample. Data were visualized using bar graphs.

### Human Participates Who Donated Urine

Urine samples (N=24) were collected from consented hospitalized patients in Peru. Active pulmonary TB was confirmed microbiologically using auramine staining for acid-fast bacilli in sputum and (Microscopic Observation Drug Susceptibility (MODS) assay. Auramine staining was scored on a scale of 0 to 3, with 3 being the highest. The study was conducted under informed consent; with the approval of review board of the Universidad Peruana Cayetano Heredia (Lima, Peru) and Johns Hopkins Bloomberg School of Public Health (Baltimore, MD). Clinical and demographic data included sex, age, previous TB diagnosis, appetite, weight, self-reported symptoms such as fever, cough, fatigue, and hemoptysis, with average number of coughs in the previous 24 hours. The urine samples were centrifuged at 3 rcf for 10 min, and the supernatants were stored in liquid nitrogen or −80° C. until use. Samples were analyzed using urinary dipstick testing (Multistix GP, Siemens) for cystitis, proteinuria, hematuria, and specific gravity analysis for each case. Urinary nitrites were also scored but not used as exclusion parameters. Control urine samples (N=8) were collected from healthy donors in the USA. The samples were analyzed using a rapid urine analysis strip from Siemens and centrifuged at 2.0 rcf for 10 min at room temperature to remove cellular debris. The supernatant was transferred to a new tube and stored at −80° C. for future use.

### Fabrication of Collapsible Cup

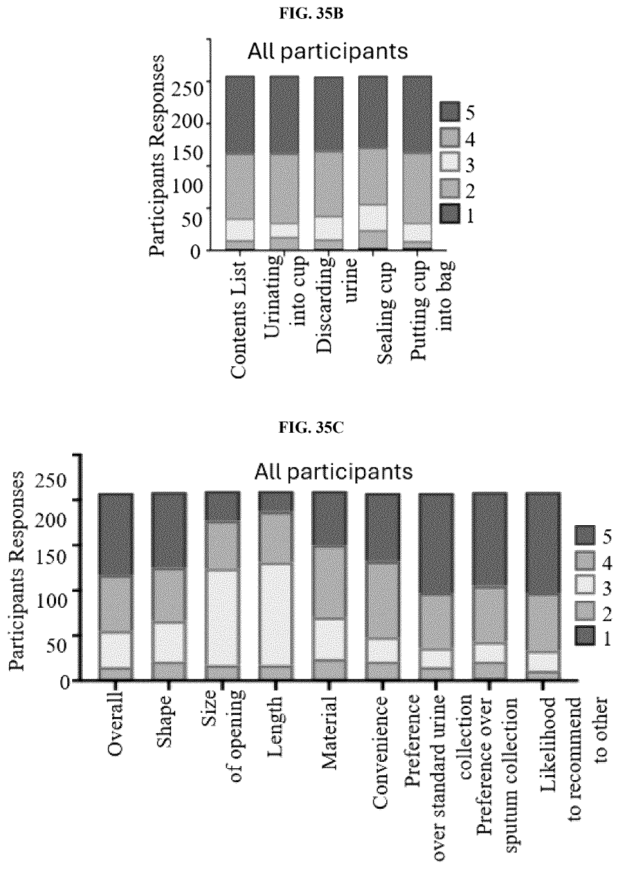

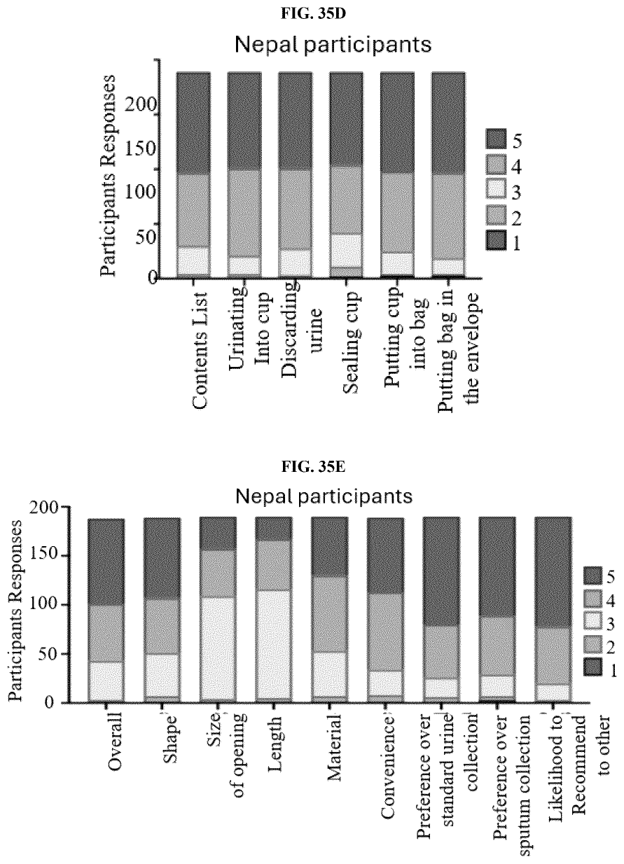

Commercial-grade polypropylene sheets were cut using an electronic cutting machine (Cricut Explore 3) using the template (FIGS. 35A-35E), folded and glued to achieve the required shape.

### Affinity Net Preparation: Dyed Nylon

Nylon sheets (20 mg, Greenlakes Fiber) were washed with 50 mL of 0.1% Alconox solution in DI water for 15-20 minutes and then with 50 mL of detergent-free MilliQ water. A dye bath was prepared by titrating hydrochloric acid to a pH range of 4-5.5. Aliquots of 0.5 g and 0.15 g of dye (Sudan Black B) and 0.02 g of dispersing agent (Alconox) were dissolved in 5 mL of dye bath. Nylon was added and incubated at 130° C. for 1 h in an IR Lab Dyeing Machine TD130 (ATI Corporation of North America). At the end of the incubation period, the mixture was allowed to reach room temperature. Washing was conducted with wash solution 1 (0.1% Alconox in water) for 15 min six times, with 100 ml of wash solution 1 each time. The nylon was then washed with wash solution 2 (0.02% Triton X-100). The nylon was washed in 100 ml of wash solution 2 for 15 min under rotation. The nylon was allowed to dry in a convection oven at 40C overnight.

### Affinity Net Preparation: Nylon-Hydrogel Composite

A 10% (w/v) monomer solution was prepared by dissolving 100 mg N-isopropylacrylamide (NIPA, Thermo Fisher Scientific, cat #412781000), 5 mg N, N′-methylenebisacrylamide, (BIS, Sigma-Aldrich, cat #M7279), and 10 μL of Allylamine (AA, Aldrich, cat #241075-50 ML) in 2 mL of deionized water. The monomer mixture was degassed under nitrogen flow for one hour. A 2 mL solution of 10% ammonium persulfate (APS Sigma-Aldrich, Cat. No. A3678) was prepared and degassed separately. After degassing, the APS solution was added to the monomer mixture and mixed thoroughly followed by 10 μL of tetramethylethylenediamine (TEMED, Bio Rad, Cat. No. 1610801). A sheet of Sudan black B dyed nylon was placed between two glass plates, clipped, and sealed. The dimensions of the glass mold were 10 cm×10 cm×0.01 cm. The monomer solution was immediately transferred into the glass mold using a syringe for free-radical polymerization and placed in an oxygen-free nitrogen chamber overnight. The next day, MilliQ water was pipetted into the composite to rehydrate and facilitate demolding. The nylon needs to undergo several washes with MiliQ to remove residual unreacted monomers and initiators.

### uEV Extraction Using Ultracentrifugation

Urine samples (˜15 mL) from patients were thawed at room temperature and centrifuged at 5 rcf for 10 min at room temperature to remove debris. The supernatant from the spin was collected in 26.3 mL polycarbonate tubes and supplemented with 1X phosphate-buffered saline (PBS without calcium and magnesium) to reach a 22.5 mL volume. The samples were centrifuged at 100 rcf for 1.5 h at 4° C. in a Beckman coulter optima xe-100 ultracentrifuge. The supernatant was discarded, and the pellet was washed with PBS at 100 rcf for 1.5 h at 4° C. The supernatant was discarded, and the pellet was collected by adding 200 μL of PBS and stored at 40C.

### uEV Extraction Using the Affinity Net

A 0.25 cm² nylon-hydrogel composite was incubated with 50 μL urine for 10, 15, and 30 min. The sheet was then transferred to a new tube and eluted with an elution buffer containing 2M urea, 3.85% SDS, and water. The elution buffer was collected and used for downstream processing of the samples.

### SDS PAGE and Immunoblot Analysis

The ultracentrifugation samples 10 ul were resuspended in 10 ul of Laemmli buffer (Biorad, cat #1610737), heated for 5 minutes at 95° C., and loaded onto Novex Tris-Glycine 4-20% Gel (ThermoFisher). Proteins were separated using sodium dodecyl sulfate-polyacrylamide gel electrophoresis (120 V for 1.5 hours) and stained using the Pierce™ Silver Stain Kit (ThermoFisher, cat #24612) according to manufacturer's instructions. Silver-stained images were generated using an Azure biosystem c300. For western blotting, proteins were transferred to a polyvinylidene difluoride (PVDF) membrane at 25 mV for 2 h. For dot blot, samples were manually spotted on a nitrocellulose membrane (0.4 μm, BioRad). The membrane was blocked in 1X-PBST with 5% milk for 45 min at room temperature and incubated with primary antibody CD63 (abeam ab315108) at 1:1000 overnight at 4C and washed with 1X-PBST. The membrane was then treated with a secondary antibody (anti-rabbit) at 1:10000 for 1 h at room temperature. The cells were washed with 1X-PBST. The membrane was developed using SuperSignal™ West Dura Extended Duration Substrate (Themofisher, cat #34076). The image was generated using an Azure biosystem c300.

### Mass Spectrometry Analysis

The EV samples were mixed with 20 μL of 8 M urea and reduced with 10 mM dithiothreitol at 50° C. for 5 min. The mixture was alkylated with 50 mM iodoacetamide at room temperature for 15 min and digested with trypsin at 37° C. for 4 h. The samples were desalted using ZipTip (Millipore, Ref #ZTC18S096), dried in a SpeedVac, and reconstituted with 10 μL of 0.1% formic acid for mass spectrometry (MS) analysis. Liquid chromatography-tandem mass spectrometry (LC-MS/MS) experiments were performed using an Exploris 480 (ThermoFisher Scientific, Waltham, MA, USA) equipped with a nanospray EASY-nLC 1200 HPLC system. Peptides were separated using a reversed-phase PepMap RSLC 75 m i.d.×15 cm long with a 2 μm particle size C₁₈ LC column from ThermoFisher Scientific. The mobile phase consisted of 0.1% aqueous formic acid (mobile phase A) and 0.1% formic acid in 80% acetonitrile (mobile phase B). After sample injection, the peptides were eluted using a linear gradient from 5% to 40% B for over 90 min and then ramped to 100% B for an additional 2 min. The flow rate was set to 300 nL/min. The Exploris 480 was operated in a data-dependent mode in which one full MS scan (60,000 resolving power) from 300 m/z to 1500 m/z was followed by MS/MS scans in which the most abundant molecular ions were dynamically selected and fragmented by higher-energy collisional dissociation (HCD) using a collision energy of 27%. “EASY-Internal Calibration,” “Peptide Monoisotopic Precursor Selection,” and “Dynamic Exclusion” (15 s duration) were enabled, as was the charge state dependency, so that only peptide precursors with charge states from +2 to +4 were selected and fragmented. Tandem mass spectra were searched against the NCBI human database using Proteome Discover v 2.3 from ThermoFisher Scientific. The SEQUEST node parameters were set to use full tryptic cleavage constraints with dynamic oxidation of methionine. The mass tolerance for precursor ions was 2 ppm, and that for fragment ions was 0.02 Da. A 1% false discovery rate (FDR) was used as a cutoff value for reporting peptide spectrum matches (PSM) from the database search.

### PCR Analysis

The PCR reaction of 20 μL were performed with 5 ng of urinary EV DNA and 10 μL of Syber Green Master Mix (Biorad Cat #1725270), 0.1 μM of rpoB forward primer and 0.1 μM of rpoB reverse primer. The samples were run on a QuantStudio 7 Pro (Applied Biosystems) at a melting temperature of 72° C. separated on a 1% agarose gel with 6X SYBR™ Safe DNA Gel Stain (Thermo Fisher, cat #S33102) in 1X-Tris-Borate-EDTA (TBE) running buffer at 100V for 80 minutes. The images were generated using an Azure biosystem c300.

### Bioinformatic Analysis

Human uEV-associated proteins were identified using mass spectrometry. An Interactive Venn Diagram tool was used to identify the common proteins. Human organ specificity was determined using databases such as GeneCards, Bgee, and the Tissue database (Jensenlab). Organ specificity was established using a location confidence ranging from to 4-5 and an expression score of more than 95%.

### Example 1: Lyme Disease Diagnosis Preliminary Data

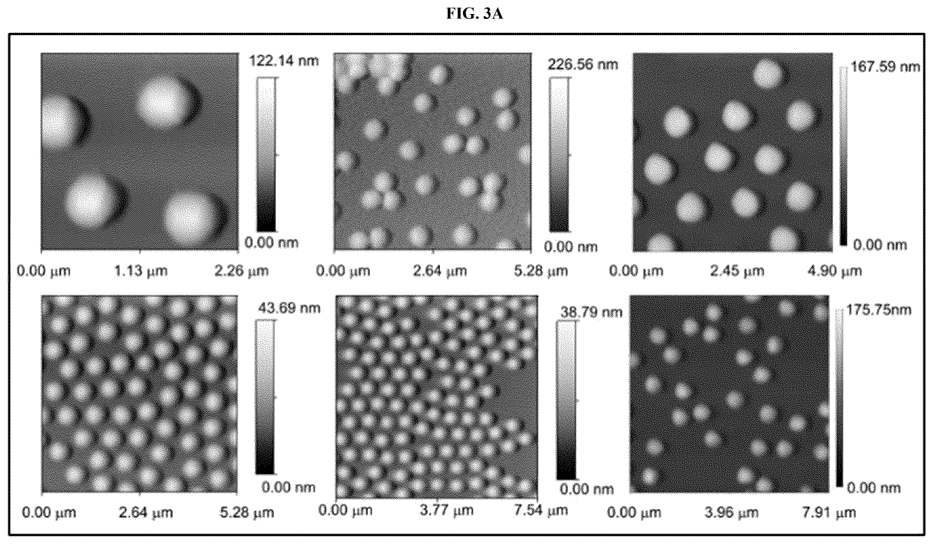

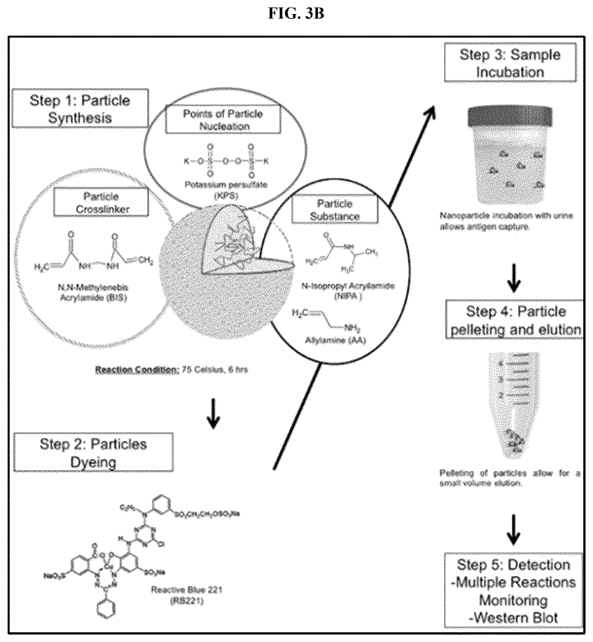

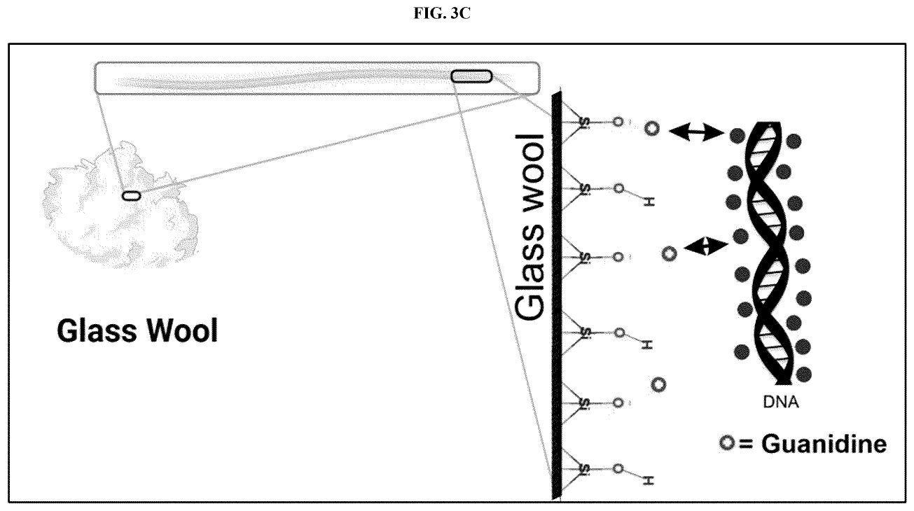

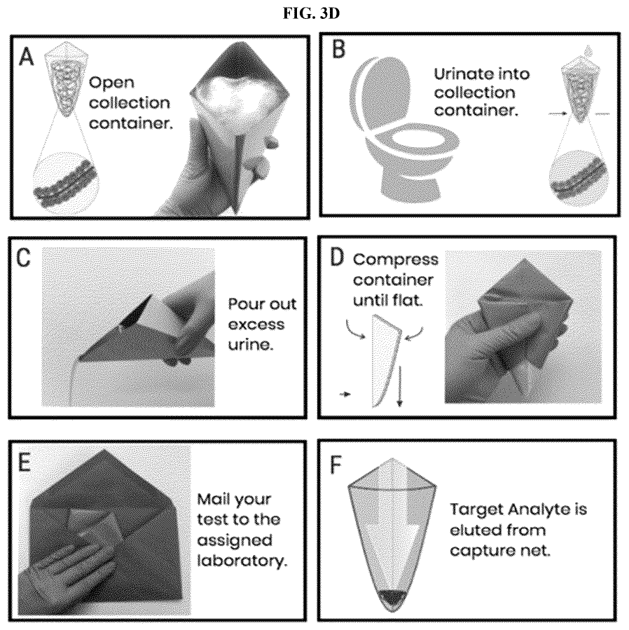

As shown in FIGS. 3A-3D, a range of solid-based affinity-binding technologies have been designed and utilized to tailor for specific applications. Core-shell nanoparticles such as NIPA/AAc (acrylic acid) and NIPAm/C.B. (Cibacron Blue) (FIG. 3A) showed a successful enrichment of Toxoplasmosis urine-derived antigens, enhancing their detectability via mass spectrometry analysis (FIG. 3B). This is further exemplified using glass wool affinity fibers to capture urine-derived DNA (FIG. 3C), and the Urigami cup, a patient-friendly collection device integrated with glass wool to streamline the process of analyte collection for further proteomics analysis (FIG. 3D).

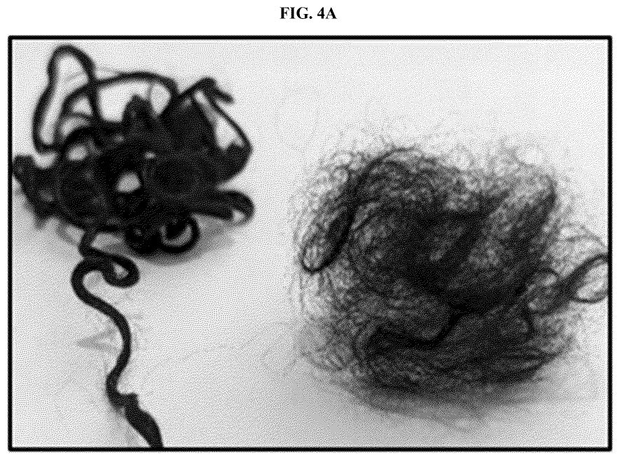

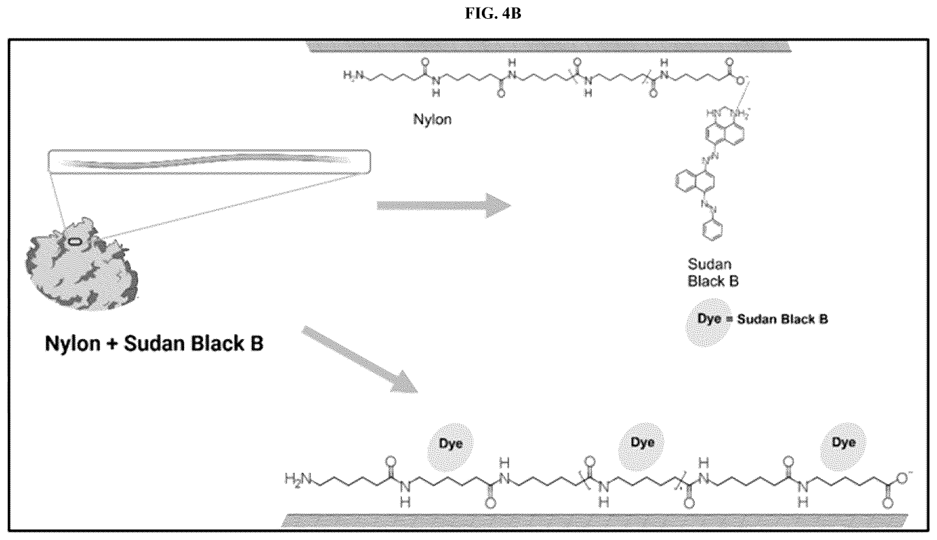

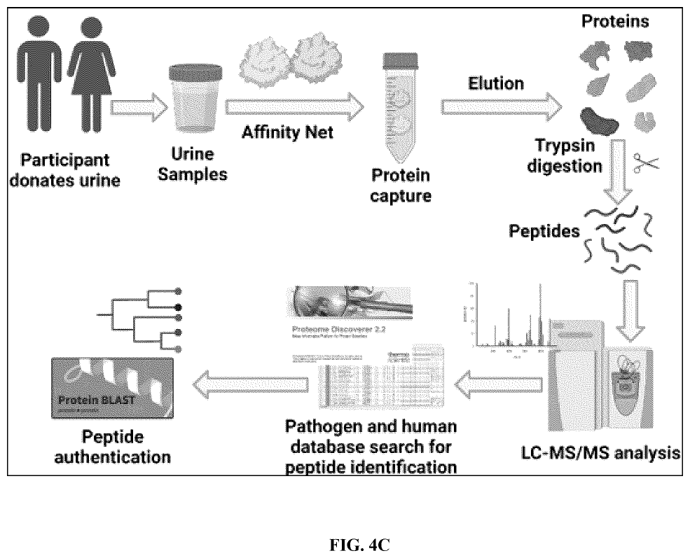

Nylon dyed with Sudan Black B exemplifies the molecular interactions driving the dye-nylon affinity process (FIGS. 4A-4B). The schematic in FIG. 4C provides an insight into this innovative approach for recovering Borrelia-derived biomarkers and EVs from patient urine, followed by mass spectrometry analysis. Gel electrophoresis (FIGS. 2E-2F) provides compelling evidence of this technology's efficacy in capturing all the urine-derived biomarkers (Input 1-3) and releasing them into a smaller, more concentrated solution. Protein enhancement can be observed by comparing lanes with urine subjected to enrichment (Elution 1-3) against those without (Input 1-3). This validates the assay and emphasizes its potential advantages in enhancing the sensitivity and specificity of Lyme-derived biomarker detection. Examples of Lyme-derived peptides identified using mass spectrometry are shown in Table 1.

TABLE 1
Example Borrelia-specific peptides identified
in the urine of affected patients

| Peptide Sequence | Protein annotation |
|---|---|
| YFDNLSEEDVLKLN | Crystal structure of a |
| IPTGIPLVYELDK | phosphoglycerate mutase |
| (SEQ ID NO: 1) | gpmA |
| LGEVVAFFEYLNSV | Ribosome maturation |
| FLEVR | factor RimM |
| (SEQ ID NO: 2) | |
| LENTAMPLVAEIGE | Flagellar motor switch |
| VK | protein FliM DNA |
| (SEQ ID NO: 3) | mismatch repair |
| | endonuclease |
| INTYDIPQNNNLET | MutL |
| EDVNEPNK | |
| (SEQ ID NO: 4) | |
| IVDIDESNPNLPYI | HD domain-containing |
| NYIIK | protein |
| (SEQ ID NO: 5) | |
| NYLNEPSRDIITK | Hypothetical protein |
| (SEQ ID NO: 6) | |
| SLEVLGNEYTK | Oligoendopeptidase F |
| (SEQ ID NO: 7) | |
| ISAALSDTGVTYSR | 50S ribosomal protein |
| FIEGLLK | L20 |
| (SEQ ID NO: 8) | |

Peptides were recovered from the patient's urine using a nylon-based affinity net dyed with Sudan Black B and Sudan IV.

In another aspect, the disclosure relates to hydrogel NIPA-BIS/AA (DGP); the FT-IR spectrum provides critical insights into the final structure of this material (FIG. 5A). The spectrum highlights the C═O association with other chemical groups in the complex polymer, as shown by a stretching at 1636 cm⁻¹. Further, this material was dyed with pararosaniline base dye, yielding promising results (FIGS. 5B-5C).

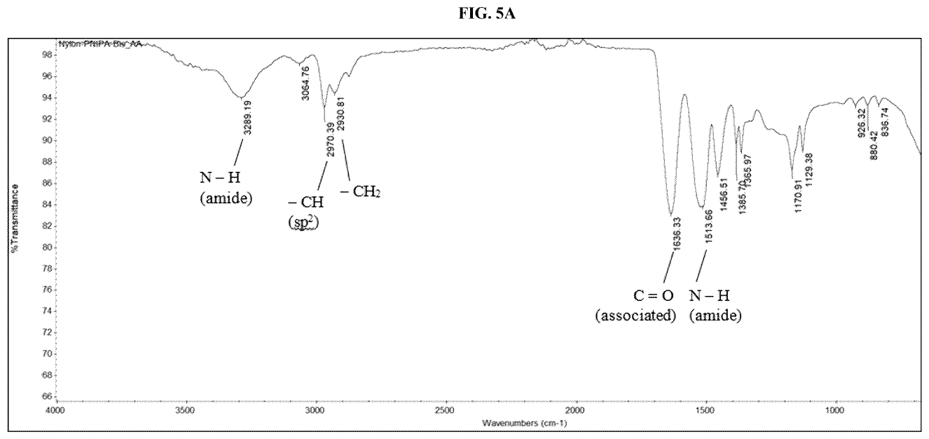

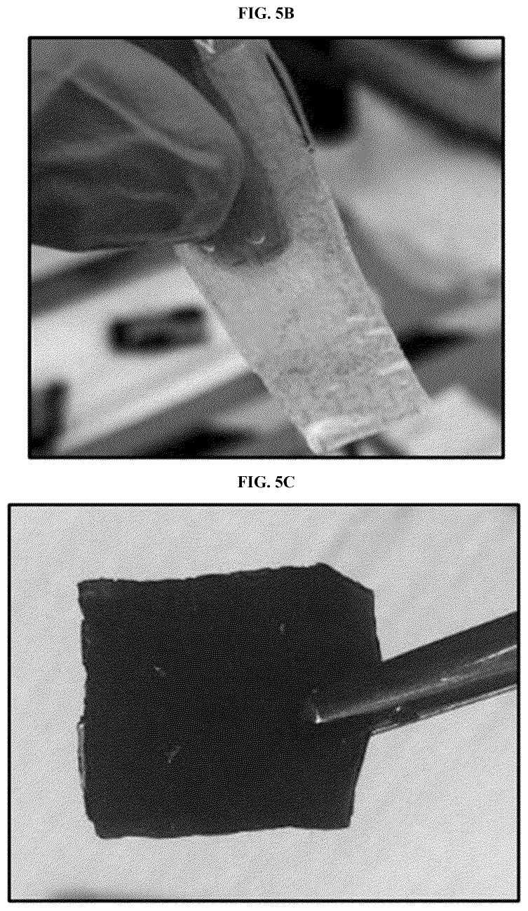

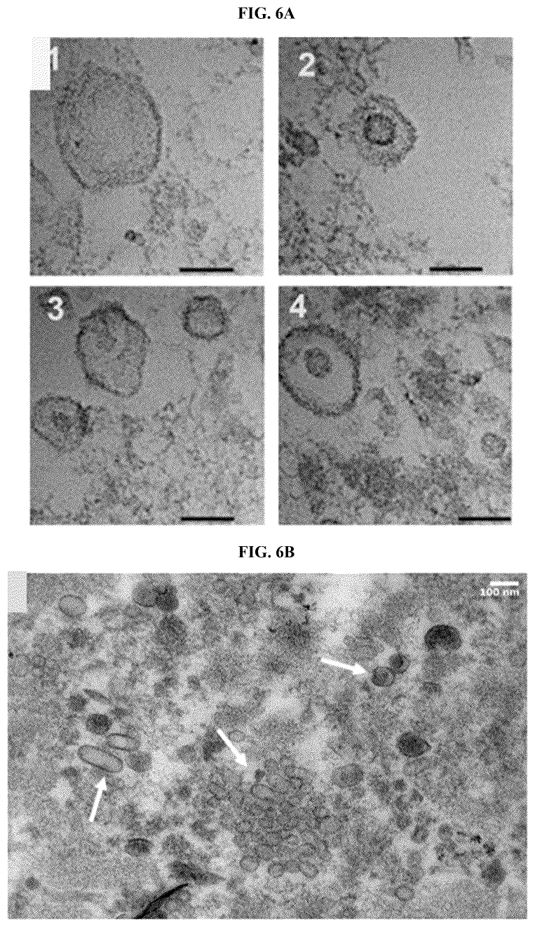

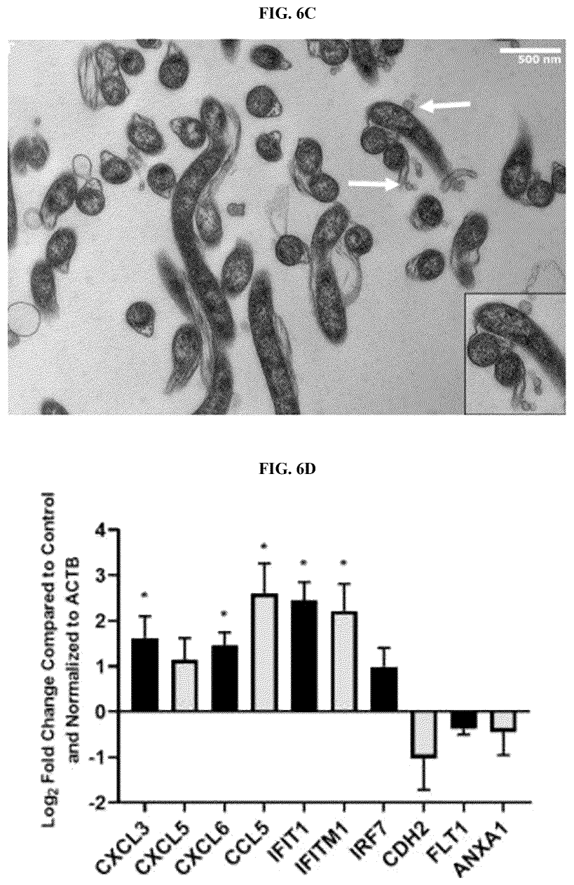

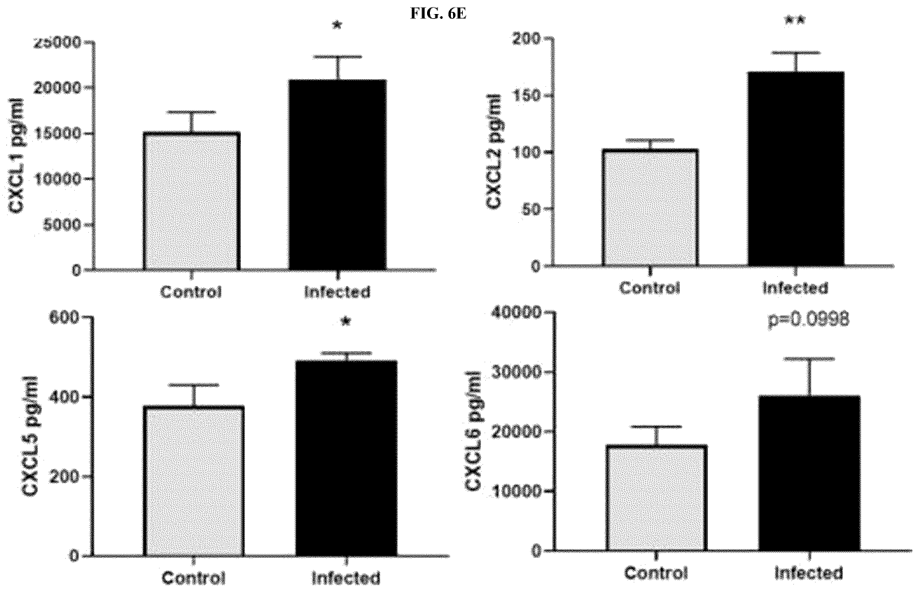

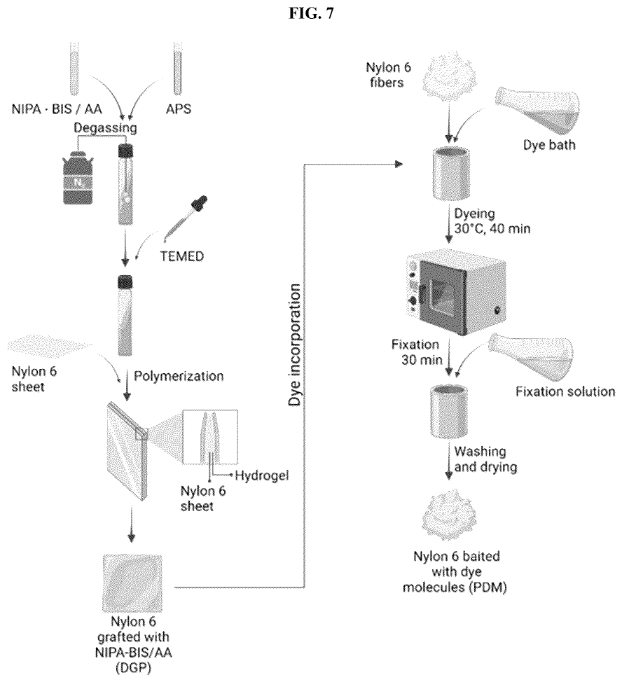

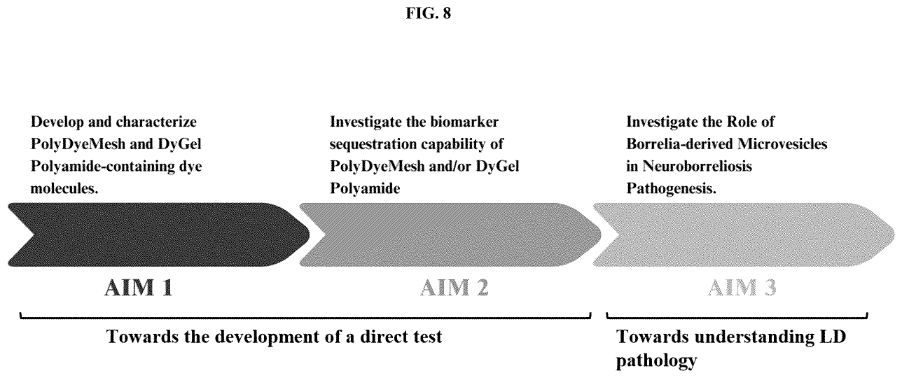

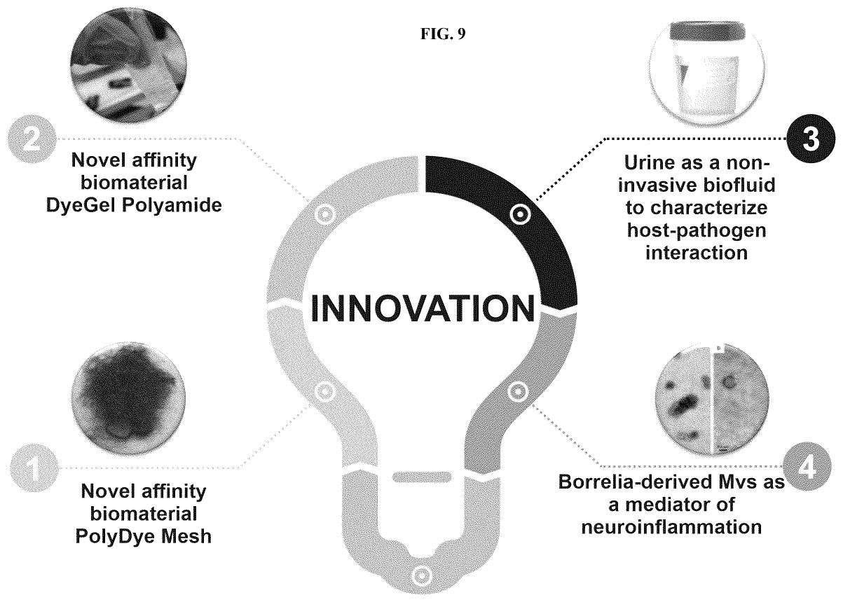

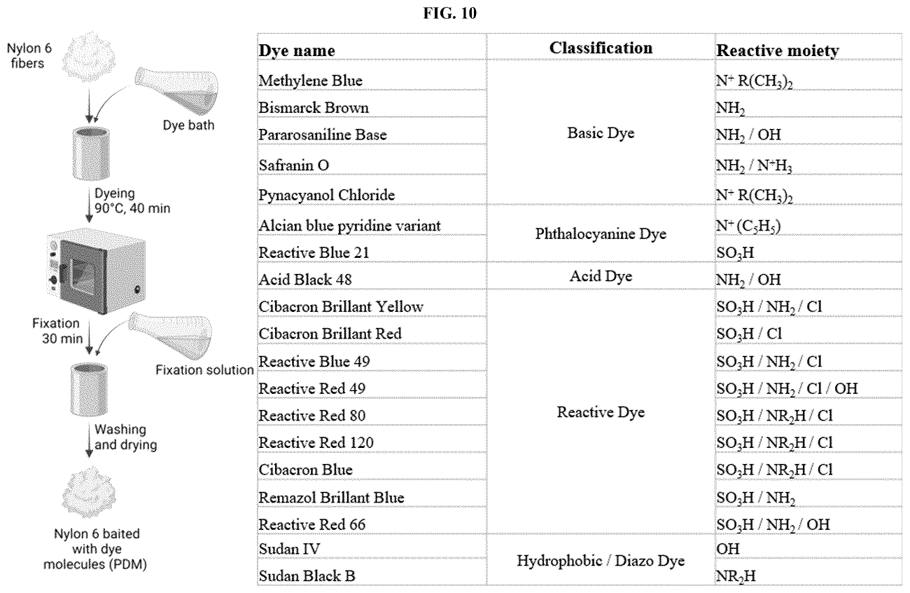

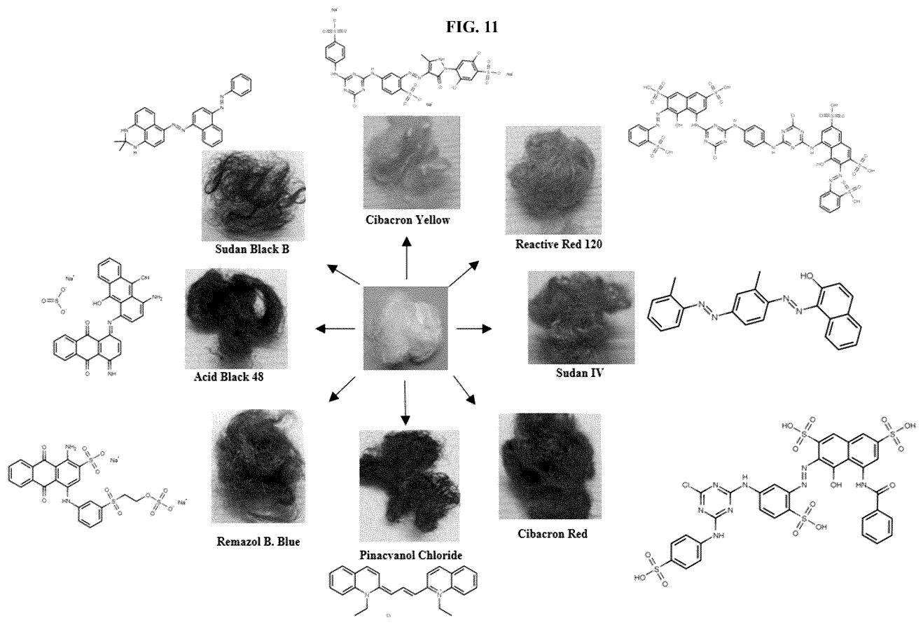

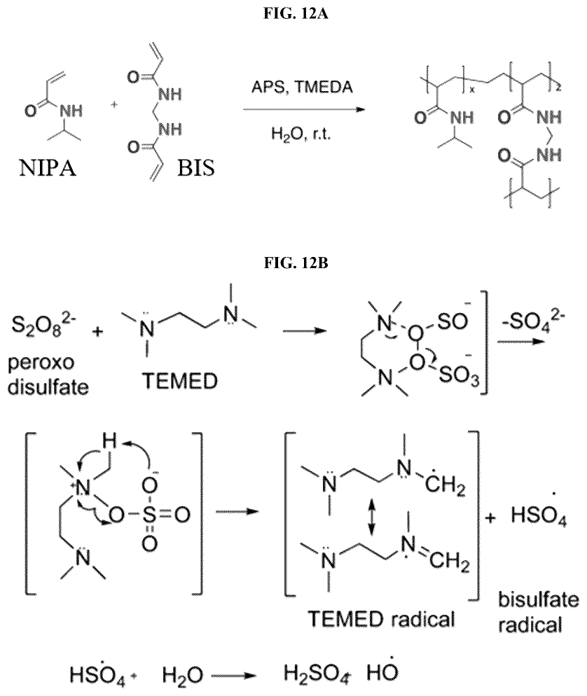

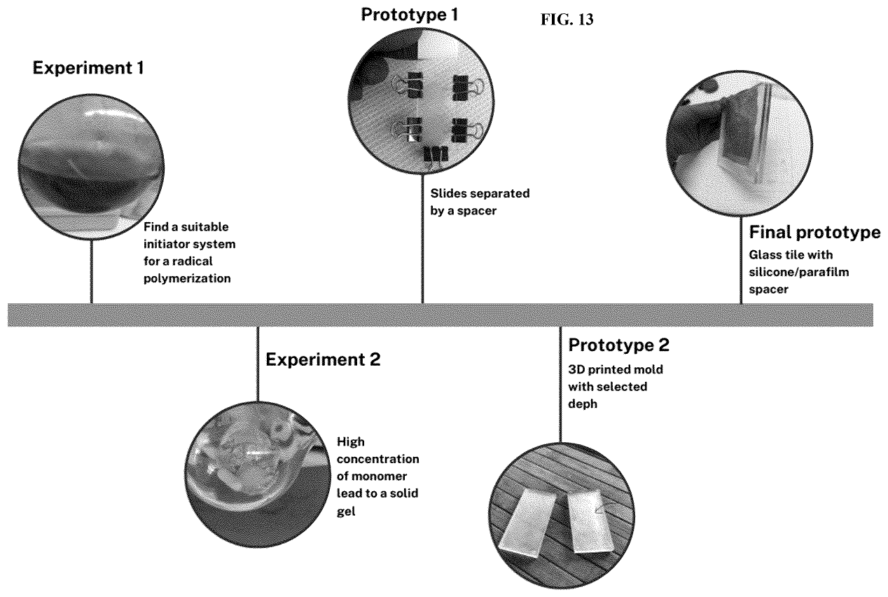

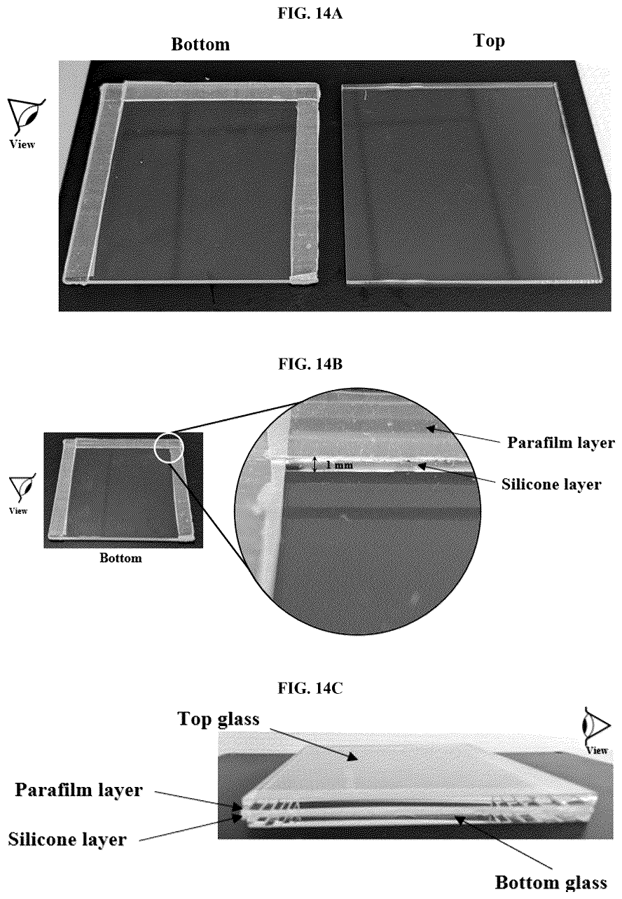

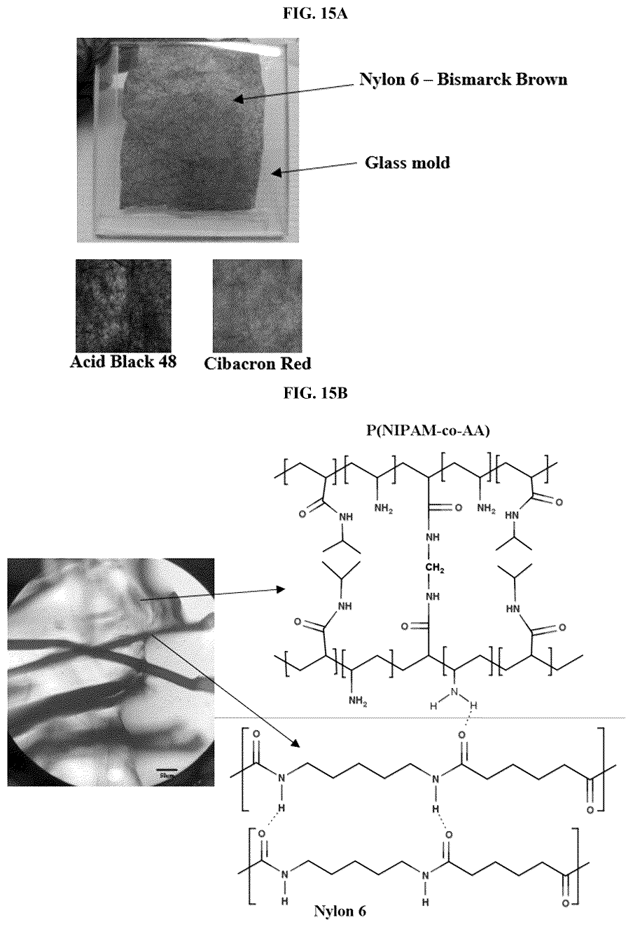

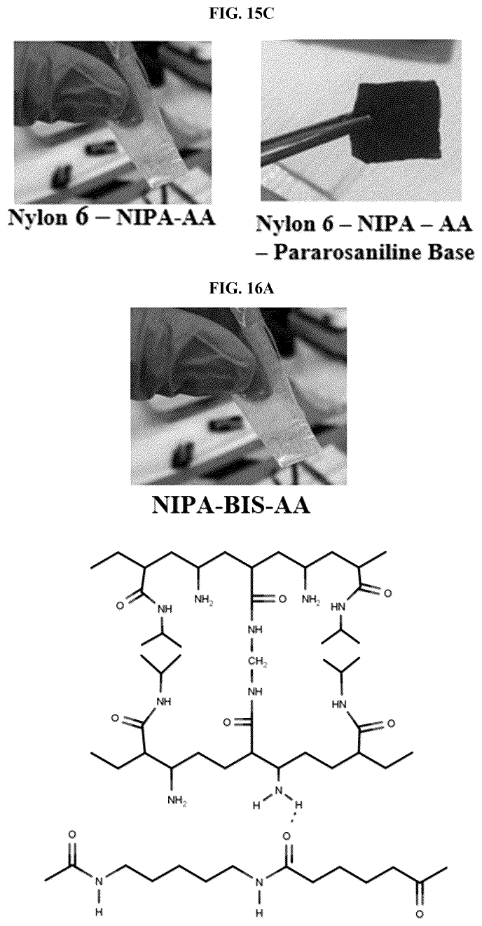

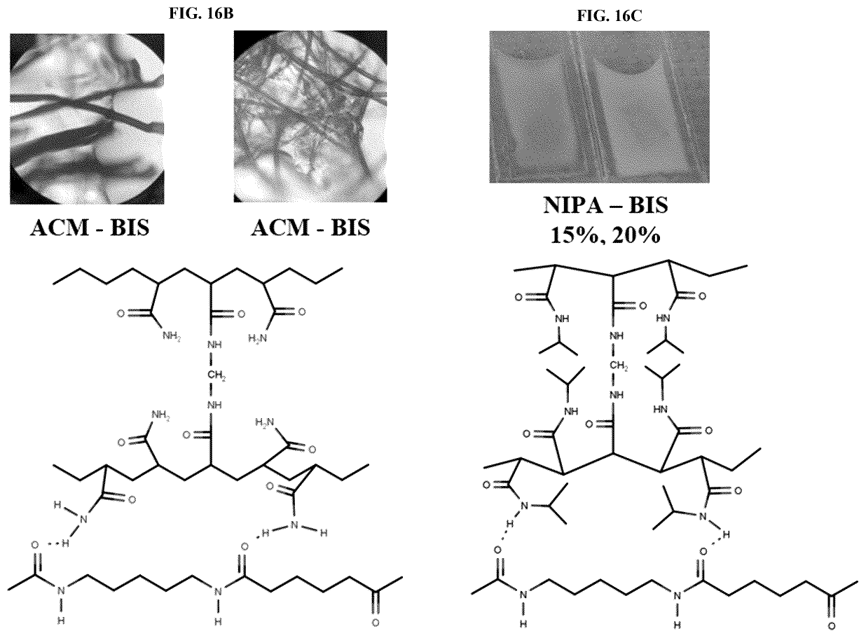

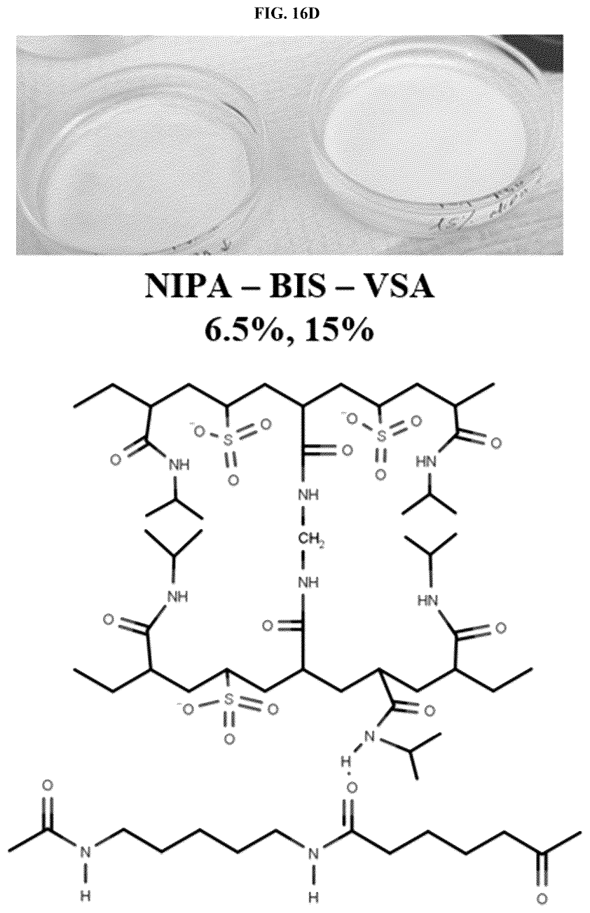

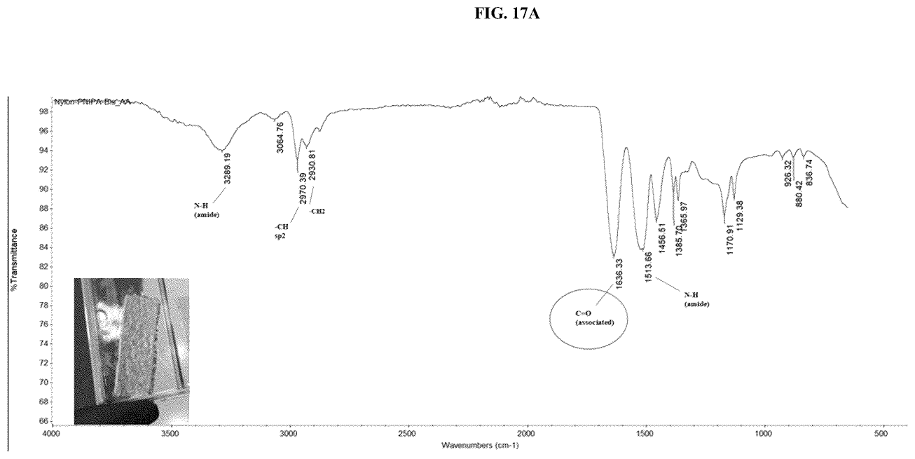

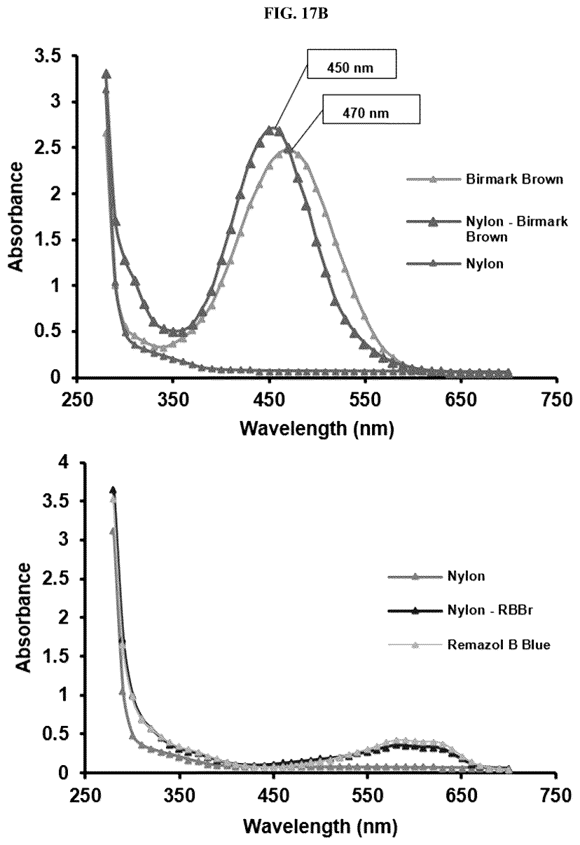

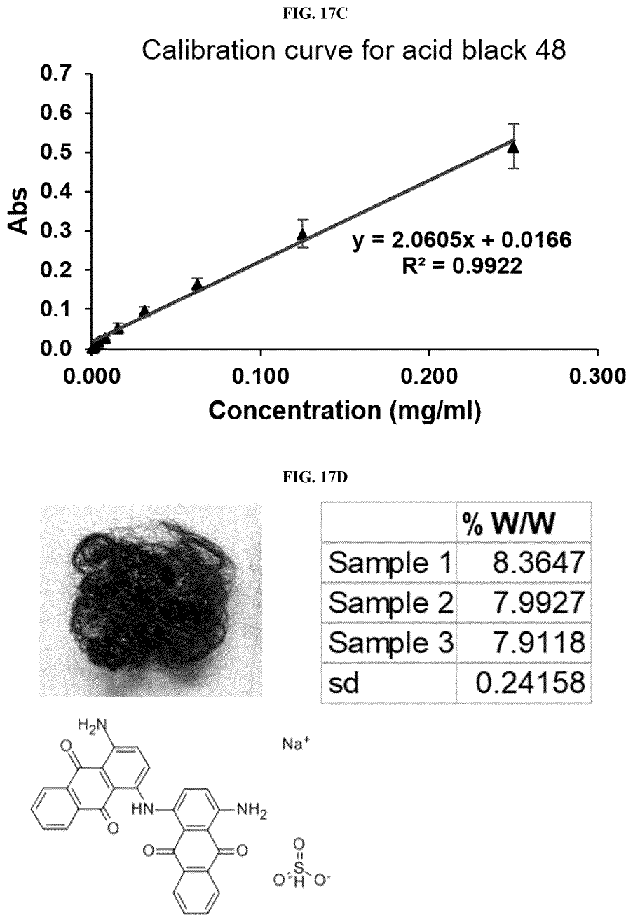

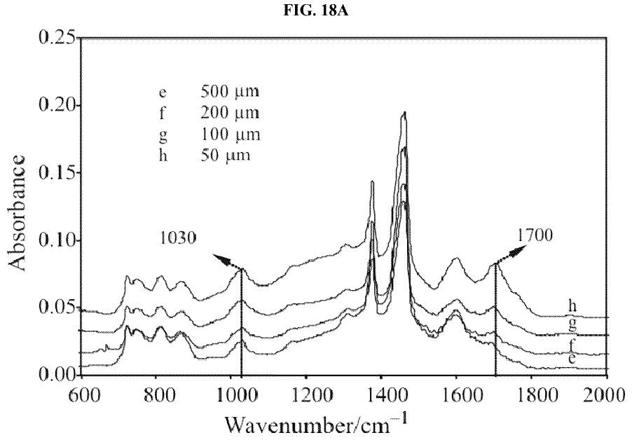

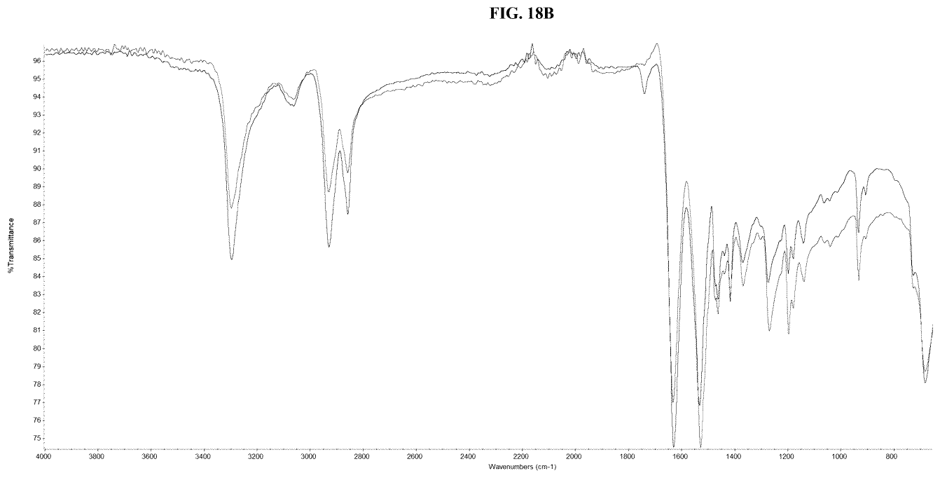

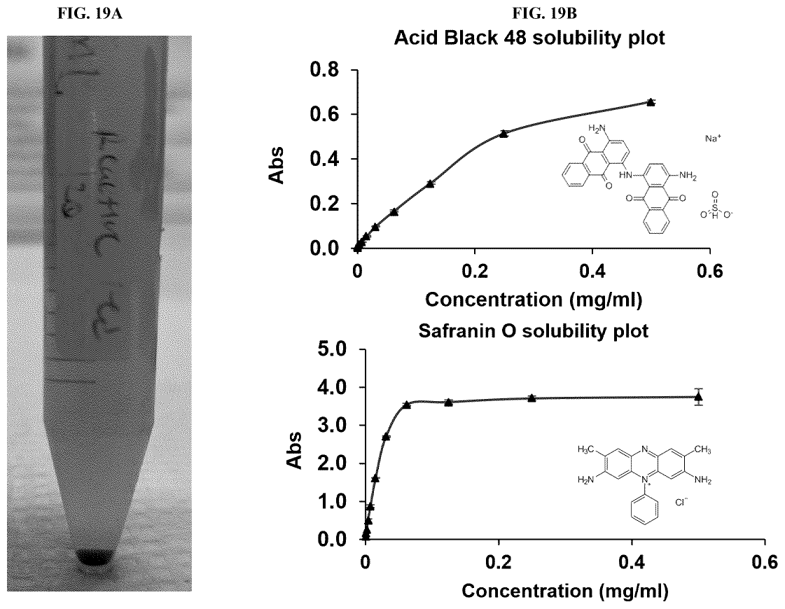

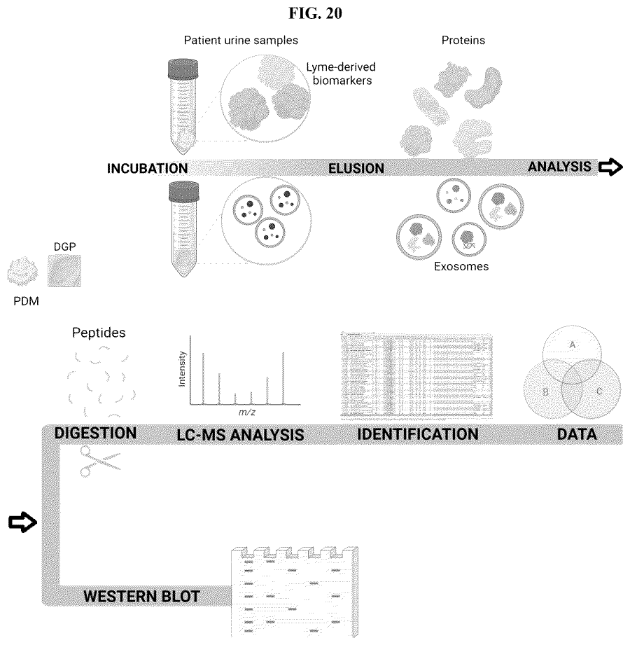

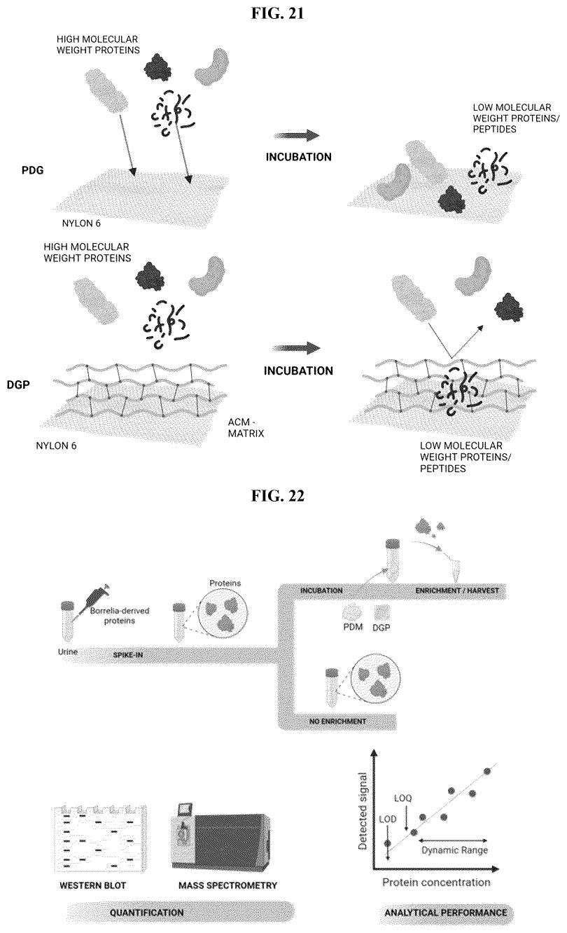

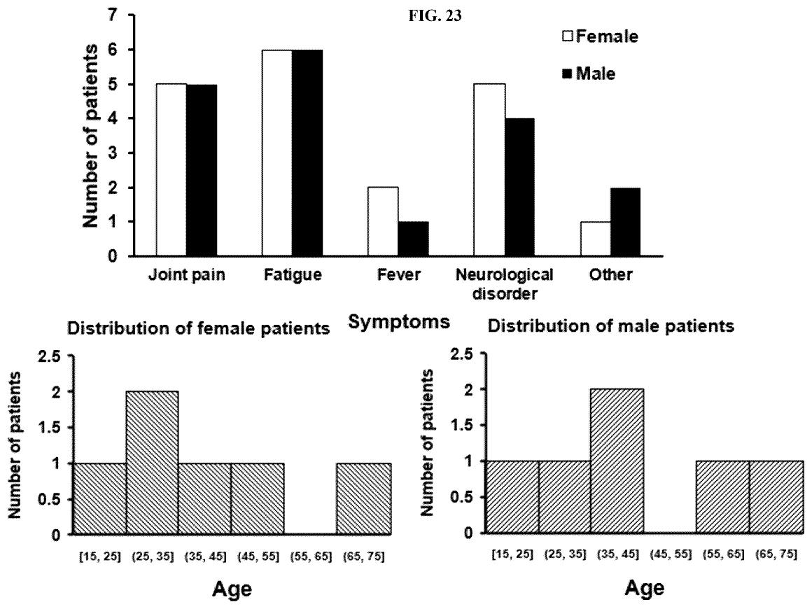

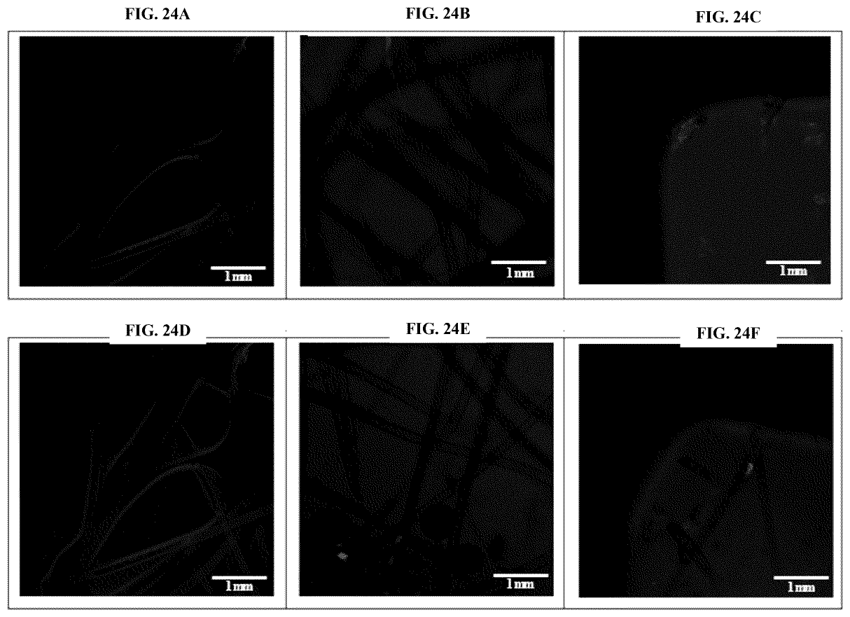

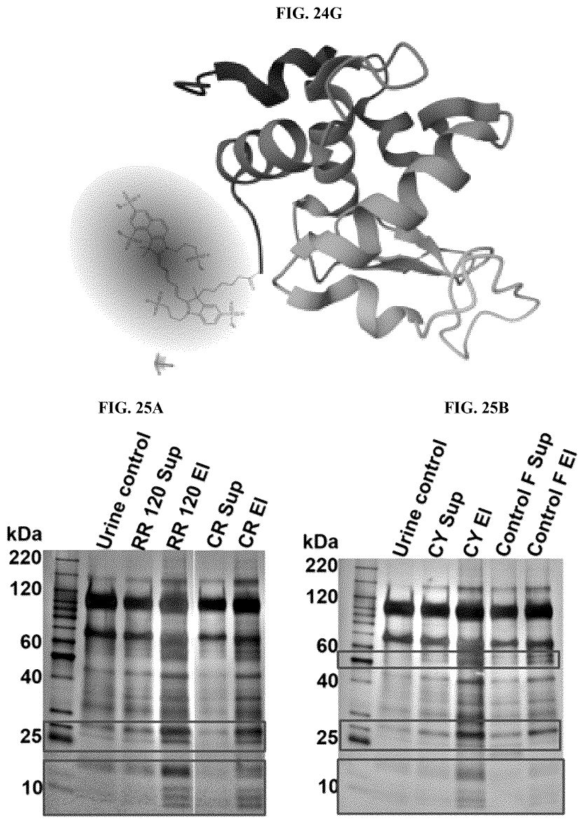

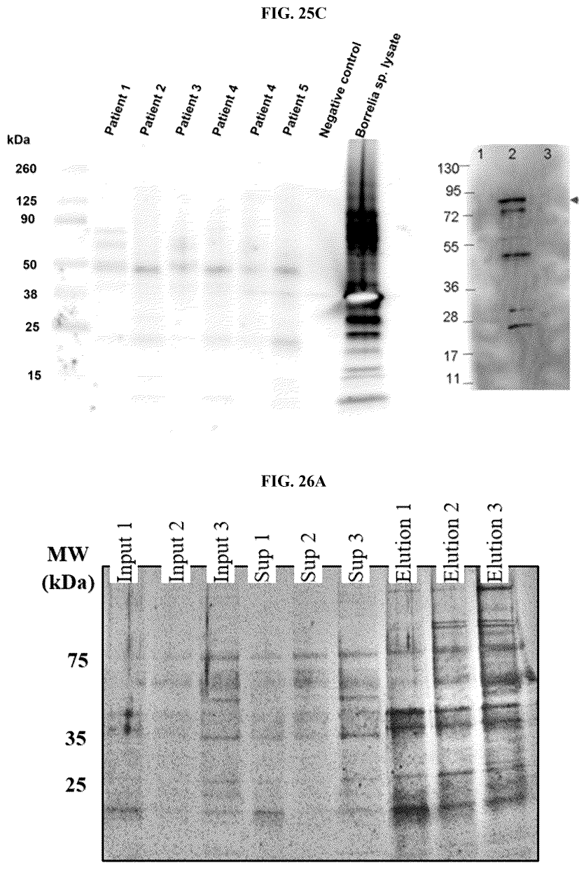

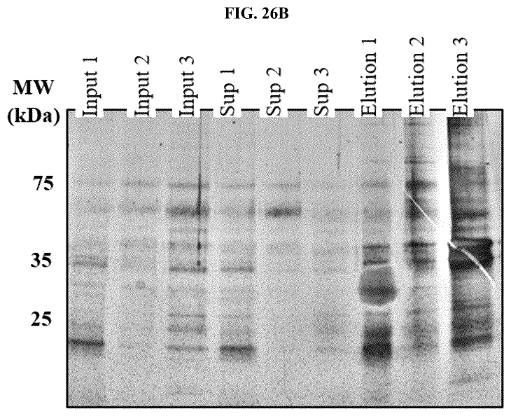

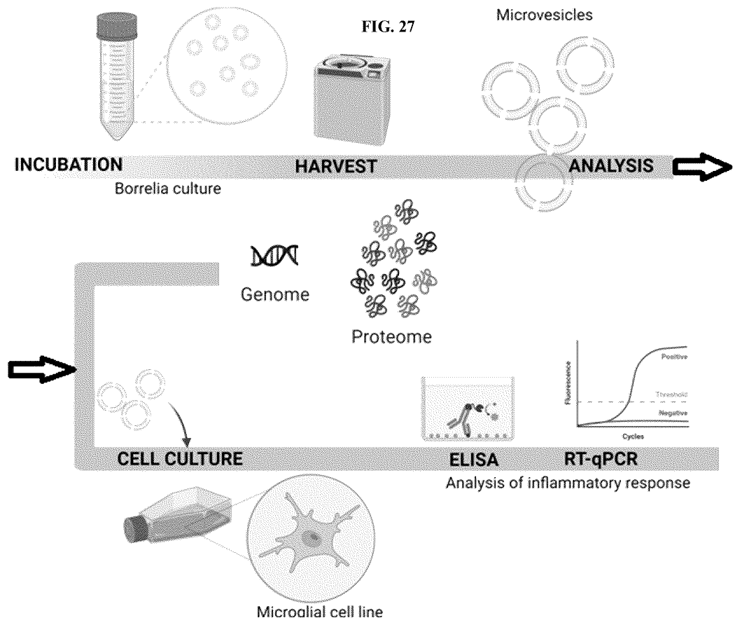

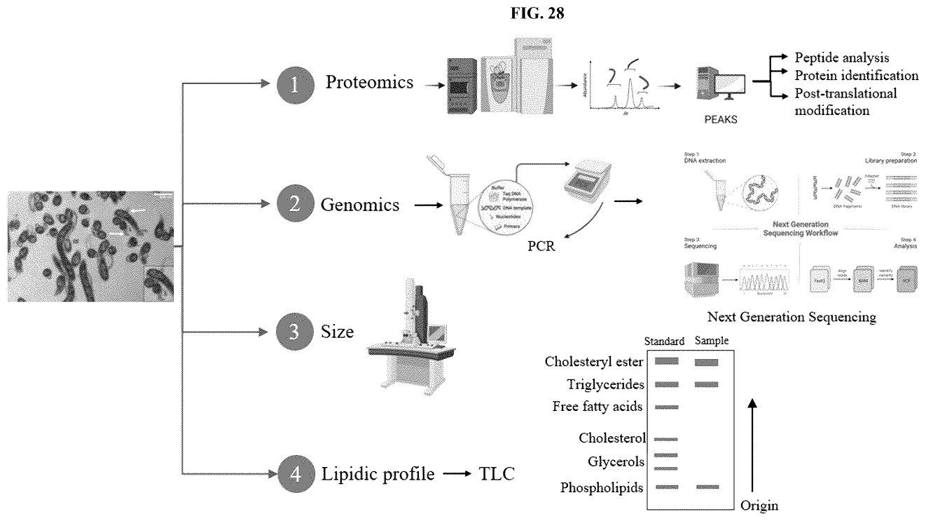

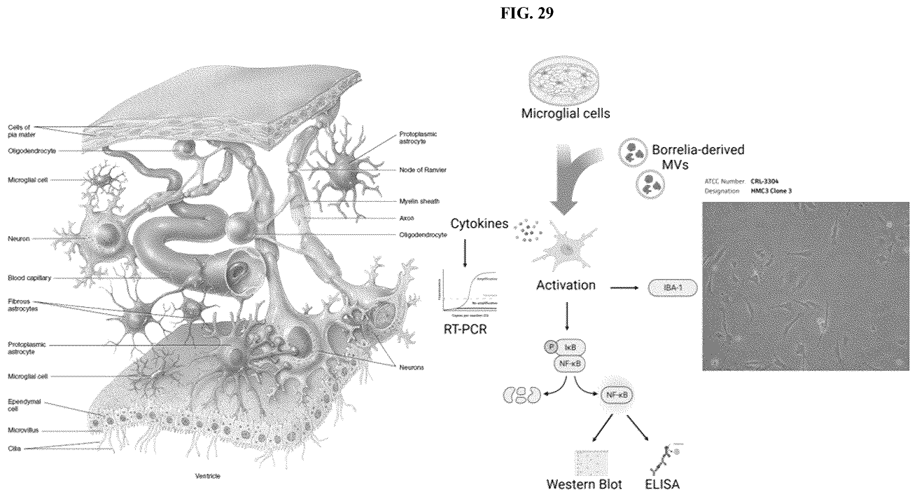

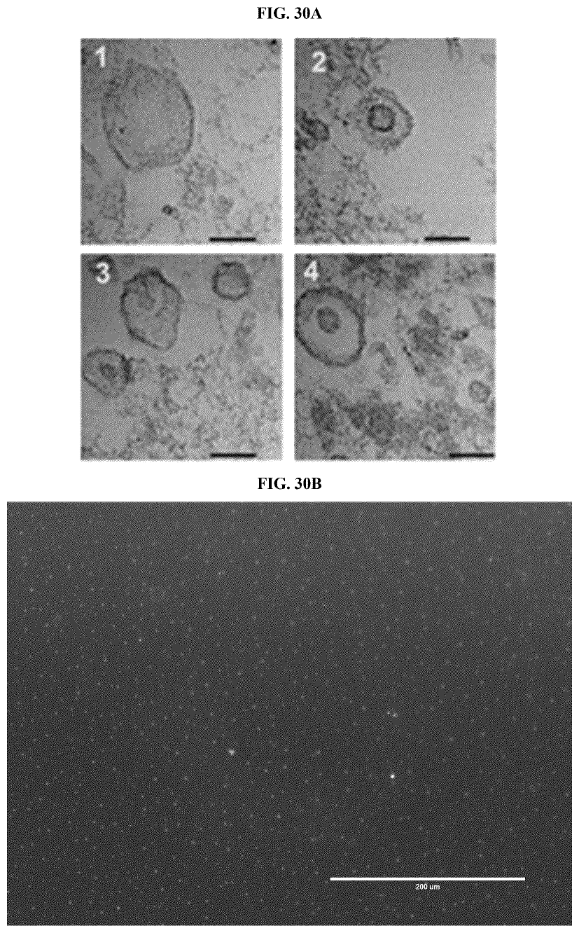

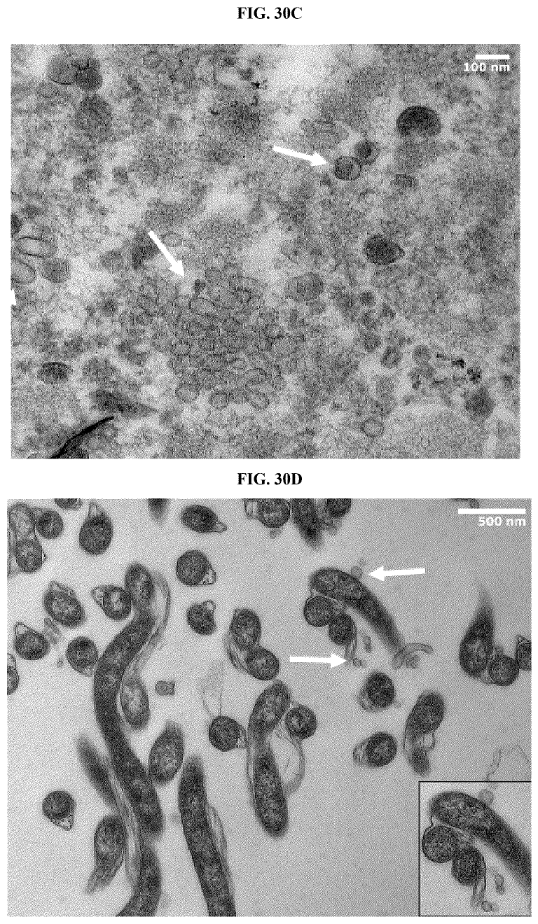

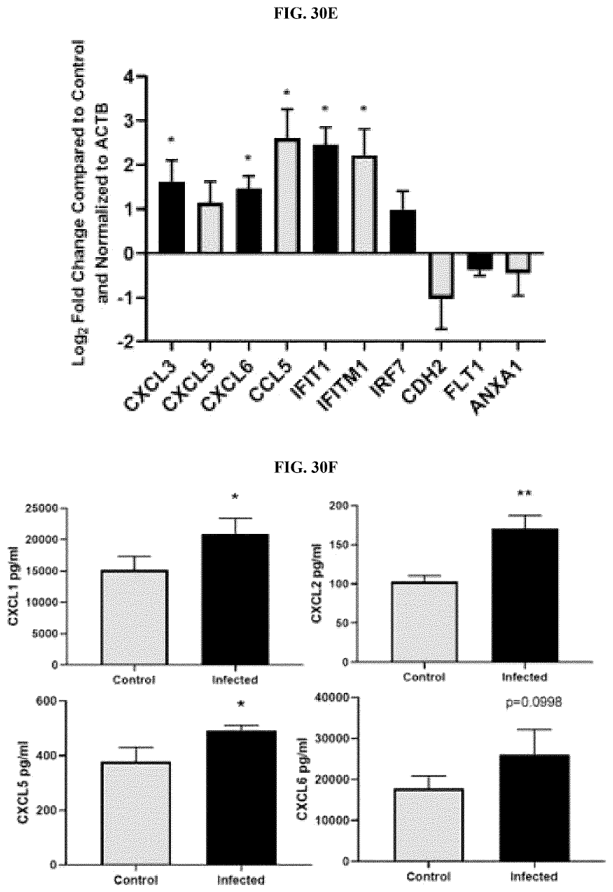

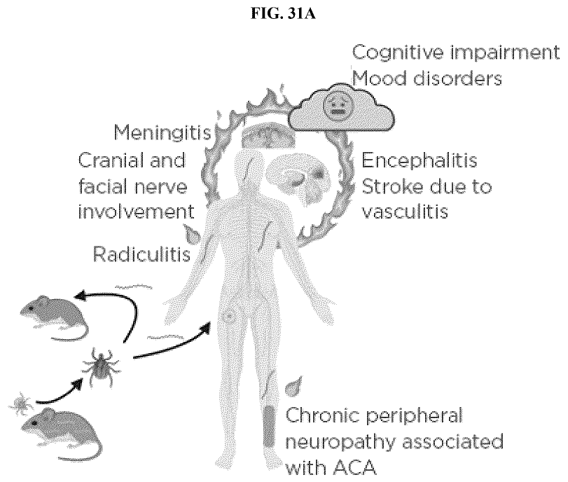

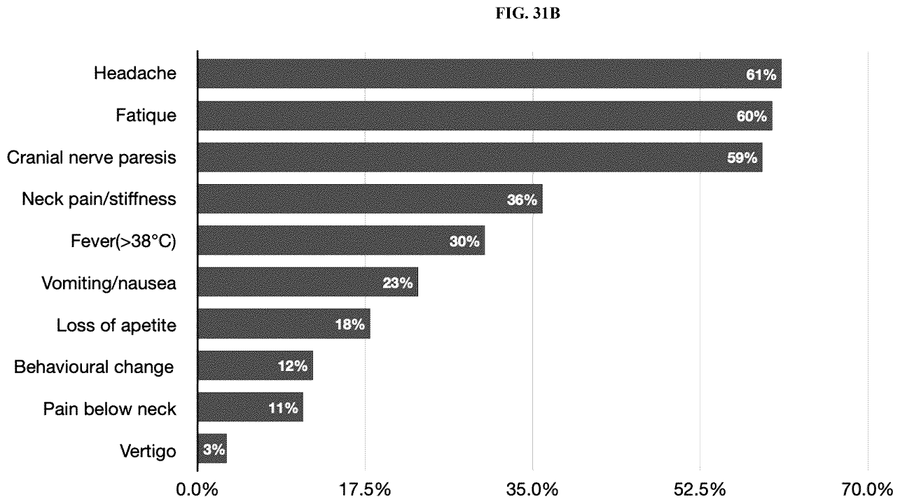

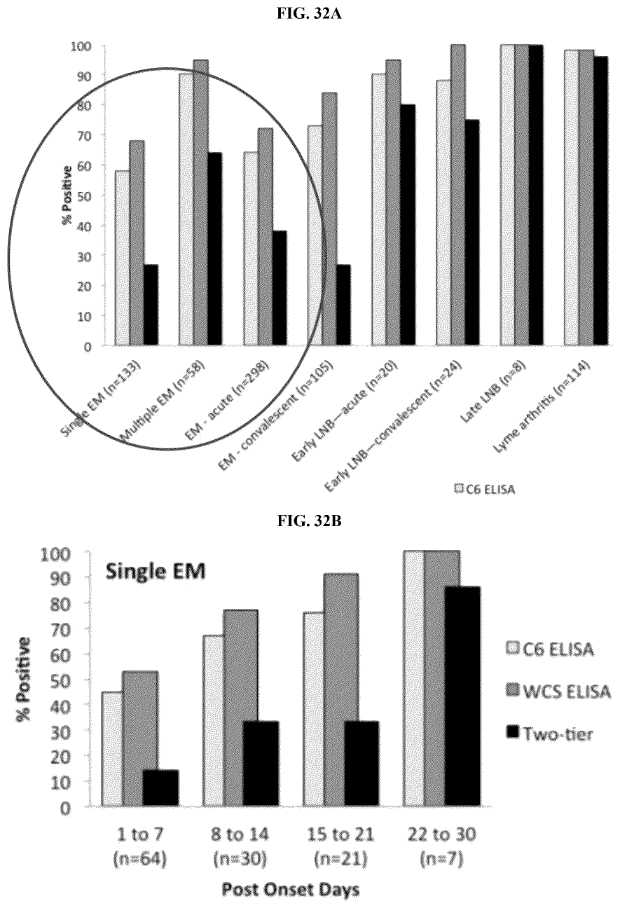

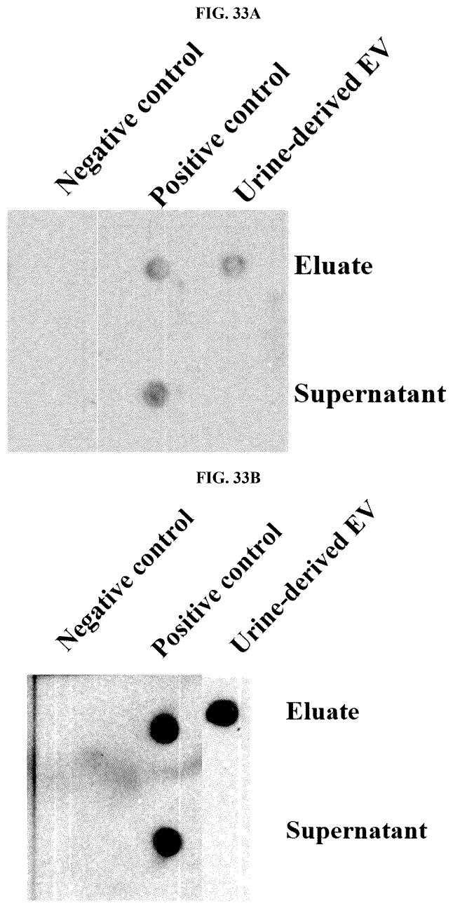

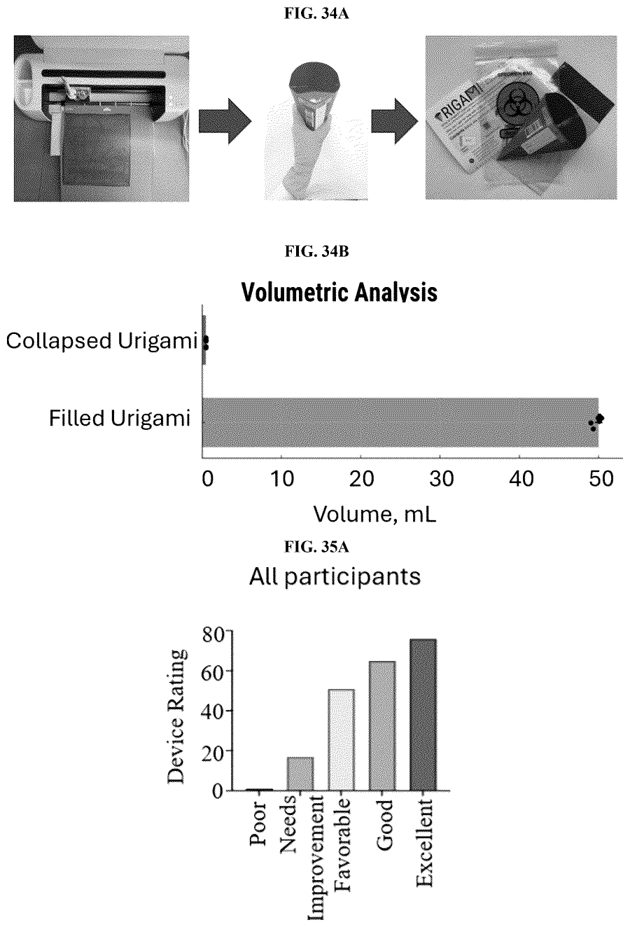

Thus, preliminary findings have successfully demonstrated the feasibility of isolating MVs from other bacterial species (FIG. 6A). Moreover, the electron microscope images (FIGS. 6B-6C) show the feasibility of isolating and characterizing Borrelia burgdorferi MVs. Further, a magnified view provides an insightful visualization of MVs budding directly from Borrelia burgdorferi (FIG. 6C). Published cell line studies show that co-culture of Borrelia with mammalian cells induces a pro-inflammatory response. FIG. 6D shows the results of a study highlighting the inflammatory response in human choroid plexus epithelial cells post Borrelia sp. exposure. In addition to the interactions above, another critical component of this preliminary evidence lies in the cytokine profiles observed (FIG. 6E), painting a portrait of the cellular response triggered by Borrelia sp. Such data accentuates the importance and relevance of this research direction. Building upon these foundational insights, it has been hypothesized that the interaction of Borrelia sp. with neuronal cells may result in distinct inflammatory pathways.

TABLE 2
Exemplary hydrogel composition formulations

| | | Reactive | | |
|---|---|---|---|---|
| Monomer 1 | Monomer 2 | Group | % T | % C |
| NIPA | | | 20%, 15% | 3%-5%-12% |
| NIPA | AA | NH₂ | | |
| NIPA | AAc | COOH | | |
| NIPA | VSA | S(═O)₂O⁻ | 6.3%, 15% | |
| ACM | | | 12.5%, 20% | 2.7%-3.3%-5% |

BIS crosslinker used; NIPA (n-isopropylacrylamide); N,N-bisacrylamide; AA (allylamine); AAc (acrylic acid); VSA (vinylsulfonic acid); and ACM (acrylamide).

### Example 2: a Collapsible Urine Collection Device for Capturing Urinary Bioanalytes

In one aspect, the disclosure describes the development of a collapsible urine collection device containing an affinity net, which captures and concentrates urinary bioanalytes for downstream analysis. The device, referred to as “Urigami,” integrates the affinity net into a conical cup fabricated from non-imbibing, waterproof materials such as vinyl (FIG. 3D). The geometric design maximizes fluid handling capacity (up to 50 mL) while allowing easy compression into a flat format for mailing.

The device is intended for point-of-need use with minimal user burden. As illustrated in FIG. 3D, the user opens the container, urinates directly into the device, and allows sufficient incubation time for the affinity net to capture target analytes. Excess urine is then poured out, and the device is compressed into a two-dimensional form. Once sealed in a mailing envelope, the sample can be shipped at ambient temperature to a diagnostic facility. Upon receipt, analytes are eluted from the affinity net for laboratory testing.

Volumetric analysis demonstrates that the device achieves approximately 500-fold concentration, reducing a 50 mL urine sample to <0.1 mL of retained material following compression. This innovation can support safe, low-cost, and infrastructure-independent transport of urine-derived molecular targets, facilitating decentralized diagnostic workflows.

### Example 3: High User Satisfaction with the Urigami Collection Cup

To assess usability and user satisfaction, a prototype urine collection kit was deployed and structured surveys were administered to a total of N=210 participants, including 190 individuals in Nepal and 20 in Guinea-Bissau. Survey responses were based on a 5-point Likert scale, with 1=least satisfied and 5=highly satisfied (see Supplementary Materials for full questionnaire).

Across all participants, 91.4% reported overall satisfaction, defined as a rating of 3 or higher, and 67.1% of users rated the device with scores of 4 or 5, indicating strong approval (FIG. 35A). Satisfaction was consistently high across different aspects of the urine collection process, including kit contents, ease of urination, urine disposal, sealing, and packaging. As shown in FIG. 35B, over 90% of users rated these components favorably (>3). Device characteristics were also evaluated for design and usability. As illustrated in FIG. 35C, participants expressed high satisfaction with the usability of the Urigami cup was 92% of overall satisfaction (>3). 89.5% of participants preferred urine collection over sputum collection, suggesting broad acceptance of urine as a non-invasive alternative for TB diagnostics (FIG. 35C). Despite overall positive feedback, certain usability features received mixed responses. As shown in FIG. 35C, approximately 50-54% of participants rated the cup's length and opening size as acceptable, while recommending increasing both dimensions for easier handling. Region-specific data revealed especially high satisfaction in Nepal, where 96.7% of participants rated the cup >3, and 71% provided ratings of 4 or 5 (FIG. 35E). Nepali participants favored the packaging and mailing process, with 88.4% reporting that inserting the Urigami into the transport bag and envelope was convenient and well-conceived (FIG. 35D). More than 99% of Nepali participant reported they would highly likely recommend the device to a friend (FIG. 35E).

Taken together, these results demonstrate high user acceptability and point to minor areas for design optimization, supporting further scale-up of the Urigami device for decentralized TB diagnostics.

### Example 4: Urine Sample Donors

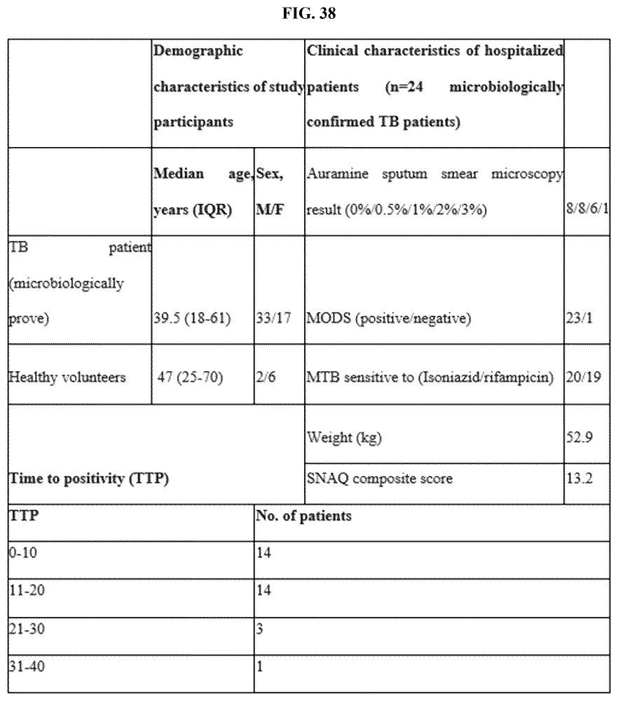

In order to validate the affinity net capability to capture and sequester the uEVs, banked urine samples from 24 hospitalized patients with tuberculosis in Lima, Peru and healthy controls in the United States were studied. The median age of the participants was 39.5. FIG. 38 shows the clinical characteristics of the patient participants. The median age ranged from 18 to 61 years for TB (+/) participants (FIG. 38) and 25 to 78 years for healthy controls. Most of the patients were male (66%). The TB positivity was confirmed through sputum smear microscopy tests. The Microscopic Observation Drug Susceptibility (MODS) study showed that 23 patients were positive, and 1 patient were negative. The patients visited the clinic with self-reported symptoms of cough, hemoptysis, fever, fatigue, HIV, and diabetes. It also showed the patient visited clinic with TB symptoms and suffering from HIV and diabetes, as these factors play an important role in TB diagnosis. The study also included urine collected from 8 healthy patients in the United States, with median age of 47 and 2 males. From all these participants urine was rapidly frozen upon collection and stored at −80° C. until further use.

### Example 5: Urinary EVs Contain Proteins Deriving from Multiple Organ Systems Including the Lungs

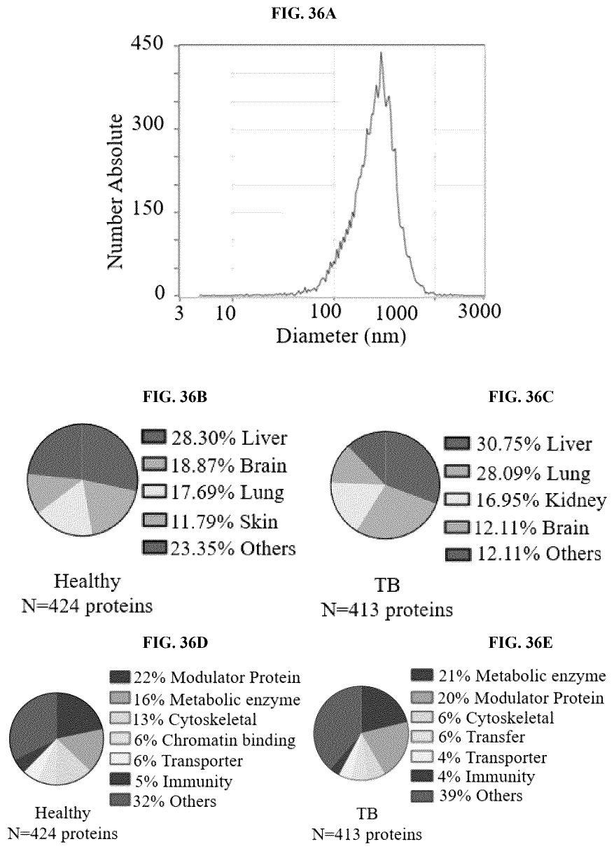

Urinary extracellular vesicles (uEVs) were isolated according to standard procedures and characterized using morphological and molecular analysis. Nanoparticle size distribution showed a uEV quantity of 10⁸ to 10⁹ EVs per mL, and a vesicle SD (+/−) 137.75 nm (FIGS. 36A-36B), consistent with large extracellular vesicles and exosome aggregates. FIG. 36A, shows a median diameter of 259.9 nm, with a size distribution ranging from 125.9 to 422.4 nm (X10-X90). The mean size was 278.2 nm and the standard deviation was 133.8 nm. The sample concentration after dilution was 6.3×10⁷ whereas the original concentration was 6.3×10⁹ particles/mL. A total of 11,195 particles were tracked and around 378 particles were found in each frame.

Tandem mass spectrometry proteomic analysis of uEVs from TB-positive patients (NP24, FIG. 38) identified canonical eukaryotic extracellular vesicle markers including CD9, CD63, and CD81, confirming the presence of tetraspannins in the urine isolate. In addition, the uEV proteome contained macrophage- and monocyte-associated proteins such as CD14 (NP_000582.1), CD9b (NP_002201 1 XP_006712574.1), MHC class II molecules (NP_001346122.1 XP0115128641 NP_072049.2, NP_002116.2),Fizz-1 (NP065148.1), and Yml (NP_653247.1), as well as epithelial and immune markers such as cadherin 1 (NP_061749.1), HLA class I (NPm001229687.1), HLA class II (XP_024308334.1), and tetraspanin family member CD151 (NP944492.1). These markers suggest that uEVs originate from diverse immune and epithelial cell types, possibly reflecting systemic immune activation and epithelial remodeling.

TABLE 3
TB biomarker, macrophage biomarker and tetraspanin
in urine of TB positive patient

| Accession | Description |
|---|---|
| NP_001760.1 | CD9 antigen isoform 1 [Homo sapiens] |
| NP_001284578.1 | CD81 antigen isoform 2 [Homo sapiens] |
| NP_001120699.1 | CD59 glycoprotein preproprotein [Homo sapiens] |
| NP_001189486.1 | CD44 antigen isoform 8 precursor [Homo sapiens] |
| NP_001120699.1 | CD59 glycoprotein preproprotein [Homo sapiens] |
| NP_001244321.1 | CD63 antigen isoform C [Homo sapiens] |
| XP_011538762.1 | tetraspanin-1 isoform X1 [Homo sapiens] |
| NP_002406.1 | macrophage migration inhibitory factor [Homo |
| | sapiens] |
| NP_054701.1 | Macrophage lysosome-associated membrane protein-2 |
| | (LAMP2) |
| NP_001171597.1 | immunoglobulin lambda-like polypeptide 5 isoform 1 |
| | [Homo sapiens] |
| XP_011538762.1 | tetraspanin-1 isoform X1 [Homo sapiens] |
| NP_005898.2 | mannosyl-oligosaccharide 1,2-alpha-mannosidase IA |
| | [Homo sapiens] |
| NP_002108.4 | HLA class I histocompatibility antigen, Cw-1 alpha |
| | chain precursor [Homo sapiens] |
| XP_024308335.1 | HLA class II histocompatibility antigen, DRB1-4 beta |
| | chain isoform X2 [Homo sapiens] |
| NP_000582.1 | monocyte differentiation antigen CD14 precursor |
| | [Homo sapiens] |

To infer the anatomical sources of the proteins identified in uEVs, a bioinformatic analysis was conducted that matched mass spectrometry-derived proteins to publicly available curated organ-enriched expression datasets. In TB negative samples (N=8), the tissue contribution was different: 28.30%, 18.87%, 17.69%, and 11.79% from the liver, brain, lung, and skin, respectively (FIG. 36B). Lung-specific proteins constituted 28.09% of the TB uEV proteome compared to 17.69% in healthy controls, suggesting increased release of lung-derived EVs in the context of TB-related pulmonary damage. In TB-positive patients (N=24), the urinary extracellular vesicle (uEV) proteome showed tissue-specific signature contributions of 30.75% from the liver, 28.09% from the lung, 16.95% from the kidney, and 12.11% from the brain (FIG. 36C).

Functional categorization of the uEV proteome revealed enrichment in proteins associated with modulation, metabolic protein, cytoskeleton, chromatin binding protein, transporter, immunity respectively, and 32% other proteins for healthy individuals (FIG. 36D). On other hand, in case TB positive individuals, 21%, 20%, 6%, 6%, 4%, 4% metabolic protein, modulator protein, cytoskeleton, transfer, transporter, and immunity, respectively, were observed and 39% other proteins for TB positive individuals (FIG. 36E).

The other proteins include RNA metabolism, calcium binding, cell adhesion, extracellular matrix (ECM), scaffold, and transmembrane receptor protein. In case of tuberculosis, proteins play an essential role in the survival of the bacteria, whereas sustain the growth and adaptation. It has been observed that a TB infected individual has greater percentage of metabolites as compared to a healthy individual. Cytoskeleton protein is less in TB as compared to healthy as it aids phagocytosis and granuloma structure as TB disrupts immune cell regulation during infection. The gene expression regulation is done by chromatin protein with immunity protein shaping TB progressions. On other hand, the other proteins include RNA metabolism that regulate mycobacterial survival with calcium binding balancing the host pathogen signaling. Cell adhesion and ECM proteins controls the entry of bacteria, granuloma formation and remodeling of tissues. The scaffold protein helps in immune signaling, and the transmembrane receptor detects Mtb antigens.

Collectively, these findings support the hypothesis that uEVs reflect systemic pathophysiological processes, including immune activation and organ-specific damage, and may serve as a non-invasive source of biomarkers for TB disease monitoring.

### Example 6: The Affinity Net Efficiently Captures uEVs and Mtb Biomarkers

The affinity net is composed of non-woven nylon filaments functionalized with two affinity probes: (1) Sudan Black B, a hydrophobic dye targeting the uEV lipophilic membrane, and (2) a hydrogel containing poly(N-isopropylacrylamide-co-allylamine), which is hypothesized to target the negatively charged uEV surface.

To assess capture efficiency, the affinity net performance was compared to ultracentrifugation for the isolation of uEVs and the capture kinetic was evaluated. To do so, the Mtb biomarker LAM, total protein, and CD63 tetraspannin were monitored. Dot blot assays confirmed that the affinity net captures LAM at levels comparable to ultracentrifugation. In some cases, LAM recovery from the affinity net exceeded that obtained via ultracentrifugation. No LAM signal was observed in healthy controls, confirming assay specificity.

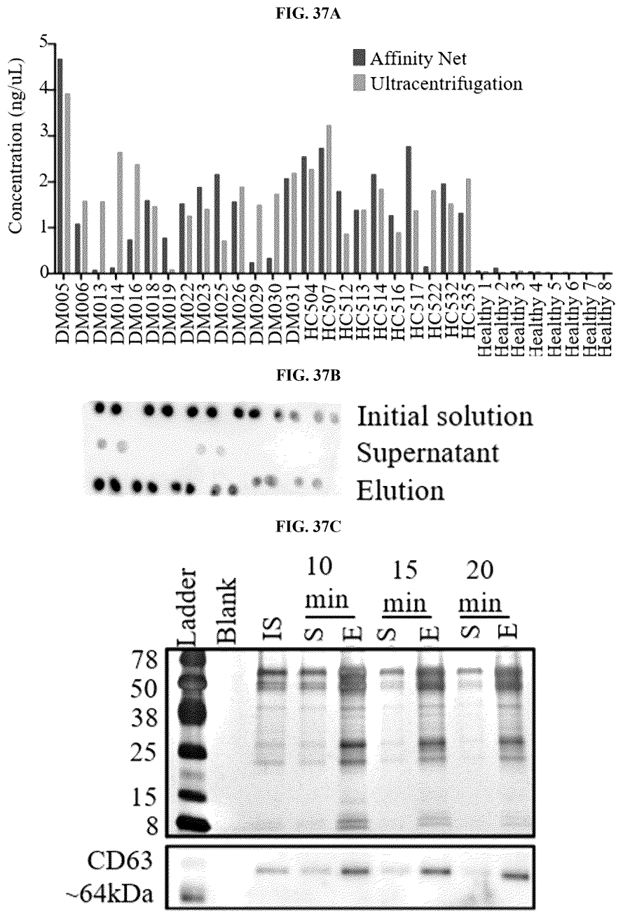

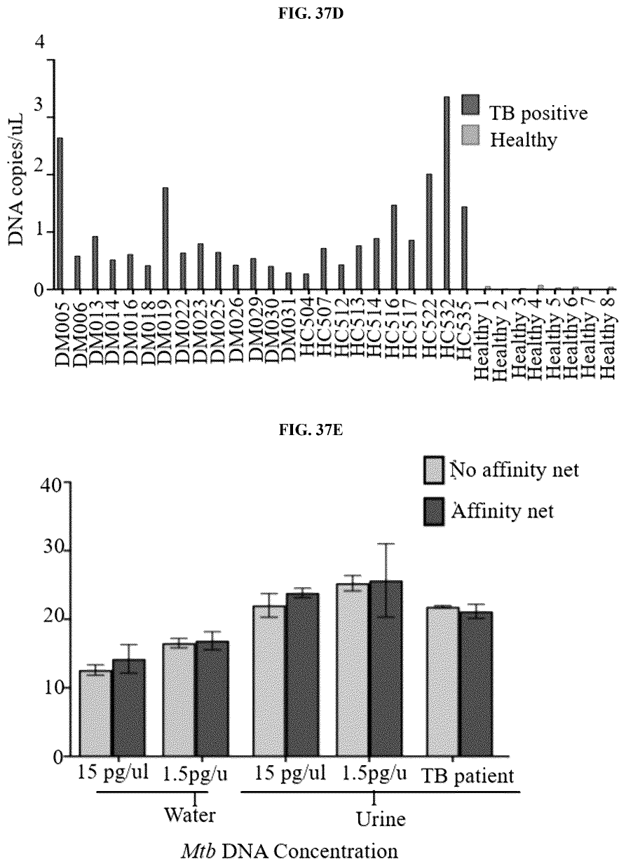

To further validate the affinity net's performance, urine was spiked with known concentrations of purified LAM (50 ng/μL to 0.08 ng/μL, FIG. 37B). LAM is believed to be associated with hydrophobic patches of the Mtb cell wall. The affinity net captured LAM across this concentration range and effectively released it upon elution, demonstrating concentration-dependent recovery (FIG. 37B). Time-course experiments showed that the affinity net captured EVs within 10 minutes of incubation, as demonstrated by SDS PAGE silver staining analysis and detection of CD63, a canonical EV marker, via western blot (FIG. 37C).

These data support the rapid and efficient capture of uEVs and associated biomarkers by the affinity net.

### Example 7: UEVs, Isolated Both Via Ultracentrifugation and the Affinity Net, Contain Mtb DNA

Urinary EVs were evaluated as carriers of Mtb DNA, in particular the RNA polymerase β-subunit gene (RpoB), a common target in nucleic acid amplification-based TB diagnostics. To assess capture of DNA-containing uEVs, urine was analyzed from 24 TB-positive patients and 8 healthy controls. RpoB was detected in uEVs isolated by ultracentrifugation from TB-positive samples and was absent in controls (FIG. 37D).

To quantify DNA capture efficiency, spike-in recovery experiments were performed using synthetic Mtb Strain H37Rv genomic DNA (ATCC #25618D-2™; Lot:70045204) in both water and urine matrices. The poly (NIPAm-coAA) hydrogel (NA=5) coated affinity net recovered >90% of the spiked DNA across physiologically relevant concentrations (15 pg/μL and 1.5 pg/μL), as measured by qPCR (FIG. 37E). The calculated recovery efficiency, based on ΔCT between spiked and captured samples, was 88.6% and 97.8% for 15 pg/μL and 1.5 pg/μL, respectively, in water. In spiked healthy urine, the affinity net achieved recovery rates of 92.3% and 98.4% for the same concentrations. The affinity net enabled RpoB detection in an example TB patient urine (DM007). These results confirm that the affinity net enables efficient DNA capture and detection in both control and TB-positive samples.

Taken together, these data support the utility of the affinity net as a rapid, non-invasive platform for integrated uEV and nucleic acid biomarker capture, enabling downstream TB diagnosis using amplification-based methods.

### Example 8: Discussion

### A Collapsible Urine Collection Device for Conceptually Novel Sample Collection and Shipment Pipeline: Advantages Over Existing Cups

From a clinical laboratory perspective, a collapsible urine collection device has distinct advantages over traditional collection cup containers. The collapsible cup can be folded flat, requiring less space during transport and storage and lowering costs. The collapsible device is not prone to breakage, spillage, and contamination because it eliminates the need to transport and store the liquid part of urine. From the patient perspective, the collapsible cup is easy to use and ship.

Mtb LAM and DNA are Differentially Localized within Urinary EVs

It is hypothesized herein that Mtb LAM is present on the surface of urinary extracellular vesicles (uEVs), whereas Mtb DNA, including the RpoB gene, is encapsulated within the lumen of these vesicles. This conclusion is supported by the results shown in FIGS. 37A-37E, where LAM was detected directly on intact uEVs spotted onto nitrocellulose membranes, whereas RpoB gene amplification by PCR required chemical lysis of the vesicles. LAM was previously measured in vesicles derived from both Mtb and eukaryotic Mtb-infected cells. Its presence in EVs has been visualized using transmission electron microscopy with immunogold-labeled antibodies. LAM integrates into the EV membrane via a phosphatidylinositol (PI) anchor, contributing to the structural integrity of the vesicle and playing roles in host-pathogen interactions, including host cell entry, inhibition of phagosome-lysosome fusion, and intracellular survival. The RpoB gene encodes the β-subunit of RNA polymerase, which is an essential enzyme for bacterial transcription. Given that extracellular vesicles are established carriers of nucleic acids, including genomic DNA and mRNA, the detection of the RpoB gene within the intraluminal contents of urinary EVs aligns with the known EV molecular payload.

uEVs can be Used as an Alternative Sample for Microbiological Testing

The detection of Mtb DNA, including the RpoB gene, in uEVs has significant implications. First, it supports the potential use of uEVs as a noninvasive biomarker source for TB diagnosis. Second, Mtb DNA-containing EVs interact with host immune cells, contributing to immune modulation and pathogenesis. These EVs may also mediate the intercellular transfer of bacterial genetic material, further influencing host-pathogen dynamics. Finally, EV-associated DNA may contain drug resistance genes, although further research is needed to determine the functional relevance and prevalence of these elements in clinical samples. The results reported here support that the uEVs represent a promising non-invasive sample type for microbiological testing that can be coupled to nucleic acid amplification-based assays and immunoassays such as Mtb LAM.

### Macrophage-Derived EVs as a Source of Mtb Components

It is hypothesized herein that these uEVs originate, at least in part, from macrophages infected with Mtb. Infected macrophages release mycobacterial components through EVs, contributing to immune modulation and disease pathogenesis. Mtb lipoglycans, such as LAM, have been detected in EVs released by infected macrophages, where they influence host immune responses. After infection, mycobacterial glycolipids and lipoproteins are trafficked from phagosomes to endocytic compartments and subsequently packaged into EVs. They can modulate host immune responses. Research has shown that post-infection, mycobacterial cell wall traffic out of glycolipids and lipoproteins out of phagosomes. They are disseminated in the endocytic compartments of infected macrophages.

The Affinity Net Matches Ultracentrifugation Performance without Requiring Laboratory Instrumentation

A key advantage of the affinity net platform is its ability to capture uEVs with high efficiency without requiring specialized laboratory equipment. Ultracentrifugation, the current gold standard for EV isolation, requires expensive high-speed centrifuges, trained personnel, and extended processing times. These factors limit its use in standard clinical laboratories and decentralized or resource-constrained settings. In contrast, the affinity net enables rapid and passive capture of uEVs using a functionalized nylon matrix embedded in a simple collection device. EVs captured by the affinity net can be analyzed using molecular and immunoassays. Side-by-side comparisons demonstrated that affinity net performs equivalently to ultracentrifugation in recovering both LAM and RpoB DNA biomarkers from urine. Capture yields exceeded 90% across multiple TB-positive samples and were consistent for both the antigen and gene targets.

This approach eliminates the need for power, refrigeration, and processing infrastructure, supporting its use in field-based and point-of-care diagnostic workflows. These findings support the affinity net as a robust alternative to instrument-intensive methods, advancing the accessibility of molecular diagnostics in low-resource settings.

These findings support user acceptance of a new collapsible urine collection cup for TB detection. uEVs, analyzed with immunoassay or PCR amplification, are a promising fraction for TB diagnosis.

### ENUMERATED EMBODIMENTS

The following exemplary embodiments are provided, the numbering of which is not to be construed as designating levels of importance:

Embodiment 1 provides an affinity matrix comprising:
a biocompatible fibrous material;
a hydrogel; and
an affinity label,
wherein the hydrogel is disposed on the biocompatible fibrous material, and
wherein the affinity label is associated with at least one of the biocompatible fibrous material and the hydrogel.

Embodiment 2 provides the affinity matrix of Embodiment 1, wherein the biocompatible fibrous material comprises nylon.

Embodiment 3 provides the affinity matrix of Embodiment 2, wherein the nylon is a nylon sheet.

Embodiment 4 provides the affinity matrix of Embodiment 2 or 3, wherein the nylon is selected from the group consisting of nylon 6; nylon 6,6; nylon 4,6; nylon 6,9; nylon 6,10; nylon 6,12; nylon 11; and nylon 12.

Embodiment 5 provides the affinity matrix of any one of Embodiments 1-4, wherein the affinity label comprises a dye.

Embodiment 6 provides the affinity matrix of Embodiment 5, wherein the dye is selected from the group consisting of an acid dye, a basic dye, a reactive dye, a disperse dye, a direct dye, a mordant dye, a vat dye, a sulfur dye, an azo dye, an anthraquinone dye, a triphenylmethane dye, a phthalocyanine dye, a polymethine dye, an indigoid dye, a nitro dye, a fluorescent dye, and a natural dye.

Embodiment 7 provides the affinity matrix of Embodiment 5, wherein the dye is selected from the group consisting of Acid Black 48, Acid Red 87, Acid Red 92, Acid Orange 50, Acid Fuchsin, Bismarck Brown, Cibacron Red, Cibacron Yellow, Crystal Violet, Safranin O, Methylene Blue, Pinacyanol Chloride, Fast Blue B+Naphthionic acid, Fast Blue B+Laurent Acid, Fast Blue B+Cleve Acid, Fast Blue B+Peri Acid, Alcian Blue Pyridine variant, Ni Phthalocyanine, Fe Phthalocyanine, Pinacyanol Chloride, Reactive Red 120, Reactive Blue 21, Remazol B Blue, Sudan I, Sudan IV, Sudan Black B, Oil Red O, Acid Black 48, Bismarck Brown Y, Alizarin Cyanin, and Eosin B.

Embodiment 8 provides the affinity matrix of any one of Embodiments 1-7, wherein the hydrogel comprises a crosslinked polymer.

Embodiment 9 provides the affinity matrix of Embodiment 8, wherein the crosslinked polymer is a product of a polymerization reaction between:
at least one vinyl monomer; and
at least one divinyl crosslinker.

Embodiment 10 provides the affinity matrix of Embodiment 9, wherein each vinyl monomer is independently a compound of formula (I):

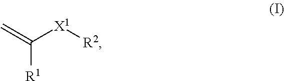

wherein:
   R¹ is selected from the group consisting of H, optionally substituted C₁-C₃ alkyl, and a halogen;
   R² is selected from the group consisting of N(R^{A})(R^{B}), OR^{A}, CN, NO₂, halogen, optionally substituted C₆-C₁₀ aryl, and optionally substituted C₂-C₁₀ heteroaryl;
   X¹ is selected from the group consisting of a bond, —C(R^{3a})(R^{3b})—, —C(═O)—, —P(═O)OR^{A}—, —S(═O)—, and —S(═O)₂—;
   R^{3a} and R^{3b} are each independently selected from the group consisting of H, optionally substituted C₁-C₃ alkyl, and a halogen; and
   each occurrence of R^{A} and R^{B} are each independently selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl, or R^{A} and R^{B} can combine with the nitrogen atom to which they are bound to form an optionally substituted C₂-C₈ heterocycloalkyl.

Embodiment 11 provides the affinity matrix of Embodiment 9 or 10, wherein each vinyl monomer is independently selected from the group consisting of N-isopropylacrylamide, acrylamide, methacrylamide, vinyl sulfonic acid, allylamine, N-vinylpyrrolidone, acrylic acid, methacrylic acid, hydroxyethyl acrylate, hydroxyethyl methacrylate, vinyl acetate, styrene, 2-acrylamido-2-methyl-1-propanesulfonic acid, acrylonitrile, methacrylonitrile, vinyl imidazole, vinyl phosphonic acid, vinyl chloride, vinylidene chloride, glycidyl methacrylate, dimethylaminoethyl acrylate, dimethylaminoethyl methacrylate, and itaconic acid.

Embodiment 12 provides the affinity matrix of any one of Embodiments 9-11, wherein the vinyl monomer comprises at least one of:
(a) N-isopropylacrylamide;
(b) N-isopropylacrylamide and allylamine;
(c) N-isopropylacrylamide and vinyl sulfonic acid; and
(d) acrylamide.

Embodiment 13 provides the affinity matrix of any one of Embodiments 9-12, wherein each divinyl monomer is independently a compound of formula (II):

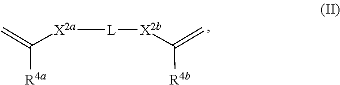

wherein:
   R^{4a} and R^{4b} are each independently selected from the group consisting of H, optionally substituted C₁-C₃ alkyl, and a halogen;
   X^{2a} and X^{2b} are each independently selected from the group consisting of a bond, —C(R^{5a})(R^{5b})—, —C(═O)—, —P(═O)OR^{C}—, —S(═O)—, and —S(═O)₂—;
   L is at least one selected from the group consisting of —N(R^{C})—, —O—, —C(═O)—, -(optionally substituted C₁-C₆ alkylenyl)-, -(optionally substituted C₁-C₆ heteroalkylenyl)-, -(optionally substituted C₃-C₈ cycloalkylenyl)-, -(optionally substituted C₂-C₈ heterocycloalkylenyl)-, -(optionally substituted C₆-C₁₀ arylenyl)-, and -(optionally substituted C₂-C₈ heteroarylenyl)-;
   R^{5a} and R^{5b} are each independently selected from the group consisting of H, optionally substituted C₁-C₃ alkyl, and a halogen; and
   each occurrence of R^{C} is independently selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl.

Embodiment 14 provides the affinity matrix of any one of Embodiments 9-13, wherein each divinyl crosslinker is independently selected from the group consisting of N,N′-methylenebis(acrylamide), N,N′-ethylenebis(acrylamide), ethylene glycol diacrylate, ethylene glycol dimethacrylate, diethylene glycol diacrylate, diethylene glycol dimethacrylate, polyethylene glycol diacrylate, polyethylene glycol dimethacrylate, glycerol dimethacrylate, neopentyl glycol diacrylate, neopentyl glycol dimethacrylate, divinylbenzene, diallyl phthalate, and diallyl carbonate.

Embodiment 15 provides the affinity matrix of any one of Embodiments 5-14, affinity matrix of Embodiment 5, wherein the affinity matrix comprises about 0.005 to about 0.050 mmol dye per milligram fibrous material, optionally wherein the affinity matrix comprises about 0.0164 mmol dye per milligram fibrous material.

Embodiment 16 provides the affinity matrix of any one of Embodiments 1-15, wherein the vinyl monomer and divinyl crosslinker have a molar ratio of about 100:1 to about 100:10, optionally wherein the vinyl monomer and divinyl crosslinker have a molar ratio of about 100:3.

Embodiment 17 provides the affinity matrix of any one of Embodiments 1-16, wherein the association of the dye with the fibrous material or hydrogel comprises a non-covalent bonding interaction (e.g., hydrogen bonding interaction, Van der Waals interaction, and/or ionic bonding interaction).

Embodiment 18 provides the affinity matrix of any one of Embodiments 1-17, wherein the association of the dye with the fibrous material or hydrogel comprises a covalent bond between the dye and the fibrous material or hydrogel.

Embodiment 19 provides a method for preparing the affinity matrix of any one of Embodiments 1-18, the method comprising:
contacting a fibrous material sheet with an affinity label to provide an affinity labeled fibrous material;
placing the affinity labeled fibrous material in a sealable vessel; and
contacting the affinity labeled fibrous material with a monomer solution comprising a vinyl monomer, a divinyl crosslinker, and a radical initiator under an anaerobic condition.

Embodiment 20 provides the method of Embodiment 19, wherein the contacting occurs in the presence of a bidentate ligand, optionally wherein the bidentate ligand comprises tetramethylenediamine (TEMED).

Embodiment 21 provides a method for separating an analyte from a sample, the method comprising:
contacting a liquid sample with the affinity matrix of any one of Embodiment 1-18 to provide an analyte-adsorbed affinity matrix and a supernatant;
separating the analyte-adsorbed affinity matrix and the supernatant; and
contacting the analyte-adsorbed affinity matrix with an eluting solution to provide an analyte solution.

Embodiment 22 provides the method of any one of Embodiments 19-21, wherein the contacting occurs for a period of about 1 to about 60 minutes, optionally wherein the contacting occurs for about 10, about 15, or about 30 minutes.

Embodiment 23 provides the method of Embodiment 21 or 22, wherein the separating comprises decanting, pipetting off, or filtering the supernatant from the analyte-adsorbed affinity matrix.

Embodiment 24 provides the method of any one of Embodiments 21-23, wherein the eluting solution comprises a buffer solution, optionally wherein the buffer solution comprises urea and sodium dodecyl sulfate (SDS) (e.g., 2M urea and 3.85% SDS).

Embodiment 25 provides the method of any one of Embodiments 21-24, wherein the analyte solution is subjected to an analytical assay.

Embodiment 26 provides the method of Embodiment 25, wherein the analytical assay is selected from the group consisting of mass spectrometry, spectrophotometry, chromatography, an immunoassay, and a nucleic acid amplification assay.

Embodiment 27 provides the method of any one of Embodiments 21-26, wherein the liquid sample and affinity matrix have a ratio of about 500 μL liquid sample/cm² affinity matrix to about 50 μL liquid sample/cm² affinity matrix.

Embodiment 28 provides the method of any one of Embodiments 21-27, wherein the liquid sample comprises a liquid biological sample or a liquid environmental sample.

Embodiment 29 provides the method of Embodiment 28, wherein the liquid biological sample comprises urine, blood, plasma, serum, saliva, cerebrospinal fluid, vaginal fluid, semen, or a cell culture medium.

Embodiment 30 provides the method of Embodiment 28, wherein the liquid environmental sample comprises wastewater, surface water, groundwater, river water, lake water, ocean water, or industrial effluent.

Embodiment 31 provides the method of any one of Embodiments 21-30, wherein the analyte is selected from the group consisting of a protein, peptide, nucleic acid, carbohydrate, lipid, lipid particle, and small molecule.

Embodiment 32 provides the method of Embodiment 31, wherein the lipid particle comprises a urinary extracellular vesicle (uEV).

Embodiment 33 provides the method of any one of Embodiments 21-32, wherein the analyte is a biomarker of a pathogen or a subject infected with the pathogen, optionally wherein the pathogen is selected from the group consisting of a bacterium, virus, fungus, and protozoan.

Embodiment 34 provides the method of Embodiment 33, wherein the biomarker comprises lipoarabinomannan (LAM), Mycobacterial (Mtb) DNA/RNA fragments, Mtb proteins, Borrelia DNA/RNA fragments, Borrelia outer surface proteins (OspA, OspC, OspE, RimM, and FliM), PGM (gpmA), MutL, HDDP, OEPF, or L20.

Embodiment 35 provides the method of Embodiment 33 or 34, wherein the biomarker comprises a urinary cytokine or chemokine, optionally wherein the biomarker is selected from the group consisting of CD9, CD81, CD59, CD44, CD59, CD63, tetraspanin-1, MIF, LAMP2, IGLL5, MAN1A1, HLA-Cw1α, HLA-DRB1-4 3 (isoform X2), and CD14.

Embodiment 36 provides the method of any one of Embodiments 33-35, wherein the pathogen is Borrelia burgdorferi or Mycobacterium tuberculosis.

Embodiment 37 provides a method for diagnosing a disease or disorder in a subject, the method comprising:
contacting a liquid biological sample obtained from the subject with the affinity matrix of any one of Embodiments 1-18 to provide an analyte-adsorbed affinity matrix and a supernatant;
separating the analyte-adsorbed affinity matrix and the supernatant;
contacting the analyte-adsorbed affinity matrix with an eluting solution to provide an analyte solution; and
detecting a biomarker associated with the disease or disorder in the analyte solution.

Embodiment 38 provides a method for treating a disease or disorder in a subject, the method comprising:
contacting a liquid biological sample obtained from the subject with the affinity matrix of any one of Embodiments 1-18 to provide an analyte-adsorbed affinity matrix and a supernatant;
separating the analyte-adsorbed affinity matrix and the supernatant;
contacting the analyte-adsorbed affinity matrix with an eluting solution to provide an analyte solution;
detecting a biomarker associated with the disease or disorder in the analyte solution; and
administering to the subject a therapeutic agent effective to treat the disease or disorder.

Embodiment 39 provides the method of Embodiment 38, wherein the disease or disorder comprises an infection with a pathogen, optionally wherein the pathogen is selected from the group consisting of a bacterium, virus, fungus, and protozoan.

Embodiment 40 provides the method of Embodiment 39, wherein the analyte solution comprises a biomarker of the pathogen or the subject.

Embodiment 41 provides the method of Embodiment 39 or 40, wherein the infection comprises a bacterial infection.

Embodiment 42 provides the method of Embodiment 41, wherein the bacterial infection comprises borreliosis (e.g., Lyme disease and neuroborreliosis) or tuberculosis.

Embodiment 43 provides a kit comprising:
the affinity matrix of any one of Embodiments 1-18;
a liquid collection device, optionally the device is collapsible; and
instructions for preparing and storing an analyte-adsorbed affinity matrix in the liquid collection device.

Embodiment 44 provides the kit of Embodiment 43, wherein the liquid collection device comprises a cup, optionally wherein the liquid collection device comprise a polypropylene sheet.

Embodiment 45 provides the kit of Embodiment 43 or 44, wherein the kit further comprises an envelope configured to store the liquid collection device comprising the analyte-adsorbed affinity matrix.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

What is claimed is:

1. An affinity matrix comprising:
a biocompatible fibrous material;
a hydrogel; and
an affinity label,
wherein the hydrogel is disposed on the biocompatible fibrous material, and
wherein the affinity label is associated with at least one of the biocompatible fibrous material and the hydrogel.

2. The affinity matrix of claim 1, wherein the biocompatible fibrous material comprises nylon.

3. The affinity matrix of claim 1, wherein the affinity label comprises a dye.

4. The affinity matrix of claim 1, wherein the hydrogel comprises a crosslinked polymer.

5. The affinity matrix of claim 4, wherein the crosslinked polymer is a product of a polymerization reaction between:
at least one vinyl monomer; and
at least one divinyl crosslinker.

6. The affinity matrix of claim 5, wherein each vinyl monomer is independently a compound of formula (I): 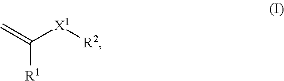
wherein:
R¹ is selected from the group consisting of H, optionally substituted C₁-C₃ alkyl, and a halogen;
R² is selected from the group consisting of N(R^{A})(R^{B}), OR^{A}, CN, NO₂, halogen, optionally substituted C₆-C₁₀ aryl, and optionally substituted C₂-C₁₀ heteroaryl;
X¹ is selected from the group consisting of a bond, —C(R^{3a})(R^{3b})—, —C(═O)—, —P(═O)OR^{A}—, —S(═O)—, and —S(═O)₂—;
R^{3a} and R^{3b} are each independently selected from the group consisting of H, optionally substituted C₁-C₃ alkyl, and a halogen; and
each occurrence of R^{A} and R^{B} are each independently selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl, or R^{A} and R^{B} can combine with the nitrogen atom to which they are bound to form an optionally substituted C₂-C₈ heterocycloalkyl.

7. The affinity matrix of claim 5, wherein each divinyl monomer is independently a compound of formula (II): 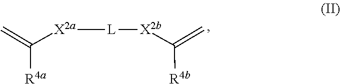
wherein:
R^{4a} and R^{4b} are each independently selected from the group consisting of H, optionally substituted C₁-C₃ alkyl, and a halogen;
X^{2a} and X^{2b} are each independently selected from the group consisting of a bond, —C(R^{5a})(R^{5b})—, —C(═O)—, —P(═O)OR^{C}—, —S(═O)—, and —S(═O)₂—;
L is at least one selected from the group consisting of —N(R^{C})—, —O—, —C(═O)—, -(optionally substituted C₁-C₆ alkylenyl)-, -(optionally substituted C₁-C₆ heteroalkylenyl)-, -(optionally substituted C₃-C₈ cycloalkylenyl)-, -(optionally substituted C₂-C₈ heterocycloalkylenyl)-, -(optionally substituted C₆-C₁₀ arylenyl)-, and -(optionally substituted C₂-C₈ heteroarylenyl)-;
R^{5a} and R^{5b} are each independently selected from the group consisting of H, optionally substituted C₁-C₃ alkyl, and a halogen; and
each occurrence of R^{C} is independently selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl.

8. The affinity matrix of claim 3, wherein the affinity matrix comprises about 0.005 to about 0.050 mmol dye per milligram fibrous material, optionally wherein the affinity matrix comprises about 0.0164 mmol dye per milligram fibrous material.

9. The affinity matrix of claim 1, wherein the vinyl monomer and divinyl crosslinker have a molar ratio of about 100:1 to about 100:10, optionally wherein the vinyl monomer and divinyl crosslinker have a molar ratio of about 100:3.

10. A method for preparing the affinity matrix of claim 1, the method comprising:
contacting a fibrous material sheet with an affinity label to provide an affinity labeled fibrous material;
placing the affinity labeled fibrous material in a sealable vessel; and
contacting the affinity labeled fibrous material with a monomer solution comprising a vinyl monomer, a divinyl crosslinker, and a radical initiator under an anaerobic condition.

11. A method for separating an analyte from a sample, the method comprising:
contacting a liquid sample with the affinity matrix of claim 1 to provide an analyte-adsorbed affinity matrix and a supernatant;
separating the analyte-adsorbed affinity matrix and the supernatant; and
contacting the analyte-adsorbed affinity matrix with an eluting solution to provide an analyte solution.

12. The method of claim 11, wherein the liquid sample comprises a liquid biological sample or a liquid environmental sample.

13. The method of claim 12, wherein the liquid biological sample comprises urine, blood, plasma, serum, saliva, cerebrospinal fluid, vaginal fluid, semen, or a cell culture medium.

14. The method of claim 11, wherein the analyte is a biomarker of a pathogen or a subject infected with the pathogen.

15. The method of claim 14, wherein the biomarker comprises lipoarabinomannan (LAM), Mycobacterial (Mtb) DNA/RNA fragments, Mtb proteins, Borrelia DNA/RNA fragments, Borrelia outer surface proteins (OspA, OspC, OspE, RimM, and FliM), PGM (gpmA), MutL, HDDP, OEPF, or L20.

16. The method of claim 14, wherein the biomarker comprises a urinary cytokine or chemokine, optionally wherein the biomarker is selected from the group consisting of CD9, CD81, CD59, CD44, CD59, CD63, tetraspanin-1, MIF, LAMP2, IGLL5, MAN1A1, HLA-Cw1α, HLA-DRB1-4 3 (isoform X2), and CD14.

17. The method of claim 14, wherein the pathogen is Borrelia burgdorferi or Mycobacterium tuberculosis.

18. A method for diagnosing a disease or disorder in a subject, the method comprising:
contacting a liquid biological sample obtained from the subject with the affinity matrix of claim 1 to provide an analyte-adsorbed affinity matrix and a supernatant;
separating the analyte-adsorbed affinity matrix and the supernatant;
contacting the analyte-adsorbed affinity matrix with an eluting solution to provide an analyte solution; and
detecting a biomarker associated with the disease or disorder in the analyte solution.

19. A method for treating a disease or disorder in a subject, the method comprising:
contacting a liquid biological sample obtained from the subject with the affinity matrix of claim 1 to provide an analyte-adsorbed affinity matrix and a supernatant;
separating the analyte-adsorbed affinity matrix and the supernatant;
contacting the analyte-adsorbed affinity matrix with an eluting solution to provide an analyte solution;
detecting a biomarker associated with the disease or disorder in the analyte solution; and
administering to the subject a therapeutic agent effective to treat the disease or disorder.

20. A kit comprising:
the affinity matrix of claim 1;
a liquid collection device, optionally the device is collapsible; and
instructions for preparing and storing an analyte-adsorbed affinity matrix in the liquid collection device.


SEQUENCE LISTING (8 sequences)

SEQ ID NO: 1 - protein, 27 aa, synthetic construct
YFDNLSEEDVLKLNIPTGIPLVYELDK

SEQ ID NO: 2 - protein, 19 aa, synthetic construct
LGEVVAFFEYLNSVFLEVR

SEQ ID NO: 3 - protein, 16 aa, synthetic construct
LENTAMPLVAEIGEVK

SEQ ID NO: 4 - protein, 22 aa, synthetic construct
INTYDIPQNNNLETEDVNEPNK

SEQ ID NO: 5 - protein, 19 aa, synthetic construct
IVDIDESNPNLPYINYIIK

SEQ ID NO: 6 - protein, 13 aa, synthetic construct
NYLNEPSRDIITK

SEQ ID NO: 7 - protein, 11 aa, synthetic construct
SLEVLGNEYTK

SEQ ID NO: 8 - protein, 21 aa, synthetic construct
ISAALSDTGVTYSRFIEGLLK